SEQUENCE LISTING (102 sequences)

SEQ ID NO: 1 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 2 - DNA, 2448 bp, Homo sapiens
agaacacttacaggatgtgtgtagtgtggcatgacagagaactttggtttcctttaatgtgactgtagacctggcagtgttactataagaatcactggcaatcagacacccgggtgtgctgagctagcactcagtgggggcggctactgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgttaaaaaaaaaaaaaaaaaa

SEQ ID NO: 3 - protein, 142 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGEWTFACHLYE

SEQ ID NO: 4 - DNA, 1012 bp, Homo sapiens
actgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccaggtgagtggacatttgcatgccatctttatgaataagatttatctgtggatcatattaaaggtactgattgttctcatctctgacttccctaattatagccctggaggagggccactaagacctaaagtttaacaggccccattggtgatgctcagtgatatttaacaccttctctctgttttaaaactcatgggtgtgcctgggcgtggtggctcgcgcctctggtcccagcactttgggaggctgaggccggtggatcatgaggtcaggaattcgagaccagcctggccaacatggtaaaaccttgtctccactaaaaatacaaaaaattagccaggcatggttacgggagcctgtaattctagctacttggggggctgaagcaggagaatcacttgaacctggaagtcggaggttgcggtaagccaagatctcgccattgtactccagcctggctgacaagagtgaaactctgtcccaaaaaaaaaaaaaaaaaaaaaaaaaaaaaa

SEQ ID NO: 5 - protein, 598 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 6 - DNA, 2940 bp, Homo sapiens
atttggttgaaagaaaacccacaatccagtgtcaagaaagaagtcaacttttcttcccctacttccctgcatttctcctctgtgctcactgccacacacagctcaacctggacagcacagccagaggcgagatgcttctctgctgatctgagtctgcctgcagcatggacctgggtcttccctgaagcatctccagggctggagggacgactgccatgcaccgagggctcatccatccgcagagcagggcagtgggaggagacgccatgacccccatcgtcacagtcctgatctgtctcgggctgagtctgggccccaggacccgcgtgcagacagggaccatccccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagatgaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcatggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctgcaggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactagcccggagggtacgcagactccacactcagtagaaggagactcaggactgctgaaggcacgggagctgcccccagtggacaccaatgaaccccagtcagcctggacccctaacaaagaccatgaggagatgctgggaactttgggactcacttgattctgcagtcgaaataactaatatccctacattttttaattaaagcaacagacttctcaataatcaatgagttaaccgagaaaactaaaatcagaagtaagaatgtgctttaaactgaatcacaatataaatattacacatcacacaatgaaattgaaaaagtacaaaccacaaatgaaaaaagtagaaacgaaaaaaaaaaactaggaaatgaatgacgttggctttcgtataaggaatttagaaaaagaataaccaattattccaaatgaaggtgtaagaaagggaataagaagaagaagagttgctcatgaggaaaaaccaaaacttgaaaattcaacaaagccaatgaagctcattcttgaaaatattaattacagtcataaatcctaactacattgagcaagagaaagaaagagcaggcacgcatttccatatgggagtgagccagcagacagcccagcagatcctacacacattttcacaaactaaccccagaacaggctgcaaacctataccaatatactagaaaatgcagattaaatggatgaaatattcaaaactggagtttacataatgaacgtaagagtaatcagagaatctgactcattttaaatgtgtgtgtatgtgtgtgtatatatatgtgtgtgtgtgtgtgtgtgtgtgtgtgtgaaaaacattgactgtaataaaaatgttcccatcgtaaaaaaaaaaaaaaaa

SEQ ID NO: 7 - protein, 597 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 8 - DNA, 2937 bp, Homo sapiens
atttggttgaaagaaaacccacaatccagtgtcaagaaagaagtcaacttttcttcccctacttccctgcatttctcctctgtgctcactgccacacacagctcaacctggacagcacagccagaggcgagatgcttctctgctgatctgagtctgcctgcagcatggacctgggtcttccctgaagcatctccagggctggagggacgactgccatgcaccgagggctcatccatccgcagagcagggcagtgggaggagacgccatgacccccatcgtcacagtcctgatctgtctcgggctgagtctgggccccaggacccgcgtgcagacagggaccatccccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagatgaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcatggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactagcccggagggtacgcagactccacactcagtagaaggagactcaggactgctgaaggcacgggagctgcccccagtggacaccaatgaaccccagtcagcctggacccctaacaaagaccatgaggagatgctgggaactttgggactcacttgattctgcagtcgaaataactaatatccctacattttttaattaaagcaacagacttctcaataatcaatgagttaaccgagaaaactaaaatcagaagtaagaatgtgctttaaactgaatcacaatataaatattacacatcacacaatgaaattgaaaaagtacaaaccacaaatgaaaaaagtagaaacgaaaaaaaaaaactaggaaatgaatgacgttggctttcgtataaggaatttagaaaaagaataaccaattattccaaatgaaggtgtaagaaagggaataagaagaagaagagttgctcatgaggaaaaaccaaaacttgaaaattcaacaaagccaatgaagctcattcttgaaaatattaattacagtcataaatcctaactacattgagcaagagaaagaaagagcaggcacgcatttccatatgggagtgagccagcagacagcccagcagatcctacacacattttcacaaactaaccccagaacaggctgcaaacctataccaatatactagaaaatgcagattaaatggatgaaatattcaaaactggagtttacataatgaacgtaagagtaatcagagaatctgactcattttaaatgtgtgtgtatgtgtgtgtatatatatgtgtgtgtgtgtgtgtgtgtgtgtgtgtgaaaaacattgactgtaataaaaatgttcccatcgtaaaaaaaaaaaaaaaa

SEQ ID NO: 9 - protein, 281 aa, Mus musculus
MFSGLTLNCVLLLLQLLLARSLENAYVFEVGKNAYLPCSYTLSTPGALVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIHIGVGVSAGLTLALIIGVLILKWYSCKKKKLSSLSLITLANLPPGGLANAGAVRIRSEENIYTIEENVYEVENSNEYYCYVNSQQPS

SEQ ID NO: 10 - DNA, 2725 bp, Mus musculus
  allele (0)...(0): Synthetic construct
accattttaaccgaggagctaaagctatccctacacagagctgtccttggatttcccctgccaagtactcatgttttcaggtcttaccctcaactgtgtcctgctgctgctgcaactactacttgcaaggtcattggaaaatgcttatgtgtttgaggttggtaagaatgcctatctgccctgcagttacactctatctacacctggggcacttgtgcctatgtgctggggcaagggattctgtccttggtcacagtgtaccaacgagttgctcagaactgatgaaagaaatgtgacatatcagaaatccagcagataccagctaaagggcgatctcaacaaaggagacgtgtctctgatcataaagaatgtgactctggatgaccatgggacctactgctgcaggatacagttccctggtcttatgaatgataaaaaattagaactgaaattagacatcaaagcagccaaggtcactccagctcagactgcccatggggactctactacagcttctccaagaaccctaaccacggagagaaatggttcagagacacagacactggtgaccctccataataacaatggaacaaaaatttccacatgggctgatgaaattaaggactctggagaaacgatcagaactgctatccacattggagtgggagtctctgctgggttgaccctggcacttatcattggtgtcttaatccttaaatggtattcctgtaagaaaaagaagttatcgagtttgagccttattacactggccaacttgcctccaggagggttggcaaatgcaggagcagtcaggattcgctctgaggaaaatatctacaccatcgaggagaacgtatatgaagtggagaattcaaatgagtactactgctacgtcaacagccagcagccatcctgaccgcctctggactgccacttttaaaggctcgccttcatttctgactttggtatttccctttttgaaaactatgtgatatgtcacttggcaacctcattggaggttctgaccacagccactgagaaaagagttccagttttctggggataattaactcacaaggggattcgactgtaactcatgctacattgaaatgctccattttatccctgagtttcagggatcggatctcccactccagagacttcaatcatgcgtgttgaagctcactcgtgctttcatacattaggaatggttagtgtgatgtctttgagacatagaggtttgtggtatatctgcaaagctcctgaacaggtagggggaataaagggctaagataggaaggtgaggttctttgttgatgttgaaaatctaaagaagttggtagcttttctagagatttctgaccttgaaagattaagaaaaagccaggtggcatatgcttaacactatataacttgggaaccttaggcaggagggtgataagttcaaggtcagccagggctatgctggtaagactgtctcaaaatccaaagacgaaaataaacatagagacagcaggaggctggagatgaggctcggacagtgaggtgcattttgtacaagcacgaggaatctatatttgatcgtagaccccacatgaaaaagctaggcctggtagagcatgcttgtagactcaagagatggagaggtaaaggcacaacagatccccggggcttgcgtgcagtcagcttagcctaggtgctgagttccaagtccacaagagtccctgtctcaaagtaagatggactgagtatctggcgaatgtccatgggggttgtcctctgctctcagaagagacatgcacatgaacctgcacacacacacacacacacacacacacacacacacacacacacacacacacacacatgaaatgaaggttctctctgtgcctgctacctctctataacatgtatctctacaggactctcctctgcctctgttaagacatgagtgggagcatggcagagcagtccagtaattaattccagcactcagaaggctggagcagaagcgtggagagttcaggagcactgtgcccaacactgccagactcttcttacagaagaaaaaggttacccgcaagcagcctgctgtctgtaaaaggaaaccctgcgaaaggcaaactttgactgttgtgtgctcaaggggaactgactcagacaacttctccattcctggaggaaactggagctgtttctgacagaagaacaaccggtgactgggacatacgaaggcagagctcttgcagcaatctatatagtcagcaaaatattctttgggaggacagtcgtcaccaaattgatttccaagccggtggacctcagtttcatctggcttacagctgcctgcccagtgcccttgatctgtgctggctcccatctataacagaatcaaattaaatagaccccgagtgaaaatattaagtgagcagaaaggtagctttgttcaaagatttttttgcattggggagcaactgtgtacatcagaggacatctgttagtgaggacaccaaaacctgtggtaccgttttttcatgtatgaattttgttgtttaggttgcttctagctagctgtggaggtcctggctttcttaggtgggtatggaagggagaccatctaacaaaatccattagagataacagctctcatgcagaagggaaaactaatctcaaatgttttaaagtaataaaactgtactggcaaagtactttgagcatatttaaa

SEQ ID NO: 11 - DNA, 631 bp, Mus musculus
  allele (0)...(0): Synthetic construct
aagcagtggtatcaacgcagagtacgcggggcatcatcttctcatagagcctccatcagagcatggctgtcctggcattactcttctgcctggtaacattcccaagctgtatcctttcccaggtgcagctgaaggagtcaggacctggcctggtggcgccctcacagagcctgtccatcacatgcaccgtctcagggttctcattaaccggctatggtgttacctgggttcgccagcctccaggaaagggtctggagtggctgggaatgatatggggtgatggaaacacagactataattcaggtctcaaatccagactgaacatcagcaaggacaactccaagagccaagttttcttaaaaatgaacagtctgcaaactgatgacacagccaggtactactgtgccaggtcttattactacggtccccctgactactggggccaaggcaccactctcacagtctcctcagccaaaacgacacccccatctgtctatccactggcccctggatctgctgcccaaaactaactccatggtgaccctgggatgcctggtcaagggctatttccctgagccagtgacagtgacctggaactctggatccctgtccagcggtgtgcacaccttcc

SEQ ID NO: 12 - protein, 136 aa, Mus musculus
MAVLALLFCLVTFPSCILSQVQLKESGPGLVAPSQSLSITCTVSGFSLTGYGVTWVRQPPGKGLEWLGMIWGDGNTDYNSGLKSRLNISKDNSKSQVFLKMNSLQTDDTARYYCARSYYYGPPDYWGQGTTLTVSS

SEQ ID NO: 13 - DNA, 507 bp, Mus musculus
aagcagtggtatcaacgcagagtacgcggggcatcagacaggctgggcagcaagatggaatcacagacccaggtcctcatgtttcttctgctctgggtatctggtgcctgtgcagacattgtgatgacacagtctccatcctccctggctatgtcagtaggacagaaggtcactatgagctgcaagtccagtcagagccttctaaatagtagaagtcaaaagaactatttggcctggtaccagcggaaaccaggacagtctcctaaacttctgctatactttgcatccactagggaatctggggtccctgatcgcttcataggcagtggatctgggacagatttcactcttaccatcagcagtgtgcaggctgaagacctggcagattacttctgtcaccaacattataacactccgtacacgttcggaggggggaccaagctggaaattaaacgggctgatgctgcaccaactgtatccatcttcccaccatccagtgagcagtta

SEQ ID NO: 14 - protein, 133 aa, Mus musculus
MESQTQVLMFLLLWVSGACADIVMTQSPSSLAMSVGQKVTMSCKSSQSLLNSRSQKNYLAWYQRKPGQSPKLLLYFASTRESGVPDRFIGSGSGTDFTLTISSVQAEDLADYFCHQHYNTPYTFGGGTKLEIK

SEQ ID NO: 15 - protein, 8 aa, Artificial Sequence
  Synthetic construct
GFSLTGYG

SEQ ID NO: 16 - protein, 7 aa, Artificial Sequence
  Synthetic construct
IWGDGNT

SEQ ID NO: 17 - protein, 11 aa, Artificial Sequence
  Synthetic construct
ARSYYYGPPDY

SEQ ID NO: 18 - protein, 12 aa, Artificial Sequence
  Synthetic construct
QSLLNSRSQKNY

SEQ ID NO: 19 - protein, 3 aa, Artificial Sequence
  Synthetic construct
FAS

SEQ ID NO: 20 - protein, 9 aa, Artificial Sequence
  Synthetic construct
HQHYNTPYT

SEQ ID NO: 21 - protein, 118 aa, Mus musculus
QIQLQQSGPELVKPGTSVKISCKASGYTFTDYYINWVKQRPGQGLEWIGWIYPGSGNTKYNEKFKGKATLTVDTSSSTAYMQLSSLTSEDSAVYFFARGGKYYAMDYWGQGTSVTVSS

SEQ ID NO: 22 - protein, 107 aa, Mus musculus
NIVMTQTPKFLLISAGDRVTITCKASQSVGNNVAWYQQKPGQSPKLLIYYASNRYTGVPDRFTGSAYGTDFTFTITTVQAEDLAVYFCQQDYSSPYTFGGGTKLEIK

SEQ ID NO: 23 - protein, 121 aa, Mus musculus
EVQLVESGGDLVKPGGSLKLSCAASGFTFSNYGMSWVRQTPDKRLEWVATISSGGSNTYFPDSVKGRFTISRDNAKNTLYLQMSSLKSEDTAMYYCARHGTSMIKEWFAYWGQGTLVTVSA

SEQ ID NO: 24 - protein, 107 aa, Mus musculus
DIVMTQSPVTLSVTPGDRVSLSCRASQSIGDYLHWYQQKSHESPRLLIKYASQSISGIPSRFSGSGSGSDFTLNINSVEPEDVGVYYCQNSHSFPPTFGGGTRLEIK

SEQ ID NO: 25 - protein, 121 aa, Mus musculus
EVQLVESGGDLVKPGGSLKLSCAASGFTFSNHGMSWVRQTPDKRLEWVATISSGGSNTYFPDSVKGRFTISRDNVKNSLYLQMSSLKSEDTAMYYCARHGTSMIKEWFAYWGQGTLVTVSA

SEQ ID NO: 26 - protein, 107 aa, Mus musculus
DIVMTQSPVTLSVTPGDRVSLSCRASQSIGDYLHWYQQKSHESPRLLIKYASQSISGIPSRFSGSGSGSDFTLNINSVEPEDVGVYYCQHSHSFPPTFGGGTRLEIK

SEQ ID NO: 27 - protein, 10 aa, Artificial Sequence
  Synthetic construct
GYTFTDYYIN

SEQ ID NO: 28 - protein, 17 aa, Artificial Sequence
  Synthetic construct
WIYPGSGNTKYNEKFKG

SEQ ID NO: 29 - protein, 9 aa, Artificial Sequence
  Synthetic construct
GGKYYAMDY

SEQ ID NO: 30 - protein, 11 aa, Artificial Sequence
  Synthetic construct
KASQSVGNNVA

SEQ ID NO: 31 - protein, 7 aa, Artificial Sequence
  Synthetic construct
YASNRYT

SEQ ID NO: 32 - protein, 9 aa, Artificial Sequence
  Synthetic construct
QQDYSSPYT

SEQ ID NO: 33 - protein, 10 aa, Artificial Sequence
  Synthetic construct
GFTFSNYGMS

SEQ ID NO: 34 - protein, 17 aa, Artificial Sequence
  Synthetic construct
TISSGGSNTYFPDSVKG

SEQ ID NO: 35 - protein, 12 aa, Artificial Sequence
  Synthetic construct
HGTSMIKEWFAY

SEQ ID NO: 36 - protein, 11 aa, Artificial Sequence
  Synthetic construct
RASQSIGDYLH

SEQ ID NO: 37 - protein, 7 aa, Artificial Sequence
  Synthetic construct
YASQSIS

SEQ ID NO: 38 - protein, 9 aa, Artificial Sequence
  Synthetic construct
QNSHSFPPT

SEQ ID NO: 39 - protein, 10 aa, Artificial Sequence
  Synthetic construct
GFTFSNHGMS

SEQ ID NO: 40 - protein, 9 aa, Artificial Sequence
  Synthetic construct
QHSHSFPPT

SEQ ID NO: 41 - protein, 121 aa, Mus musculus
QIQLVQSGPELKKPGETVKISCKASGYTFTTYGMSWVKQAPGKGLKLMGWINTYSGAPTYADDFKGRFAFSLETSASAAYLQINNLKNEDTATYFCARKPPHYYVNSFDYWGQGTTLTVSS

SEQ ID NO: 42 - protein, 121 aa, Artificial Sequence
  Synthetic construct
QVQLVQSGSELKKPGAPVKVSCKASGYTFTTYGMSWVRQAPGQGLEWMGWINTYSGAPTYADDFKGRFVFSLDTSVSTAYLQISSLKAEDTAVYYCARKPPHYYVNSFDYWGQGTTVTVSS

SEQ ID NO: 43 - protein, 121 aa, Artificial Sequence
  Synthetic construct
QVHLVQSGSELKKPGASVKISCKASGYTFTTYGMSWVRQAPGQGLEWMGWINTYSGAPTYADDFKGRFVFSLDTSVTTSYLQISTLKAEDTAVYFCARKPPHYYVNSFDYWGQGTLVTVSS

SEQ ID NO: 44 - protein, 121 aa, Artificial Sequence
  Synthetic construct
QVQLVQSGSELKKPGASVKISCKASGYTFTTYGMSWMRQAPGQGLKWMGWINTYSGAPTYADDFKGRFVFSLDTSVNTAFLQISSLQAEDTAVYYCARKPPHYYVNSFDYWGQGTTVTVSS

SEQ ID NO: 45 - protein, 121 aa, Artificial Sequence
  Synthetic construct
QVQLVQSGPEVKKPGASVKVSCKTSGYTFTTYGMSWVRQAPGQGLEWMGWINTYSGAPTYADDFKGRVTMTTDTSTSTAYLELTGLMSDDTAVYFCARKPPHYYVNSFDYWGQGTTVTVSS

SEQ ID NO: 46 - protein, 121 aa, Artificial Sequence
  Synthetic construct
QVQLVQSGPEVKKPGASVKVSCKASGYTFTTYGMSWVRQAPGQGLEWMGWINTYSGAPTYADDFKGRVTITRDTPTSAVYLDLRSLRSDDTAVYYCARKPPHYYVNSFDYWGQGTLVTVSS

SEQ ID NO: 47 - protein, 121 aa, Artificial Sequence
  Synthetic construct
QVQLVQSGAEVKKPGASVKVSCKASGYTFTTYGMSWVRQAPGQRLEWMGWINTYSGAPTYADDFKGRVTITRDTSASTAYMELSSLRSEDTAVYYCARKPPHYYVNSFDYWGQGTLVTVSS

SEQ ID NO: 48 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIK

SEQ ID NO: 49 - protein, 107 aa, Artificial Sequence
  Synthetic construct
EIVLTQSPATLSLSPGERATLSCRASQSISDYLHWYQQKPGQAPRLLIHYASQSISGIPARFSGSGSGTDFTLTISSLEPEDFAVYYCQNGHSFPYTFGGGTKVEIK

SEQ ID NO: 50 - protein, 107 aa, Artificial Sequence
  Synthetic construct
EIVLTQSPDFQSVTPKEKVTITCRASQSISDYLHWYQQKPDQSPKLLIKYASQSISGVPSRFSGSGSGTDFTLTINSLEAEDAATYYCQNGHSFPYTFGQGTKVEIK

SEQ ID NO: 51 - protein, 107 aa, Artificial Sequence
  Synthetic construct
DIVMTQSPSSLSASVGDRVTITCRASQSISDYLHWYQQKPGKAPKLLIYYASQSISGVPSRFSGSGSGTDFTLTISSLQPEDFATYYCQNGHSFPYTFGGGTKVEIK

SEQ ID NO: 52 - protein, 107 aa, Artificial Sequence
  Synthetic construct
EIVMTQSPATLSVSPGERATLSCRASQSISDYLHWYQQKPGEAPRLLIYYASQSISGIPARFSGSGSGTDFTLTISSLEPEDFAVYYCQNGHSFPYTFGQGTKLEIK

SEQ ID NO: 53 - protein, 118 aa, Mus musculus
QVQLQQSGAELARPGASVKLSCKASGYSFTNYGISWVKQRTGQGLEWIGEIYLRSGNNYYNEKFKGKATLTGDKSSSTAYMELRSLTSEDSAVYFCAIYGNYFYFDYWGQGTTLTVSS

SEQ ID NO: 54 - protein, 107 aa, Mus musculus
DIQMTQSPSSLSASLGERVSLTCRASQDIGSSLNWLQQEPDGTIKRLIYATSSLDSGVPKRFSGSRSGSDYSLTISSLESEDFVDYYCLQYASSPFTFGSGTKLEIK

SEQ ID NO: 55 - protein, 5 aa, Artificial Sequence
  Synthetic construct
TYGMS

SEQ ID NO: 56 - protein, 17 aa, Artificial Sequence
  Synthetic construct
WINTYSGAPTYADDFKG

SEQ ID NO: 57 - protein, 12 aa, Artificial Sequence
  Synthetic construct
KPPHYYVNSFDY

SEQ ID NO: 58 - protein, 11 aa, Artificial Sequence
  Synthetic construct
RASQSISDYLH

SEQ ID NO: 59 - protein, 5 aa, Artificial Sequence
  Synthetic construct
GYGVT

SEQ ID NO: 60 - protein, 9 aa, Artificial Sequence
  Synthetic construct
QNGHSFPYT

SEQ ID NO: 61 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 107 aa, Homo sapiens [sequence omitted]

SEQ ID NO: 63 - protein, 416 aa, Artificial Sequence
  Synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 416 aa, Artificial Sequence
  Synthetic construct
SEVEYRAEVGQNAYLPCFYTLPTSGTLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 416 aa, Artificial Sequence
  Synthetic construct
SEVEYRAEVGQNAYLPCFYTPAAPGNLVPVCWGKGACPWSQCGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 416 aa, Artificial Sequence
  Synthetic construct
SEVEYRAEVGQNAYLPCFYTPAAPGNLVPVCWGKGACPVFECGNVVLRTDERNVTYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 416 aa, Artificial Sequence
  Synthetic construct
SEVEYRAEVGQNAYLPCFYTPAAPGNLVPVCWGKGACPVFECGNVVLRTDERDVNYWTSRYWLNGDLNKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 416 aa, Artificial Sequence
  Synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQFPGLMNDKKFNLKLVIKPAKVTPAPTRQRDFTAAFPRMLTTRGHGPAETQTLGSLPDINLTQISTLANELRDSRLANDLRDSGATIRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 346 aa, Artificial Sequence
  Synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 114 aa, Mus musculus
QIQLQQSGPELVTPGTSVKISCKASGYTFTDYYINWVKQRPGQGLEWIGWIYPGSGNTKYNEKFKGKATLTVDTSSSIAYMHLSSLTSEDSAVYFCAGDRFDYWGQGTTLTVSS

SEQ ID NO: 71 - protein, 108 aa, Mus musculus
QIVLTQSPAIMSASPGEKVTLTCSASSGVSSSYLYWYQQKPGSSPKLWIYSTSNLASGVPARFSGSGSGTSYSLTISNMETEDAASYFCHQWSNSPYTFGGGTKLEIK

SEQ ID NO: 72 - protein, 124 aa, Mus musculus
DVQFQESGPGLVKPSQSLSLTCSVTGYSITSGYYWNWIRQFPGNKLEWMGYISYDGSNNYNPSLKNRISITRDTSKNQFFLKLNSVTTEDTATYYCGRDGPYYYGSSYGYFDVWGTGTTVTVSS

SEQ ID NO: 73 - protein, 112 aa, Mus musculus
DIVMTQAESSVPVTPGESVSISCRSSKSLLHSNGNTYLYWFLQRPGQSPQLLIHRMSNLASGVPDRFSGSGSGTAFTLRISRVEAEDMGVYYCMQHLEYPCTFGGGTKLEIK

SEQ ID NO: 74 - protein, 26 aa, Artificial Sequence
  Synthetic construct
RTDERDVNYWTSRYWLNGDFRKGDVS

SEQ ID NO: 75 - protein, 20 aa, Artificial Sequence
  Synthetic construct
DERDVNYWTSRYWLNGDFRK

SEQ ID NO: 76 - protein, 9 aa, Artificial Sequence
  Synthetic construct
RTDERDVNY

SEQ ID NO: 77 - protein, 6 aa, Artificial Sequence
  Synthetic construct
DERDVN

SEQ ID NO: 78 - protein, 10 aa, Artificial Sequence
  Synthetic construct
NGDFRKGDVS

SEQ ID NO: 79 - protein, 4 aa, Artificial Sequence
  Synthetic construct
DFRK

SEQ ID NO: 80 - protein, 16 aa, Artificial Sequence
  Synthetic construct
MIWGDGNTDYNSGLKS

SEQ ID NO: 81 - protein, 9 aa, Artificial Sequence
  Synthetic construct
SYYYGPPDY

SEQ ID NO: 82 - protein, 6 aa, Artificial Sequence
  Synthetic construct
SGYYWN

SEQ ID NO: 83 - protein, 16 aa, Artificial Sequence
  Synthetic construct
YISYDGSNNYNPSLKN

SEQ ID NO: 84 - protein, 15 aa, Artificial Sequence
  Synthetic construct
DGPYYYGSSYGYFDV

SEQ ID NO: 85 - protein, 16 aa, Artificial Sequence
  Synthetic construct
RSSKSLLHSNGNTYLY

SEQ ID NO: 86 - protein, 7 aa, Artificial Sequence
  Synthetic construct
RMSNLAS

SEQ ID NO: 87 - protein, 9 aa, Artificial Sequence
  Synthetic construct
MQHLEYPCT

SEQ ID NO: 88 - protein, 17 aa, Artificial Sequence
  Synthetic construct
KSSQSLLNSRSQKNYLA

SEQ ID NO: 89 - protein, 7 aa, Artificial Sequence
  Synthetic construct
FASTRES

SEQ ID NO: 90 - protein, 5 aa, Artificial Sequence
  Synthetic construct
DYYIN

SEQ ID NO: 91 - protein, 5 aa, Artificial Sequence
  Synthetic construct
NYGMS

SEQ ID NO: 92 - protein, 5 aa, Artificial Sequence
  Synthetic construct
DRFDY

SEQ ID NO: 93 - protein, 12 aa, Artificial Sequence
  Synthetic construct
SASSGVSSSYLY

SEQ ID NO: 94 - protein, 7 aa, Artificial Sequence
  Synthetic construct
STSNLAS

SEQ ID NO: 95 - protein, 9 aa, Artificial Sequence
  Synthetic construct
HQWSNSPYT

SEQ ID NO: 96 - protein, 10 aa, Artificial Sequence
  Synthetic construct
WIYPGSGNTK

SEQ ID NO: 97 - protein, 5 aa, Artificial Sequence
  Synthetic construct
NHGMS

SEQ ID NO: 98 - protein, 10 aa, Artificial Sequence
  Synthetic construct
TISSGGSNTY

SEQ ID NO: 99 - protein, 203 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGGPAETQTLGSLPDINLTQISTLANELRDSRLANDLRDSGATIRIG

SEQ ID NO: 100 - protein, 194 aa, Mus musculus
MFSGLTLNCVLLLLQLLLARSLEDGYKVEVGKNAYLPCSYTLPTSGTLVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGGSETQTLVTLHNNNGTKISTWADEIKDSGETIRTA

SEQ ID NO: 101 - protein, 11 aa, Homo sapiens
AKVTPATTRQT

SEQ ID NO: 102 - protein, 12 aa, Homo sapiens
GEWTGFACHLYE

(19) United States
(12) Patent Application Publication
(10) Pub. No.: US 2016/0200815 A1
(43) Pub. Date: Jul. 14, 2016

(54) ANTIBODIES THAT INHIBIT TIM-3:LILRB2 INTERACTIONS AND USES THEREOF

(71) Applicant: JOUNCE THERAPEUTICS, INC., Cambridge, MA (US)

(72) Inventors: Igor FELDMAN, Jamaica Plain, MA (US); Tatiana NOVOBRANTSEVA, Wellesley, MA (US); Jamie WONG, Cambridge, MA (US); Ryan PHENNICIE, North Reading, MA (US); Stephen Sazinsky, Melrose, MA (US); Sriram Sathyanarayanan, Lexington, MA (US)

(21) Appl. No.: 14/987,703

(22) Filed: Jan. 4, 2016

Related U.S. Application Data
(60) Provisional application No. 62/100,024, filed on Jan. 5, 2015.
(60) Provisional application No. 62/141,794, filed on Apr. 1, 2015.
(60) Provisional application No. 62/256,054, filed on Nov. 16, 2015.

(51) Int. Cl. C07K 16/28

(52) U.S. Cl. CPC C07K 16/2803; C07K 16/2896; C07K 2317/76; C07K 2317/52; C07K 2317/565; C07K 2317/56; C07K 2317/75; C07K 2317/92

(57) ABSTRACT

Provided herein are embodiments relating to therapeutic applications of antibodies that modulate; e.g., inhibit the interaction of TIM-3 and LILRB2 antibodies. In some embodiments, the antibodies bind TIM-3. In some embodiments the antibodies bind LILRB2. Modulation of the interaction of TIM-3 and LILRB2 stimulates the release of pro-inflammatory cytokines; e.g., myeloid-associated pro-inflammatory cytokines.

### CROSS-REFERENCE TO RELATED APPLICATIONS

This application claims priority to U.S. Provisional Patent Application No. 62/100,024, filed Jan. 5, 2015, U.S. Provisional Patent Application No. 62/141,794, filed Apr. 1, 2015, and U.S. Provisional Patent Application No. 62/256,054, filed Nov. 16, 2015; the disclosure of each of which is hereby incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 739512000100SEQLIST.TXT, date recorded: Jan. 4, 2016, size: 100 KB).

### FIELD OF THE INVENTION

The present invention relates to methods of using antibodies that modulate the interaction of TIM-3 and LILRB2 for treating TIM-3 related disorders. Such methods include, but are not limited to, methods of treating cancer.

### BACKGROUND

According to the World Health Organization, cancer is a global pandemic that causes nearly 7 million deaths each year worldwide. That number is expected to reach 10 million by the year 2020. Traditionally, cancer is treated using a variety of modalities including surgery, radiation therapy, and chemotherapy. The choice of treatment depends upon the type, location, and dissemination of the cancer. However, these modalities have proven to be relatively ineffective.

Macrophages undergo specific differentiation depending on the local tissue environment. Two distinct states of polarized activation for macrophages have been defined: the classically activated (M1) macrophage phenotype and the alternatively activated (M2) macrophage phenotype (Gordon and Taylor, 2005. Nat. Rev. Immunol. 5:953-964; Mantovani et al., 2002. Trends Immunol. 23:549-555) that can be distinguished by surface marker expression, cytokine production and their specific functional activities (Biswas and Mantovani, 2010. Nat. Immunol. 11:889-896). Classically activated (M1) macrophages have a pro-inflammatory profile. The alternatively activated (M2) macrophages appear to be involved in immunosuppression and tissue repair.

LPS and the T_{H}1 cytokine IFNγ polarize macrophages towards the M1 phenotype which induces the macrophage to produce large amounts of TNF, IL-12, and IL-23. This helps to drive antigen specific T_{H}1 and T_{H}17 cell inflammatory responses. The antimicrobial functions of M1 macrophages are linked to up-regulation of enzymes, such as inducible nitric oxide synthase (iNOS) that generates nitric oxide from L-arginine.

In contrast, exposure of macrophages to the T_{H}2 cytokine IL-4 produces a M2 phenotype which induces the production of high levels of IL-10 and IL-1RA and low expression of IL-12. These cells help with parasite clearance, reduce inflammation, are immunoregulators, promote tissue remodeling and tumor progression. M2 macrophages also express high levels of scavenger mannose and galactose receptors.

M2 macrophages can be further divided into subsets: M2a, M2b, and M2c based on gene expression profiles. The M2a subtype is elicited by IL-4 or IL-13. The M2b is elicited by IL-1R ligands or exposure to immune complexes plus LPS. The M2c subtype by IL-10, TGF-β and glucocorticoid hormones.

The T-cell immunoglobulin mucin (TIM) family regulates T-cell activation and tolerance. See Kane, L. P. T Cell Ig and Mucin Domain Proteins and Immunity, J Immunol. (2010) 184:2743-2749; Freeman et al., TIM genes: a family of cell surface phosphatidylserine receptors that regulate innate and adaptive immunity, Immunol Rev (2010) 235:172-89; and Zhu, C. TIM-3 and its regulatory role in immune responses. Curr Top Microbiol Immunol. (2009) 350:1-15. There are eight predicted tim genes in the murine genome (on mouse chromosome 11B1.1), four of which are known to encode 4 known functional proteins: TIM-1 (T-cell immunoglobulin and mucin domain-containing protein 1 or Hepatitis A virus cellular receptor 1/HAVCR1 homolog), TIM-2 (T-cell immunoglobulin and mucin domain-containing protein 2/TIMD-2), TIM-3 (T-cell immunoglobulin and mucin domain-containing protein 3 or Hepatitis A virus cellular receptor 2/HAVCR2 homolog) and TIM-4 (T-cell immunoglobulin and mucin domain-containing protein 4/TIMD-4), as well as four putative proteins TIM-5, TIM-6, TIM-7 and TIM-8. In contrast to mice, the human genome (on human chromosome 5q33.2) contains only three TIM genes, all encoding functional proteins, TIM-1 (HAVCR1), TIM-3 (HAVCR2) and TIM-4. See Kane, L. P. T Cell Ig and Mucin Domain Proteins and Immunity, J Immunol. (2010) 184:2743-2749. TIM family members are expressed on a wide variety of innate and adaptive immune cells and have been implicated in regulating normal immune responses, and in diseases like autoimmunity, cancer and asthma. See Kuchroo V. J. et al., TIM family of genes in immunity and tolerance. Adv Immunol. (2006) 91:227-49; Kane, L. P. Immune regulation by the TIM Gene family Immunologic Research (2006) 36(1-3):147-155; Kane, L. P. T Cell Ig and Mucin Domain Proteins and Immunity, J Immunol. (2010) 184:2743-2749; and Zhu, C. TIM-3 and its regulatory role in immune responses. Curr Top Microbiol Immunol. (2009) 350:1-15.

TIM family members also belong to the immunoglobulin superfamily Members of the TIM family are type I transmembrane proteins, and contain a characteristic N-terminal immunoglobulin-V-like (IgV) domain, a mucin domain with O-linked glycosylation sites, membrane proximal N-linked glycosylation sites, a single transmembrane domain, and a cytoplasmic region with tyrosine kinase phosphorylation motif(s) (except TIM-4 which does not have a tyrosine kinase phosphorylation motif in its cytoplasmic region). The length of the mucin domain is variable, and depends on the family member, with TIM-3 bearing the shortest length. See Freeman, G. J. TIM genes: a family of cell surface phosphatidylserine receptors that regulate innate and adaptive immunity. Immunological Reviews (2010) 235:172-189; Kane, L. P. Immune regulation by the TIM Gene family, Immunologic Research (2006) 36(1-3):147-155; Kane, L. P. T Cell Ig and Mucin Domain Proteins and Immunity, J Immunol. (2010) 184:2743-2749 and Zhu, C. TIM-3 and its regulatory role in immune responses. Curr Top Microbiol Immunol. (2009) 350:1-15. The N-terminal IgV domain has a deep binding pocket (called the metal ion-dependent ligand-binding site (MILIBS)) that is flanked by two hydrophobic loops which extend to the membrane. The IgV domain is composed of two anti-parallel β-sheets with particularly short β-strands. See Freeman, G. J. et al., TIM genes: a family of cell surface phosphatidylserine receptors that regulate innate and adaptive immunity. Immunological Reviews (2010) 235:172-189. This domain also possess six invariant cysteines, two (the first and sixth cysteines) of which form disulphide bonds bridging the two β-sheets, as in all immunoglobulin superfamily members. See Cao, E. et al. T cell immunoglobulin Mucin-3 crystal structure reveals a galactin-9-independent ligand-binding surface. Immunity (2007) 26:311-321. Without wishing to be bound by theory, these bonds stabilize the IgV domain of TIM-3 and reorient the CC′ loop so that it is in close proximity to the FG loop resulting in formation of a “cleft” or “pocket” structure in TIM-3 as well as other TIM proteins. This unique cleft structure is not found in other IgSF proteins and has been predicted to be involved in ligand binding. In the cytoplasmic region of both human and mouse TIM-3, there is a highly conserved region containing five tyrosine residues. Galectin-9 binding to TIM-3 results in tyrosine phosphorylation of these residues, indicating that some, if not all, of these tyrosines may be involved in TIM-3 signaling. Otherwise, protein sequence analysis does not reveal any other homology to known inhibitory domains such as an immunoreceptor tyrosine-based inhibitory motif or immunoreceptor tyrosine-based switch motif. See Zhu, C. et al., TIM-3 and Its regulatory role in immune responses. Curr Top Microbiol Immunol (2011) 350:1-15.

TIM-3 differs both structurally and in terms of spatial expression patterns from other TIM family members, which suggests that it might have distinct functions compared to other TIM family members. For example, whereas TIM-1 is expressed exclusively on T-helper 2 (Th2) cells, and TIM-4 is expressed on antigen presenting cells (APC), TIM-3 is expressed on T-helper 1 (Th1) cells, T-helper 17 (Th17) cells, IFN-γ producing CD8+ cytotoxic T 1 (Tc1) cells, as well as on dendritic cells (DC), macrophages, natural killer (NK) cells, natural killer T (NKT) cells and human monocytes. When present on DC, TIM-3 mediates uptake of apoptotic cells. TIM-3 expression is regulated by T-bet, a Th1 transcription factor. See Freeman, G. J. et al., TIM genes: a family of cell surface phosphatidylserine receptors that regulate innate and adaptive immunity Immunological Reviews (2010) 235:172-189. TIM-3 is hypothesized to be a negative regulator of T cell responses. For example, binding of TIM-3 to its putative ligand, galectin-9, on Th1 cells, results in Th1 cell death. Further, blockade of TIM-3 increases IFN-γ secreting T cells. See Zhu, C. et al. The TIM-3 ligand galactin-9 negatively regulates T helper type 1 immunity. Nat Immunol. (2005) 6:1245-1252 and Sabatos, C. A. et al. Interaction of TIM-3 and TIM-3 ligand regulated T helper type 1 responses and induction of peripheral tolerance. Nat. Immunol. (2003) 4:1102-1110. Additionally, co-blockade of TIM-3 and another of its putative ligand, CEACAM1, leads to enhancement of anti-tumor immune responses with improved elimination of tumors in mouse colorectal cancer models. See Huang, et al. CEACAM1 regulates TIM-3-mediated tolerance and exhaustion. Nature (2014).

Several ligands and/or co-receptors for TIM-3 have been identified, including HMGB1, Galectin 9 and phosphatidylserine. See Hang Li et al., TIM-3/galectin-9 signaling pathway mediates T-cell dysfunction. Hepatology (2012) 56(4):1342-1351, Shigeki, K et al., Galectin-9 inhibits CD44-hyluronan interaction and suppresses a murine model of allergic asthma. Am L Respir Crit Care Med (2007) 176:27-35; Kang, R. et al., HMGB1 in Cancer. Clin Cancer Res (2013) (PMID: 23723299), Kane, L. P. T cell Ig and mucin domain proteins and immunity. J Immunol (2010) 184:2743-2749, and Zhu, C. et al., TIM-3 and its regulatory role in immune responses. Curr Top Microbiol Immunol (2011) 350:1-15. Chiba, S., et al., Tumor-infiltrating DCs suppress nucleic acid-mediated innate immune responses through interactions between the receptor TIM-3 and the alarmin HMGB1, Nat. Immunol (2012) 13(9):832-842.

Given TIM-3's negative regulation of T cell responses, TIM-3 was initially hypothesized to regulate antitumor responses, and exploited by tumors to evade immune clearance. See Ngiow, S. F. et al. Prospects for TIM-3-targeted anti-tumor Immunotherapy. Cancer Research. (2011) 71:6567-6571. However, subsequent studies showed that TIM-3 expression on innate cells contributed to pro-inflammatory responses. See Leavy O. TIM-3: dual role in immunity. Nature Reviews Immunology (2008) 8:4; and Anderson, A. C. et al., Promotion of tissue inflammation by the immune receptor TIM-3 expressed on innate immune cells Science (2007) 318(5853):1141-1143. On innate cells, where TIM-3 is expressed constitutively in both humans and mice, TIM-3 synergizes with Toll-like receptors (TLR) and promotes Th1 immunity, by increasing the production of pro-inflammatory cytokines by DCs. This disparate and dual functionality of TIM-3 is hypothesized to occur as a result of differences in TIM-3 expression, with inhibitory functions attributed to its expression on T cells, and stimulatory/pro-inflammatory functions attributed to its expression on innate cells. It is also hypothesized that differences in the proximal signaling pathways induced by TIM-3 might account for the differences in TIM-3's effect on innate and adaptive immune cells. Thus, TIM-3 has been implicated in either promoting or terminating Th1 immunity, and without being bound by theory, has paradoxical roles in modulating immune responses by providing costimulatory and/or coinhibitory signals. See Anderson, A. C. et al., Promotion of tissue inflammation by the immune receptor TIM-3 expressed on innate immune cells Science (2007) 318(5853):1141-1143.

TIM-3 is hypothesized to have paradoxical roles in modulating immune responses by providing costimulatory or coinhibitory signals depending on its binding to different receptors and/or its spatial expression on different immune cells. For example, blockade of TIM-3 signaling during induction of experimental autoimmune encephalitis leads to macrophage expansion and activation resulting in a more severe clinical phenotype. See Monney et al., Th1-specific cell surface protein TIM-3 regulates macrophage activation and severity of an autoimmune disease. (2002) Nature 415:536-541; and Anderson, D. E. Expert Opin Ther Targets. (2007) August; 11(8):1005-9. In contrast, TIM-3 also acts synergistically with Toll-like receptors to increase pro-inflammatory TNFα secretion from dendritic cells, which may in turn promote T effector responses. See Anderson et al., Promotion of tissue inflammation by the immune receptor TIM-3 expressed on innate immune cells. (2007) Science 318:1141-1143. Thus, TIM-3 has been implicated in either promoting or terminating Th1 immunity.

Although the biological role of TIM-3 signaling in T cell activation and in modulating immune responses is still being unraveled, it is clear that TIM-3 is an important target in cancer therapy. There remains a need for more effective treatments of cancer utilizing immunotherapy. More particularly, there remains a need for novel anti-TIM-3 antibodies, compositions and therapeutic agents, and methods comprising the same, that modulate TIM-3 activity which are capable of enhancing the host immune response against tumors for treating cancer.

### SUMMARY OF THE INVENTION

The invention provides antibodies which modulate the interaction of TIM-3 and LILRB2. In some embodiments, the antibody inhibits the interaction of TIM-3 and LILRB2. In some embodiments, the antibody inhibits the binding of TIM-3 to LILRB2.

In some aspects, the invention provides antibodies which specifically bind TIM-3, wherein the antibodies modulate the interaction of TIM-3 and LILRB2. In some embodiments, binding of the antibody to TIM-3 inhibits the interaction of TIM-3 to LILRB2. In some embodiments, binding of the antibody to TIM-3 inhibits binding of TIM-3 to LILRB2. In some embodiments, binding of the antibody to TIM-3 inhibits binding of TIM-3 to LILRB2 by at least about any of 1%, 5%, 10%, 20%, 30%, 40%, 50%, 60%, 70%, 80%, 90%, or 100%. In some embodiments, the antibody competes with LILRB2 for binding to TIM-3. In some embodiments, binding of the antibody to TIM-3 competes with LILRB2 for binding of TIM-3 to LILRB2 where the binding of TIM-3 to LILRB2 is reduced by at least about any of 1%, 5%, 10%, 20%, 30%, 40%, 50%, 60%, 70%, 80%, 90%, or 100%.

In some aspects, the invention provides antibodies which specifically bind LILRB2, wherein the antibodies modulate the interaction of LILRB2 and TIM-3. In some embodiments, binding of the antibody to LILRB2 inhibits the interaction of LILRB2 to TIM-3. In some embodiments, binding of the antibody to LILRB2 inhibits binding of LILRB2 to TIM-3. In some embodiments, binding of the antibody to LILRB2 inhibits binding of LILRB2 to TIM-3 by at least about any of 1%, 5%, 10%, 20%, 30%, 40%, 50%, 60%, 70%, 80%, 90%, or 100%. In some embodiments, the antibody competes with TIM-3 for binding to LILRB2. In some embodiments, binding of the antibody to LILRB2 competes with TIM-3 for binding of LILRB2 to TIM-3 where the binding of LILRB2 to TIM-3 is reduced by at least about any of 1%, 5%, 10%, 20%, 30%, 40%, 50%, 60%, 70%, 80%, 90%, or 100%.

In some embodiments of any of the above embodiments, the TIM-3 is human TIM-3. In some embodiments, the TIM-3 comprises the amino acid sequence of SEQ ID NO:1 or SEQ ID NO:3. In other embodiments, the amino acid sequence of the TIM-3 is at least about 80% identical to the amino acid sequence set forth in SEQ ID NO:1 or SEQ ID NO:3.

In some embodiments of any of the above embodiments, the LILRB2 is human LILRB2. In some embodiments, the LILRB2 comprises the amino acid sequence of SEQ ID NO:5 or SEQ ID NO:7. In other embodiments, the amino acid sequence of the LILRB2 is at least about 80% identical to the amino acid sequence set forth in SEQ ID NO:5 or SEQ ID NO:7.

In some embodiments, the antibody of the invention competes with antibody mAb13, mAb15, mAb17, mAb22, mAb48, mAb58 and/or mAb91 for binding human TIM-3. In some embodiments, the antibody of the invention competes with antibody mAb13, mAb15, mAb17, mAb22, mAb48, mAb58 and/or mAb91 for binding human TIM-3 and stimulates the secretion of one or more myeloid-associated cytokines in an individual; for example, increases the secretion of one or more myeloid-associated cytokines in an individual. In some embodiments, the myeloid associated cytokine is one or more of IL-2, TNFα, IL-1β, GM-CSF or IL-6. In some embodiments, the myeloid associated cytokine is one or more of TNFα, IL-1β or IL-6. In some embodiments, the myeloid associated cytokines are TNFα, IL-1β and IL-6. In some embodiments, the antibody stimulates the secretion of a myeloid-associated cytokine in an individual to a greater extent than the stimulation of secretion of the cytokine by antibody F38-2E2. In some embodiments, the antibody stimulates the secretion (e.g., increases the secretion) of a myeloid-associated cytokine in an individual to greater than about any one of 5%, 10%, 15%, 20%, 25%, 30%, 40%, 50%, 60%, 70%, 80%, 90%, or 100% the stimulation of secretion of the cytokine by antibody F38-2E2. In some embodiments, the antibody suppresses the secretion of a myeloid-associated cytokine in an individual. In some embodiments, secretion of myeloid associated cytokine IL-10, CCL2, CCL3, CCL4 or CCL5 is suppressed. In some embodiments, secretion of IL-10 is suppressed. In some embodiments, secretion of CCL2 is suppressed. In some embodiments, secretion of CCL3 is suppressed. In some embodiments, secretion of CCL4 is suppressed. In some embodiments, secretion of CCL5 is suppressed. In some embodiments, the antibody suppresses the secretion of a myeloid-associated cytokine in an individual to a greater extent than the suppression of secretion of the cytokine by antibody F38-2E2. In some embodiments, the antibody suppresses the secretion of a myeloid-associated cytokine in an individual to greater than about any one of 5%, 10%, 15%, 20%, 25%, 30%, 40%, 50%, 60%, 70%, 80%, 90%, or 100% the suppression of secretion of the cytokine by antibody F38-2E2.

In some aspects, the invention provides an antibody that binds TIM-3, wherein the antibody stimulates the secretion of one or more myeloid-associated cytokines in an individual; for example, increases the secretion of one or more myeloid-associated cytokines. In some embodiments, the myeloid associated cytokine is one or more of IL-12, TNFα, IL-1β, GM-CSF or IL-6. In some embodiments, the myeloid associated cytokine is one or more of TNFα, IL-1β, or IL-6. In some embodiments, the myeloid associated cytokines are TNFα, IL-1β, and IL-6. In some embodiments, the antibody stimulates the secretion of one or more myeloid-associated cytokines in an individual to a greater extent than the stimulation of secretion of the cytokine by antibody F38-2E2. In some embodiments, the antibody stimulates the secretion (e.g., increases the secretion) of a myeloid-associated cytokine in an individual to greater than about any one of 5%, 10%, 15%, 20%, 25%, 30%, 40%, 50%, 60%, 70%, 80%, 90%, or 100% the stimulation of secretion of the cytokine by antibody F38-2E2. In some embodiments, the antibody suppresses the secretion of a myeloid-associated cytokine in an individual; for example, decreases the secretion of a myeloid-associated cytokine. In some embodiments, secretion of myeloid associated cytokine IL-10, CCL2, CCL3, CCL4 or CCL5 is suppressed. In some embodiments, secretion of IL-10 is suppressed. In some embodiments, secretion of CCL2 is suppressed. In some embodiments, secretion of CCL3 is suppressed. In some embodiments, secretion of CCL4 is suppressed. In some embodiments, secretion of CCL5 is suppressed. In some embodiments, the antibody suppresses the secretion of one or more myeloid-associated cytokines in an individual to a greater extent than the suppression of secretion of the cytokine by antibody F38-2E2. In some embodiments, the antibody suppresses the secretion of a myeloid-associated cytokine in an individual to greater than about any one of 5%, 10%, 15%, 20%, 25%, 30%, 40%, 50%, 60%, 70%, 80%, 90%, or 100% the stimulation of secretion of the cytokine by antibody F38-2E2. In some embodiments, the antibody competes with mAb13, mAb15, mAb17, mAb22, mAb48, mAb58 and/or mAb91 for binding TIM-3 (e.g., human TIM-3).

In some aspects, the invention provides an antibody that binds an epitope of TIM-3 such that secretion of one or more myeloid-associated cytokines is stimulated in an individual; for example, increases the secretion of one or more myeloid-associated cytokines. In some embodiments, the myeloid-associated cytokine is one or more of IL-12, TNFα, IL-1β, GM-CSF, or IL-6. In some embodiments, the myeloid-associated cytokine is one or more of TNFα, IL-1β or IL-6. In some embodiments, the myeloid-associated cytokines are TNFα, IL-1β and IL-6. In some embodiments, binding of the antibody to an epitope of TIM-3 preferentially stimulates the secretion of cytokines from macrophages. In some embodiments, binding of the antibody to an epitope of TIM-3 suppresses the secretion of one or more myeloid-associated cytokines in an individual. In some embodiments, binding of the antibody to an epitope of TIM-3 reduces the secretion of one or more myeloid-associated cytokines in an individual. In some embodiments, secretion of one or more of myeloid associated cytokines IL-10, CCL2, CCL3, CCL4 or CCL5 is suppressed by binding of the antibody to an epitope of TIM-3. In some embodiments, secretion of one or more of myeloid associated cytokines IL-10, CCL2, CCL3, CCL4 or CCL5 is reduced by binding of the antibody to an epitope of TIM-3. In some embodiments, binding of the antibody to an epitope of TIM-3 stimulates secretion of proinflammatory cytokines and/or inhibits secretion of immune suppressor cytokines. In some embodiments, binding of the antibody to an epitope of TIM-3 stimulates macrophages of an M1 phenotype and reduces macrophages of an M2 phenotype. In some embodiments, the individual has cancer. In some embodiments, the cytokine is secreted in a tumor. In some embodiments, the individual is a human.

In some embodiments of the any of the above embodiments, the antibody is a monoclonal antibody. In some embodiments, the antibody is a chimeric antibody. In other embodiments, the antibody is humanized. In yet other embodiments, the antibody is a human antibody. In some embodiments, the antibody is an antigen binding fragment of an antibody. In some embodiments, the antibody is an antibody fragment selected from a Fab, Fab′, Fv, scFv or (Fab′)2 fragment.

In some aspects, the invention provides a pharmaceutical composition comprising the antibody of any the above embodiments and a pharmaceutically acceptable carrier.

In some aspects, the invention provides methods of stimulating the secretion of one or more myeloid-associated cytokines in an individual, comprising administering to the individual a therapeutically effective amount of an antibody that inhibits the interaction of TIM-3 and LILRB2. In some embodiments, the antibody is in a pharmaceutical composition. In some embodiments, the myeloid-associated cytokine is one or more of IL-12, TNFα, IL-1β, GM-CSF, or IL-6. In some embodiments, the myeloid-associated cytokine is one or more of TNFα, IL-1β or IL-6. In some embodiments, the myeloid-associated cytokines are TNFα, IL-1β and IL-6. In some embodiments, administration of the antibody to the individual preferentially stimulates the secretion (e.g., increases the secretion) of cytokines from macrophages. In some embodiments, administration of the antibody suppresses the secretion of one or more myeloid-associated cytokines in an individual. In some embodiments, administration of the antibody reduces the secretion of one or more myeloid-associated cytokines in an individual. In some embodiments, secretion of one or more of myeloid associated cytokines IL-10, CCL2, CCL3, CCL4 or CCL5 is suppressed by administration of the antibody. In some embodiments, the individual has cancer. In some embodiments, the cytokine is secreted in a tumor. In some embodiments, the individual is a human.

In some aspects, the invention provides methods for treating cancer in an individual, comprising administering to the individual a therapeutically effective amount of the antibody as described herein. In some embodiments, the antibody is in a pharmaceutical composition. In some embodiments, the individual is a human.

In some embodiments, the invention provides an isolated nucleic acid encoding an antibody that inhibits the interaction of TIM-3 and LILRB2 as described herein. In some embodiments, the invention provides a vector comprising the nucleic acid encoding the antibody. In some embodiments, the invention provides a host cell comprising the nucleic acid or the vector. In some embodiments, the invention provides a host cell that produces an antibody as described herein.

In some aspects, the invention provides methods for making an antibody that modulates the interaction of TIM-3 and LILRB2 by culturing a host cell comprising a nucleic acid encoding the antibody under conditions suitable for expression of the nucleic acid encoding the antibody that modulates the interaction of TIM-3 and LILRB2. In some embodiments, the invention provides methods for making an antibody that inhibits the interaction of TIM-3 and LILRB2 by culturing a host cell comprising the nucleic acid encoding the antibody under conditions suitable for expression of the nucleic acid encoding the antibody that inhibits the interaction of TIM-3 and LILRB2. In further embodiments the method further comprises recovering the antibody produced by the host cell.

In some embodiments, the invention provides the use of an antibody that inhibits the interaction of TIM-3 and LILRB2 for stimulating the secretion of one or more myeloid-associated cytokines in an individual in need thereof. In some embodiments, the invention provides the use of an antibody as described herein in the manufacture of a medicament for stimulating the secretion of one or more myeloid-associated cytokines in an individual in need thereof. In some embodiments, the antibody is in a pharmaceutical composition. In some embodiments, the myeloid-associated cytokine is one or more of IL-12, TNFα, IL-1β, GM-CSF or IL-6. In some embodiments, the myeloid-associated cytokine is one or more of TNFα, IL-1β, or IL-6. In some embodiments, the myeloid-associated cytokines are TNFα, IL-1β and IL-6. In some embodiments, the antibody suppresses the secretion of a myeloid-associated cytokine in an individual; for example, reduces secretion of a myeloid-associated cytokine in an individual. In some embodiments, secretion of myeloid associated cytokine IL-10, CCL2, CCL3, CCL4 or CCL5 is suppressed. In some embodiments, secretion of IL-10 is suppressed. In some embodiments, secretion of CCL2 is suppressed. In some embodiments, secretion of myeloid associated cytokine CCL3 is suppressed. In some embodiments, secretion of CCL4 is suppressed. In some embodiments, secretion of CCL5 is suppressed. In some embodiments, the individual has cancer. In some embodiments, the individual is human.

In some embodiments, the invention provides the use of an antibody that inhibits the interaction of TIM-3 and LILRB2 for treating cancer in an individual. In some embodiments, the invention provides the use of an antibody that inhibits the interaction of TIM-3 and LILRB2 in the manufacture of a medicament for treating cancer in an individual. In some embodiments, the antibody is in a pharmaceutical formulation.

In some embodiments, the invention provides a pharmaceutical composition for treating cancer in an individual comprising a therapeutically effective amount of an antibody that modulates the interaction of TIM-3 and LILRB2 as described herein and a pharmaceutically acceptable carrier. In some embodiments, the invention provides a pharmaceutical composition for treating cancer in an individual comprising a therapeutically effective amount of an antibody that inhibits the interaction of TIM-3 and LILRB2 as described herein and a pharmaceutically acceptable carrier.

In some embodiments, the invention provides kits for stimulating the secretion of myeloid-associated cytokines in an individual, comprising the antibody that inhibits the interaction of TIM-3 and LILRB2. In some embodiments, the antibody is in a pharmaceutical formulation. In some embodiments, the invention provides kits for increasing the secretion of myeloid-associated cytokines. In some embodiments, the myeloid-associated cytokine is one or more of IL-12, TNFα, IL-1β, GM-CSF or IL-6. In some embodiments, the myeloid-associated cytokine is one or more of TNFα, IL-1β, or IL-6. In some embodiments, the myeloid-associated cytokines are TNFα, IL-1β and IL-6. In some embodiments, the antibody of the kit reduces the secretion of a myeloid-associated cytokine in an individual. In some embodiments, the antibody of the kit suppresses the secretion of a myeloid-associated cytokine in an individual. In some embodiments, secretion of myeloid associated cytokine IL-10, CCL2, CCL3, CCL4 or CCL5 is suppressed. In some embodiments, secretion of IL-10 is suppressed. In some embodiments, secretion of CCL2 is suppressed. In some embodiments, secretion of myeloid associated cytokine CCL3 is suppressed. In some embodiments, secretion of CCL4 is suppressed. In some embodiments, secretion of CCL5 is suppressed. In some embodiments, the individual has cancer. In some embodiments, the invention provides kits for treating cancer in an individual, comprising the antibody that inhibits the interaction of TIM-3 and LILRB2.

In some aspects, the invention provides methods for screening an agent for the presence or absence of modulation of the interaction of TIM-3 and LILRB2, the method comprising measuring the binding of TIM-3 and LILRB2 in the presence of a candidate agent, wherein a change in the binding of TIM-3 and LILRB2 in the presence of the candidate agent compared to binding of TIM-3 and LILRB2 in the absence of the candidate agent indicates that the agent modulates the interaction of TIM-3 and LILRB2. In some embodiments, the modulation of the interaction of TIM-3 and LILRB2 is an inhibition of the interaction of TIM-3 and LILRB2. In some embodiments, the inhibition of the interaction of TIM-3 and LILRB2 is an inhibition of the binding of TIM-3 and LILRB2. In some embodiments, the change in binding of TIM-3 and LILRB2 is at least about 1%, 5%, 10%, 20%, 30%, 40%, 50%, 60%, 70%, 80%, 90% or 100%. In some embodiments, the agent that inhibits the interaction of TIM-3 and LILRB2 stimulates the secretion of a myeloid-associated cytokine (e.g., increases the secretion of a myeloid-associated cytokine) following administration to an individual. In some embodiments, the agent is an antibody.

In some aspects, the invention provides methods for screening an agent which inhibits the interaction of TIM-3 and LILRB2, the method comprising measuring the binding of TIM-3 and LILRB2 in the presence of a candidate agent, wherein a reduction in the binding of TIM-3 and LILRB2 in the presence of the candidate agent compared to binding of TIM-3 and LILRB2 in the absence of the candidate agent indicates that the agent inhibits the interaction of TIM-3 and LILRB2. In some embodiments, the inhibition of the interaction of TIM-3 and LILRB2 is an inhibition of the binding of TIM-3 and LILRB2. In some embodiments, the reduction in binding of TIM-3 and LILRB2 is at least about 1%, 5%, 10%, 20%, 30%, 40%, 50%, 60%, 70%, 80%, 90% or 100%. In some embodiments, the agent that inhibits the interaction of TIM-3 and LILRB2 stimulates the secretion of a myeloid-associated cytokine (e.g., increases the secretion of a myeloid-associated cytokine) following administration to an individual. In some embodiments, the agent is an antibody.

In some aspects, the invention provides an antibody which specifically binds an epitope of TIM-3, wherein the epitope comprises the C′C″ and DE loop of TIM-3. In some embodiments, the epitope comprises the amino acid sequence RTDERDVNYWTSRYWLNGDFRKGDVS (SEQ ID NO:74). In some embodiments, the epitope comprises the amino acid sequence DERDVNYWTSRYWLNGDFRK (SEQ ID NO:75). In some aspects, the invention provides an antibody which specifically binds an epitope of TIM-3, wherein the epitope comprises the C′C″ loop of TIM-3. In some embodiments, the epitope comprises the amino acid sequence RTDERDVNY (SEQ ID NO:76). In some embodiments, the epitope comprises the amino acid sequence DERDVN (SEQ ID NO:77). In some embodiments, the epitope comprises the amino acid sequence DVN. In some aspects, the invention provides an antibody which specifically binds an epitope of TIM-3, wherein the epitope comprises the DE loop of TIM-3. In some embodiments, the epitope comprises the amino acid sequence NGDFRKGDVS (SEQ ID NO:78). In some embodiments, the epitope comprises the amino acid sequence DFRK (SEQ ID NO:79). In some embodiments, the epitope comprises the amino acid sequence DFR or FRK. In some embodiments of the above-embodiments, the antibody binds the C′C″ and/or DE loop of TIM-3 with greater affinity than the antibody binds the CC′ loop of TIM-3. In some embodiments, the antibody binds the C′C″ and/or DE loop of TIM-3 with greater affinity than antibody F38-2E2 binds the CC′ loop of TIM-3. 102. The antibody of any one of claims 89-101, wherein binding to the antibody to the C′C″ and/or DE loop of TIM-3 stimulates the expression of one or more myeloid-associated cytokines. In some embodiments, the myeloid-associated cytokine is one or more of IL-12, TNFα, IL-1β, GM-CSF or IL-6. In some embodiments, binding to the antibody to the C′C″ and/or DE loop of TIM-3 stimulates proinflammatory macrophages. In some embodiments, binding to the antibody to the C′C″ and/or DE loop of TIM-3 stimulates macrophages of an M1 phenotype. In some embodiments, binding to the antibody to the C′C″ and/or DE loop of TIM-3 suppresses secretion of one or more myeloid-associated cytokines. In some embodiments, the myeloid-associated cytokine is one or more of IL-10, CCL2, CCL3, CCL4 or CCL5. In some embodiments, binding to the antibody to the C′C″ and/or DE loop of TIM-3 reduces immunosuppressive macrophages. In some embodiments, binding to the antibody to the C′C″ and/or DE loop of TIM-3 reduces macrophages of an M2 phenotype. In some embodiments, the TIM-3 is human TIM-3. In some embodiments, the antibody is a monoclonal antibody. In some embodiments, the antibody is a chimeric antibody. In other embodiments, the antibody is humanized. In other embodiments, the antibody is a human antibody. In other embodiments, the antibody is an antibody fragment selected from a Fab, Fab′, Fv, scFv or (Fab′)2 fragment. In some embodiments, the invention provides a pharmaceutical composition comprising the antibody as described herein and a pharmaceutically acceptable carrier.

### BRIEF DESCRIPTION OF THE DRAWINGS

FIG. 1A is a graph showing IL-2 secretion by SEB-activated whole blood samples treated with no antibody, an isotype control antibody, an anti-PD-L1 antibody with an IgG1 isotype control antibody, antibody F38-2E2, or antibody F38-2E2 and anti-PD-L1. ** p<0.01; **** p<0.0001. FIG. 1B. shows diverse bins of anti-TIM-3 antibodies when arranged according to their ability to cross-block one another in binding plate-bound TIM-3 protein.

FIGS. 2A and 2B show that SEB induction of TIM-3 on monocyte/macrophages has different kinetics than on T cells. FIG. 2A is a graph showing a time course of expression of PD-1 on the surface of indicated cells from time=0 to four days in culture. FIG. 2B is a graph showing a time course of expression of TIM-3 on the surface of indicated cells from time=0 to four days in culture. Circles represent CD4+ T cells, squares represent CD8+ T cells, triangles represent CD14+ monocytes/macrophages, and diamonds represent CD11c+ dendritic cells (DCs).

FIGS. 3A-3O show SEB induction of innate inflammatory cytokines and IL-2 can be measured before TIM-3 is upregulated on T cells. SEB-activated PBMC were treated with a control isotype antibody (circles), an anti-PD-L1 antibody (squares), an anti-TIM-3 antibody (triangles) or an anti-PD-L1 antibody and an anti-TIM-3 antibody (inverted triangles). FIG. 3A shows expression of IL-2 over the four day time course. FIG. 3B shows expression of TNFα over the four day time course. FIG. 3C shows expression of IL-1β over the four day time course. FIGS. 3D-3O show the expression of other cytokines as indicated over the four day time course.

FIGS. 4A and 4B show that TIM-3 is more strongly associated with myeloid cells (monocytes/macrophages and dendritic cells) than T cells in human cancers. FIG. 4A and FIG. 4B show graphs representing the correlation of TIM-3 expression and the T cell marker CD3g (FIG. 4A) or the myeloid cell marker CD11b (FIG. 4B) in tumor samples from a breast cancer (BRCA), a lung adenocarcinoma (LUAD), an ovarian cancer (OV), and a prostate adenocarcinoma (PRAD). X and Y axes represent normalized level of mRNA expression, Corr(S) stands for Spearman correlation coefficient, Pval(S) denotes p-value of the correlation. Similar results were seen with tumor samples from a head and neck cancer.

FIGS. 5A-5F show graphs demonstrating that TIM-3 inhibition stimulates expression of DC co-stimulatory molecules and cytokine release by DCs. Following LPS activation, DCs were treated with no antibody, a mIgG1 isotype control, a commercially available anti-TIM-3 antibody (F38-2E2) or antibodies generated as described in Example 1. Co-stimulatory molecules or cytokines were measured on Day 4 post-LPS activation. FIG. 5A shows expression of the co-stimulatory molecule CD80 (B7-1). FIG. 5B shows expression of the co-stimulatory molecule CD86 (B7-2). FIG. 5C shows expression of the cytokine IL-1β. FIG. 5D shows expression of the cytokine TNFα. FIG. 5E shows expression of the cytokine IL-12/IL-23p40 **** p<0.0001. FIG. 5F shows FACS gating for LPS activated MDDCs.

FIG. 6A is a graph showing that human LILRB2 binds human TIM-3. FIG. 6B is a graph showing the high correlation between TIM-3 and LILRB2 transcript levels across tumor samples.

FIGS. 7A-7C are graphs showing that anti-TIM-3 antibodies and anti-LILRB2 antibodies can block the interaction of TIM-3 and LILRB2. FIG. 7A is a composite of the binding data presented in FIGS. 7B and 7C. FIG. 7B shows anti-TIM-3 antibodies block association of human TIM-3 to human LILRB2. FIG. 7C shows anti-LILRB2 antibodies block association of human LILRB2 to human TIM-3. Anti-TIM-3 antibodies were the commercially available antibody F38-2E2 and antibodies mAb5, mAb13, mAb15, mAb21, mAb26, and mAb27 generated as described in Example 1. Anti-LILRB2 antibodies were R&D polyclonal anti-LILRB2, R&D monoclonal anti-LILRB2 (clone 287219), and antibody 42D1. mIgG1 served as an isotype control antibody.

FIGS. 8A and 8B show graphs demonstrating TNFα release from macrophages (FIG. 8A) and from DCs (FIG. 8B) following treatment with different combinations of antibodies. Antibodies were the commercially available anti-TIM-3 antibody, F38-2E2; anti-TIM-3 antibody mAb15 generated as described in Example 1; R&D monoclonal anti-LILRB2 antibody; and anti-LILRB2 antibody, 42D1. mIgG1 served as an isotype negative control antibody.

FIGS. 9A-9I show graphs demonstrating release of IL-1β (FIGS. 9A, 9D and 9G), TNFα (FIGS. 9B, 9F and 9H) and IL-6 (FIGS. 9C, 9E and 9I) from macrophages stimulated by HMGB-1 (FIGS. 9A-9C) or CD40L (FIGS. 9D-9I) following treatment with antibodies. Antibodies were the commercially available anti-TIM-3 antibody, F38-2E2, and anti-TIM-3 antibody mAb15 generated as described in Example 1. mIgG1 served as an isotype negative control antibody.

FIG. 10 shows a graph demonstrating a dose curve of release of TNFα from macrophages stimulated by HMGB-1 following treatment with different doses of antibodies. Antibodies were the commercially available anti-TIM-3 antibody F38-2E2 (circles) and anti-TIM-3 antibody mAb15 (squares). Data were normalized.

FIG. 11A shows a graph demonstrating a dose curve of release of IL-1β from macrophages stimulated by LPS following treatment with different doses of antibodies. Antibodies were the commercially available anti-TIM-3 antibody F38-2E2 (circles), anti-TIM-3 antibody mAb15 (diamonds), and commercially available anti-LILRB2 antibody (clone 287219) (triangles). Data were collected at day 1-post-treatment. Data were normalized.

FIG. 11B shows a graph demonstrating a dose curve of release of TNFα from macrophages stimulated by LPS following treatment with different doses of antibodies. Antibodies were the commercially available anti-TIM-3 antibody F38-2E2 (circles), anti-TIM-3 antibody mAb15 (diamonds), and commercially available anti-LILRB2 antibody (triangles). Data were collected at day 3 post-treatment. Data were normalized.

FIGS. 12A-12D show graphs demonstrating dose curve of release of IL-1β (FIG. 12A), IL-6 (FIG. 12B), GM-CSF (FIG. 12C) and TNFα (FIG. 12D) from macrophages stimulated by LPS following treatment with different doses of antibodies. Antibodies were the commercially available anti-TIM-3 antibody F38-2E2 (circles), anti-TIM-3 antibody mAb15 (squares) and anti-LILRB2 (clone 287219) (inverted triangles). mIgG1 isotype (diamonds) and no antibody (triangles) served as a negative control. Data were collected at 24 hr, 48 hr, and 3 days post-treatment as indicated.

FIG. 13 shows graphs showing that PBMC from a donor with low LILRB2 showed diminished modulation of GM-CSF, IL-1β, and TNFα expression with mAb15 compared to F38-2E2. Donor KP42331 expressed LILRB2 (left panel, top). Donor KP42334 showed low expression of LILRB2 (left panel, bottom). Antibodies were the commercially available anti-TIM-3 antibody F38-2E2 and anti-TIM-3 antibody mAb15. mIgG1 served as an isotype control.

FIG. 14 shows graphs demonstrating expression of different LILRB proteins from donors KP42331 (normal levels of expression of LILRB2) and KP42334 (low levels of expression of LILRB2). Isotype represents a negative control. Expression of TIM-3 was determined using mAb15.

FIG. 15A shows graphs demonstrating expression of GM-CSF, IL-1α, IL-1β, IL-6 and TNFα from activated PBMCs from a donor with normal expression of LILRB2 (KP42331) and from a donor with low expression of LILRB2 (KP42334) following treatment with mAb15 or a mIgG1 isotype control.

FIG. 15B shows graphs demonstrating expression of IL-10, CCL2, CCL3, CCL4 and CCL5 from activated PBMCs from a donor with normal expression of LILRB2 (KP42331) and from a donor with low expression of LILRB2 (KP42334) following treatment with mAb15 or a mIgG1 isotype control.

FIG. 16 shows a sequence alignment of human TIM-3 (SEQ ID NO:99) and mouse TIM-3 (SEQ ID NO:100) including locations of the BC loop, the CC′ loop, the C′C″ loop, the DE loop, the FG loop and the mucin domain. The dots represent identities and the tildes represent insertions in the alignment.

FIGS. 17A-17F shows graphs demonstrating expression of GM-CSF (FIG. 17A), IL-6 (FIG. 17B), TNFα (FIG. 17C), IL-1β(FIG. 17D), IL-10 (FIG. 17E), and CCL5 (FIG. 17F) from activated macrophages from two different donors one day following treatment with anti-TIM-3 antibodies. Controls included F38-2E2, a commercially available anti-TIM-3 antibody and mIgG isotype controls. FIG. 17G shows the impact of anti-TIM-3 antibody mAb15 in the macrophage activation assay as examined at the transcriptional level.

FIG. 18 shows mixed lymphocyte reaction on day 1 or day 7 following treatment with F38-2E2, mAb15 or an isotype control. Supernatants were assessed for their expression of IL-1β, TNFα and IFN-γ at the time points indicated.

FIGS. 19A-19C show that ovarian cancer responds to anti-TIM-3 blockade in histoculture assay. Data are presented as fold change over isotype control and is representative of two independent experiments. Data are presented for human IL-1β (FIG. 19A), IL-8 (FIG. 19B), and IL-6 (FIG. 19C). All three cytokines increased in response to anti-TIM-3 compared to isotype control, with the greatest increase seen for IL-6 and IL-8 at 6 hours and for IL-1β at 24 hours post treatment.

### DETAILED DESCRIPTION OF THE INVENTION

Embodiments provided herein relate to antibodies that modulate (e.g., inhibit) the interaction of TIM-3 and LILRB2 and their use in various methods to determine and/or deliver appropriate cancer therapies and/or methods for increasing markers associated with M1 macrophages and/or methods for decreasing markers associated with M2 macrophages and/or methods for increasing production of cytokines and/or increasing cytokine secretion and/or methods for increasing T-cell proliferation. In some embodiments, the antibodies bind TIM-3 and inhibit the interaction of TIM-3 with LILRB2. In other embodiments, the antibodies bind LILRB2 and inhibit the interaction of LILRB2 with TIM-3. In some embodiments, the antibodies bind TIM-3 and increase markers associated with M1 macrophages and/or decrease markers associated with M2 macrophages. In some embodiments, the antibodies bind TIM-3 and increase production of cytokines and/or increase cytokine secretion. In some embodiments, the antibodies bind TIM-3 and increase T-cell proliferation.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X”.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer or TIM-3 related disorder). In some embodiments, the reference sample is from the same subject, but from a point in time before the subject developed cancer. In some embodiments, the reference sample is from a benign cancer sample (for example, benign breast cancer sample), from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted requirements to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“TIM-3” as used herein, refers to a type I transmembrane protein belonging to the TIM family, alternatively known as Hepatitis A virus cellular receptor 2 (HAVCR2), T cell immunoglobulin and mucin domain-containing protein-3 (TIMD-3), or Kidney Injury Molecule-3 (KIM-3). TIM-3 is expressed on, at least, T-helper 1 (Th1) cells, T-helper 17 (Th17) cells, IFN-γ producing CD8+ cytotoxic T 1 (Tc1) cells, as well as some dendritic cells (DC), macrophages, natural killer (NK) cells, natural killer T (NKT) cells and human monocytes. See Freeman et al., TIM genes: a family of cell surface phosphatidylserine receptors that regulate innate and adaptive immunity. (2010) Immunol. Rev. 235: 172-189.).

Human TIM-3 is believed to be 301 amino acids long with residues 1-21 encoding a signal peptide; residues 22-202 encoding the TIM-3 extracellular domain; residues 203-223 encoding a helical, transmembrane domain; and residues 224-301 encoding the cytoplasmic portion of TIM-3 (all residue numbers refer to SEQ ID NO:1). Within the extracellular domain, it is believed that residues 22-124 encode an Ig-like V-type (IgV) domain followed by the mucin domain (starting at about residue 125 and ending at about residue 182) and the stalk domain (starting at about residue 183 and ending at about residue 202) (all residue numbers refer to SEQ ID NO:1). Also within the extracellular domain, the cleft and/or FG loop domain (where residues 50, 62, 69, 112, and 121 are predicted to be involved in ligand binding) is predicted to start at about residue 49 and extend to about residue 122 (all residue numbers refer to SEQ ID NO:1). See 84868 (Entrez); ENSG00000135077 (Ensemble); Q8TDQ0 (UniProt); and NM_032782.4 (human RNA sequence) and NP_116171 (human polypeptide sequence) (NCBI); and Cao, E. et al. T cell immunoglobulin Mucin-3 crystal structure reveals a galactin-9-independent ligand-binding surface. Immunity (2007) 26:311-321, each of which is herein incorporated by reference in its entirety for all purposes.

The TIM-3 gene is believed to be located at chromosome 5 (156.51-156.57 Mb). Two isoforms or alternatively spliced forms of the human TIM-3 have been reported: Isoform 1 (UniProt:Q8TDQ0-1) and Isoform 2 (UniProt:Q8TDQ0-2). Several additional natural human TIM-3 variants have also been reported. In one variation of TIM-3 isoform 1, as an alternative sequence is found at residues 132-142. The residues AKVTPATTRQT (SEQ ID NO:101) in isoform 1 are replaced by residues GEWTGFACHLYE (SEQ ID NO:102) in isoform 2. Amino acids at residues 143-301 of isoform 1 are missing in isoform 2. A natural variant occurs at residue 140 of isoform 1 where a R to L substitution may occur (Monney, L. Nature (2002) 415:536-541). Accordingly, the present invention, in some aspects and embodiments, relates to therapeutic agents (e.g. antibodies, including bi-specific or multispecific antibodies and antibodies that competitively inhibit and/or bind the same epitope as a TIM-3 antibody disclosed herein) that bind to one, some or all of the human TIM-3 isoforms, alternatively spliced polypeptides and/or natural variants (e.g. including, without limitation, therapeutic agents (e.g. antibodies) that bind Isoform 1 or Isoform 2; or Isoforms 1 and 2) that may be specifically expressed in tumors or non-tumor cells.

Murine TIM-3 (NCBI Reference Sequence: NM_134250.2; SEQ ID NOs:9 and 10) is believed to be approximately 343 amino acids long with residues 1-22 encoding a signal peptide. See 102657 (Entrez); ENSMUSG00000020399 (Ensemble); Q6U7R4 (UniProt); and NM_134520 (murine RNA sequence) and NP_599011 (murine polypeptide sequence) (NCBI), each of which is herein incorporated by reference in its entirety for all purposes. The murine gene is believed to be located at chromosome 11 (46.45-46.48 Mb). TIM-3 is a highly conserved molecule, bearing 63% sequence homology between mice and humans.

“LILRB2” or “ILT-4” as used herein refers to “Leukocyte immunoglobulin-like receptor subfamily B member 2.” LILRB2 is also known as CD85 antigen-like family member D, CD85d, CD85D, ILT-4, Immunoglobulin-like transcript 4, Leukocyte immunoglobulin-like receptor 2, Leukocyte immunoglobulin-like receptor subfamily B member 2, LILRA6, LIR2, LIR-2, MIR10, MIR-10, and Monocyte/macrophage immunoglobulin-like receptor 10. LILRB2 is a protein that in humans is encoded by the LILRB2 gene. LILRB2 is a member of the leukocyte immunoglobulin-like receptor (LIR) family, and the gene encoding LILRB2 is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). ITIM motif 1 is found at residues 530-535. ITIM motif 2 is found at residues 559-564. ITIM motif 3 is found at residues 589-594. The receptor is expressed on immune cells where it binds to MHC class I molecules on antigen-presenting cells and transduces a negative signal that inhibits stimulation of an immune response. Multiple transcript variants encoding different isoforms have been found for this gene including variant 1 (GenBank Accession No. NP_005865; SEQ ID NO:5 and GenBank Accession No. NM_005874; SEQ ID NO:6) and variant 2 (GenBank Accession No. NP_001074447; SEQ ID NO:7 and GenBank Accession No. NM_001080978.3; SEQ ID NO:8). Variant 2 uses an alternate in-frame splice site in the central coding region, compared to variant 1.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a TIM-3 epitope is an antibody that binds this epitope with greater affinity, avidity, more readily, and/or with greater duration than it binds to other TIM-3 epitopes or non-TIM-3 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), for example, at least 90% pure, at least 95% pure, yet more preferably, at least 98% pure, and most preferably, at least 99% pure.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antibody specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antibody specific to the epitope binds. It is noted that, in some embodiments, not every one of the residues within the linear epitope need be directly bound (or involved in a bond) with the antibody. In some embodiments, linear epitopes can be from immunizations with a peptide that effectively consisted of the sequence of the linear epitope, or from structural sections of a protein that are relatively isolated from the remainder of the protein (such that the antibody can interact, at least primarily), just with that sequence section.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the IMGT definition, and/or the contact definition. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. The IMGT definition can include, for example, CDRs (CDR-L1, CDR-L2, CDR-L3, CDR-H1, CDR-H2, and CDR-H3) at amino acid residues 27-32 of L1, 50-52 of L2, 89-97 of L3, 26-35 of H1, 51-57 of H2, and 93-102 of H3 (as determined according to the methods described on the world wide web at www.imgt.org/IMGTScientificChart/ as of Jan. 4, 2016). CDRs can also be provided as shown in any one or more of the accompanying figures. With the exception of CDR1 in V_{H}, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hypervariable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an c constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LVR1, framework (FR) 2, LVR2, FR3, and LVR3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Nonlimiting exemplary light chain constant regions include λ and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{d}). Affinity can be measured by common methods known in the art (such as, for example, ELISA K_{D}, KinExA and/or surface plasmon resonance devices (such as a BIAcore® device), including those described herein.

The term “K_{D}”, as used herein, refers to the equilibrium dissociation constant of an antibody-antigen interaction.

In some embodiments, the “K_{D},” “K_{d},” “Kd” or “Kd value” of the antibody is measured by using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL/minute to achieve approximately 10 response units (RU) of coupled protein. Following the injection of antigen, 1 M ethanolamine is injected to block unreacted groups. For kinetics measurements, serial dilutions of polypeptide, for example, full length antibody, are injected in PBS with 0.05% TWEEN-20™ surfactant (PBST) at 25° C. at a flow rate of approximately 25 μL/min Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, for example, Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

In some embodiments, the difference between said two values (for example, K_{d} values) is substantially the same, for example, less than about 50%, less than about 40%, less than about 30%, less than about 20%, and/or less than about 10% as a function of the reference/comparator value.

In some embodiments, the difference between said two values (for example, K_{d} values) is substantially different, for example, greater than about 10%, greater than about 20%, greater than about 30%, greater than about 40%, and/or greater than about 50% as a function of the value for the reference/comparator molecule.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51:19-26.

The term “kₒₙ”, as used herein, refers to the rate constant for association of an antibody to an antigen. Specifically, the rate constants (kₘ and k_{off}) and equilibrium dissociation constants are measured using Fab antibody fragments (that is, univalent) and TIM-3. “Kₒₙ”, “kₒₙ”, “association rate constant”, or “kₐ”, are used interchangeably herein. The value indicates the binding rate of a binding protein to its target antigen or the rate of complex formation between an antibody and antigen, shown by the equation: Antibody(“Ab”)+Antigen(“Ag”)→Ab-Ag.

The term “k_{off}”, as used herein, refers to the rate constant for dissociation of an antibody from the antibody/antigen complex. k_{off} is also denoted as “K_{off}” or the “dissociation rate constant”. This value indicates the dissociation rate of an antibody from its target antigen or separation of Ab-Ag complex over time into free antibody and antigen as shown by the equation: Ab+Ag←Ab−Ag.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity.

The phrase “TIM-3 activity” indicates at least one of the biologically relevant functions of the TIM-3 protein. In some embodiments, this can be mediated by through the binding of the TIM-3 protein to a TIM-3 ligand.

The phrase “LILRB2 activity” indicates at least one of the biologically relevant functions of the LILRB2 protein. In some embodiments, this can be mediated by through the binding of the LILRB2 protein to a ligand of LILRB2; for example, HLA-G.

As used herein, the term “myeloid-associated cytokine” refers to cytokines produced by and/or that interact with cells of myeloid lineage; for example, cytokines produced by or that interact with monocytes and/or macrophages and/or dendritic cells. In some nonlimiting examples, a myeloid-associated cytokine that interacts with a macrophage and/or dendritic cell binds to or activates the macrophage or dendritic cells.

An “agonist” or “activating” antibody is one that increases and/or activates a biological activity of the protein e.g., a TIM-3 or LILRB2 protein. In some embodiments, the agonist antibody binds to an antigen and increases its biologically activity by at least about 20%, 40%, 60%, 80%, 85% or more.

An “antagonist”, a “blocking” or “neutralizing” antibody is one that decreases and/or inactivates a biological activity of the protein; e.g., a TIM-3 or LILRB2 protein. In some embodiments, the neutralizing antibody binds to an antigen and reduces its biological activity by at least about 20%, 40%, 60%, 80%, 85% 90%, 95%, 99% or more.

An “affinity matured” antibody refers to an antibody with one or more alterations in one or more CDRs compared to a parent antibody which does not possess such alterations, such alterations resulting in an improvement in the affinity of the antibody for antigen.

A “chimeric antibody” as used herein refers to an antibody in which a portion of the heavy and/or light chain is derived from a particular source or species, while at least a part of the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species. The chimeric construct can also be a functional fragment, as noted above.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

An “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

A “functional Fc region” possesses an “effector function” of a native sequence Fc region. Exemplary “effector functions” include Fc receptor binding; C1q binding; CDC; ADCC; phagocytosis; down regulation of cell surface receptors (for example B-cell receptor; BCR), etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immuno methods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation.

“Human effector cells” are leukocytes which express one or more FcRs and perform effector functions. In some embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T-cells, and neutrophils. The effector cells may be isolated from a native source, for example, from blood.

“Antibody-dependent T-cell-mediated cytotoxicity” and “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. No. 7,923,538, and U.S. Pat. No. 7,994,290.

“Complement dependent cytotoxicity” and “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, for example, as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, for example, in U.S. Pat. No. 6,194,551 B1, U.S. Pat. No. 7,923,538, U.S. Pat. No. 7,994,290 and WO 1999/51642. See also, for example, Idusogie et al., J. Immunol. 164: 4178-4184 (2000).

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, for example, 0-20% binding to the FcR compared to a native sequence IgG Fc region.

The polypeptide variant which “mediates antibody-dependent cell-mediated cytotoxicity (ADCC) in the presence of human effector cells more effectively” than a parent antibody is one which in vitro or in vivo is more effective at mediating ADCC, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

The term “leader sequence” refers to a sequence of amino acid residues located at the N-terminus of a polypeptide that facilitates secretion of a polypeptide from a mammalian cell. A leader sequence can be cleaved upon export of the polypeptide from the mammalian cell, forming a mature protein. Leader sequences can be natural or synthetic, and they can be heterologous or homologous to the protein to which they are attached.

A “native sequence” polypeptide comprises a polypeptide having the same amino acid sequence as a polypeptide found in nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (for example, an extracellular domain sequence), naturally occurring variant forms (for example, alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) as provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases. The terms “cancer” and “tumor” encompass solid and hematological/lymphatic cancers and also encompass malignant, pre-malignant, and benign growth, such as dysplasia. Also, included in this definition are cells having abnormal proliferation that is not impeded (e g immune evasion and immune escape mechanisms) by the immune system (e.g. virus infected cells). Exemplary tumor cells include, but are not limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The term “non-tumor cell” as used herein refers to a normal cells or tissue. Exemplary non-tumor cells include, but are not limited to: T-cells, B-cells, natural killer (NK) cells, natural killer T (NKT) cells, dendritic cells, monocytes, macrophages, epithelial cells, fibroblasts, hepatocytes, interstitial kidney cells, fibroblast-like synoviocytes, osteoblasts, and cells located in the breast, skeletal muscle, pancreas, stomach, ovary, small intestines, placenta, uterus, testis, kidney, lung, heart, brain, head and neck, liver, prostate, colon, lymphoid organs, bone, and bone-derived mesenchymal stem cells. The term “a cell or tissue located in the periphery” as used herein refers to non-tumor cells not located near tumor cells and/or within the tumor microenvironment.

The term “cells or tissue within the tumor microenvironment” as used herein refers to the cells, molecules, extracellular matrix and/or blood vessels that surround and/or feed a tumor cell. Exemplary cells or tissue within the tumor microenvironment include, but are not limited to: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells; macrophages; neutrophils; and other immune cells located proximal to a tumor. Methods for identifying tumor cells, and/or cells/tissues located within the tumor microenvironment are well known in the art, as described herein, below.

As used herein, the phrase “inhibiting or reducing T cell activation” refers to decreasing the activity of a target T cell subpopulation(s), as measured using a suitable in vitro, cellular, or in vivo assay. In particular, “reducing” or “inhibiting” can mean decreasing a (relevant or intended) biological activity of a target T cell subpopulation(s), as measured using a suitable in vitro, cellular or in vivo assay (which will usually depend on the target involved), by: at least about 5%, at least about 10%, at least about 25%, at least about 50%, at least about 60%, at least about 70%, at least about 80%, at least about 90%, or more, inclusive, compared to activity of the target in the same assay under the same conditions but without the presence of an agent. A “decrease” refers to a statistically significant decrease. For the avoidance of doubt, an decrease will be at least about 10% relative to a reference, such as at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, inclusive. As will be clear to the skilled person, “inhibiting” can also involve effecting a change in affinity, avidity, specificity and/or selectivity of a target or antigen, for one or more of its ligands, binding partners, partners for association into a homomultimeric or heteromultimeric form, or substrates; effecting a change and/or decrease in the sensitivity of the target or antigen for one or more conditions in the medium or surroundings in which the target or antigen is present (such as pH, ion strength, the presence of co-factors, etc.); and/or cellular proliferation or cytokine production compared to the same conditions but without the presence of an antibody, bispecific or multispecific polypeptide agent. This can be determined in any suitable manner and/or using any suitable assay known per se or described herein, depending on the target involved.

As used herein, the term “tolerance” or “tolerance to a tumor” refers to tumor-induced tolerance and/or immune suppression caused by the tumor. In particular immunological tolerance refers to a state of immune unresponsiveness specific to a particular tumor antigen or a set of tumor antigens. The phrase can refer to decreasing the activity of immune cell populations or subpopulations, as measured using a suitable in vitro, cellular, or in vivo assay to determine “change or modulation” of the activity and/or population of immune cells within the tumor and/or tumor microenvironment. In particular, “change or modulation” can mean increasing decreasing a (relevant or intended) biological activity of a target T-cell subpopulation(s), as measured using a suitable in vitro, cellular or in vivo assay (which will usually depend on the target involved), by at least about 5%, at least about 10%, at least about 20%, at least about 25%, at least about 50%, at least about 60%, at least about 70%, at least about 80%, at least about 90%, or more, inclusive, compared to activity of the target in the same assay under the same conditions but without the presence of an agent.

An “increase or decrease” refers to a statistically significant increase or decrease respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of an antibody, bispecific or multispecific polypeptide agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, cancer or cancer metastasis). “An immune response” can encompass aspects of both the innate and adaptive immune systems.

The term “cytokine” is a generic term for proteins released by one cell population which act on another cell as intercellular mediators. Examples of such cytokines are lymphokines, monokine secretions, and traditional polypeptide hormones. Included among the cytokines are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and tumor necrosis factor-β; mullerian-inhibiting substance; mouse gonadotropin-associated peptide; inhibin; activin; vascular endothelial growth factor; integrin; thrombopoietin (TPO); nerve growth factors such as NGF-α; platelet-growth factor; transforming growth factors (TGFs) such as TGF-α and TGF-β; insulin-like growth factor-I and -II; erythropoietin (EPO); osteo inductive factors; interferons such as, for example, interferon-α, interferon-β and interferon-γ (and interferon type I, II, and III), colony stimulating factors (CSFs) such as macrophage-CSF (M-CSF); granulocyte-macrophage-CSF (GM-CSF); and granulocyte-CSF (G-CSF); interleukins (ILs) such as, for example, IL-1, IL-1α, IL-2, IL-3, IL-4, IL-5, IL-6, IL-7, IL-8, IL-9, IL-10, IL-11, IL-12; a tumor necrosis factor such as, for example, TNFα or TNF-β; and other polypeptide factors including, for example, LIF and kit ligand (KL); chemokine (C-C motif) ligands (CCLs) such as CCL1, CCL2 CCL3, CCL4, and CCL5. As used herein, the term cytokine includes proteins obtained from natural sources or produced from recombinant bacterial, eukaryotic or mammalian cell culture systems and biologically active equivalents of the native sequence cytokines.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a TIM-3 antibody. “Ameliorating” also includes shortening or reduction in duration of a symptom.

The term “biological sample” means a quantity of a substance from a living thing or formerly living thing Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” refers to a composition known to not contain an analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (for example, analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (for example, severity of disease, progression/nonprogression/improvement, etc.). While the present disclosure may provide exemplary predetermined levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

The terms “inhibition” or “inhibit” refer to a decrease or cessation of any phenotypic characteristic or to the decrease or cessation in the incidence, degree, or likelihood of that characteristic; for example the interaction of TIM-3 and LILRB2. To “reduce” or “inhibit” is to decrease, reduce or arrest an activity, function, and/or amount as compared to a reference. In some embodiments, by “reduce” or “inhibit” is meant the ability to cause an overall decrease of 1% or greater. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time.

As used herein, to “stimulate” a function or activity is to increase the function or activity when compared to otherwise same conditions except for a condition or parameter of interest, or alternatively, as compared to another condition. For example, an antibody which stimulates cytokine secretion results in increased secretion of the cytokine compared to the rate of secretion of cytokine in the absence of the antibody.

As used herein, to “suppress” a function or activity is to decrease or reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody. In another example, an antibody which suppresses cytokine secretion results in decreased secretion of the cytokine compared to the rate of secretion of cytokine in the absence of the antibody.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Methods and Compositions Relating to TIM-3

The interaction between cancer and the immune system is complex and multifaceted. See de Visser et al., Nat. Rev. Cancer (2006) 6:24-37. While many cancer patients appear to develop an anti-tumor immune response, cancers also develop strategies to evade immune detection and destruction. Recently, immunotherapy has been developed for the treatment and prevention of cancer and other disorders Immunotherapy provides the advantage of cell specificity that other treatment modalities lack. As such, methods for enhancing the efficacy of immune based therapies can be clinically beneficial.

The therapeutic agents (e.g. antibodies) modulate the interaction of TIM-3 and LILRB2. In some embodiments, the antibody binds TIM-3. In other embodiments, the antibody binds LILRB2. In some embodiments, the modulation of the interaction is an inhibition of the interaction of TIM-3 and LILRB2; for example, inhibition of the binding of TIM-3 and LILRB2. Blocking the interaction of TIM-3 and LILRB2 leads to the secretion of myeloid-associated pro-inflammatory cytokines; for example, cytokines produced by or that interact with macrophages. In some embodiments, the antibodies block the interaction of TIM-3 and LILRB2 such that macrophages are preferentially activated. In some embodiments, the antibodies block the interaction of TIM-3 and LILRB2 such that dendritic cells are preferentially activated. In some embodiments, the antibodies block the interaction of TIM-3 and LILRB2 such that macrophages and dendritic cells are preferentially activated. In some embodiments, the antibody is not antibody F38-2E2 or a functional equivalent of antibody F38-2E2 with respect to the inhibition of the interaction of TIM-3 and LILRB2 by antibody F38-2E2. F38-2E2 is a mouse IgG1 antibody with a κ light chain that has specificity for human TIM3 protein. It is available for purchase from Biolegend (San Diego, Calif., USA) in ULTRA-LEAF quality (low endotoxin, azide-free), catalogue number 345010.

In some embodiments, the antibodies of the invention inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion (See, e.g., Virgin et al. (2009) Cell 138:30-50). Accordingly, as used herein the term “a pro-tumor T cell” refers to a state of T cell dysfunction that arises during many chronic infections and cancer. This dysfunction is defined by poor proliferative and/or effector functions, sustained expression of inhibitory receptors and a transcriptional state distinct from that of functional effector or memory T cells. Exhaustion prevents optimal control of infection and tumors. See Wherry, J. W. T cell exhaustion. Nat Immunol (2011) 12:492-499. In addition, as used herein, the term “an anti-tumor CD8+ and/or CD4+ T cell” refers to T cells that can mount an immune response to a tumor. Exemplary pro-tumor T cells include, but are not limited to, Tregs, CD4+ and/or CD8+ T cells expressing one or more checkpoint inhibitory receptors, Th2 cells and Th17 cells. The term “checkpoint inhibitory receptors”, as used herein, refers to receptors (e.g. CTLA-4, B7-H3, B7-H4, PD-1, TIM-3) expressed on immune cells that prevent or inhibit uncontrolled immune responses. See Stagg, J. et al., Immunotherapeutic approach in triple-negative breast cancer. Ther Adv Med Oncol. (2013) 5(3):169-181. Thus, in some embodiments, inhibition of TIM-3 activity can include reducing the level of and/or preventing the inhibition of T cell proliferation. In some embodiments, this can also be described as restoring and/or increasing T cell proliferation. In some embodiments, the inhibition of TIM-3 activity can also be described as restoring and/or increasing myeloid cell proliferation, activation and/or differentiation; for example, activation of monocytes, macrophages, and/or dendritic cells.

In some embodiments, the modulation of the interaction is an inhibition of the interaction of TIM-3 and LILRB2; for example, inhibition of the binding of TIM-3 and LILRB2. Blocking the interaction of TIM-3 and LILRB2 leads to the secretion of myeloid-associated pro-inflammatory cytokines; for example, cytokines produced by or that interact with macrophages. In some embodiments, the antibodies block the interaction of TIM-3 and LILRB2 such that macrophages are preferentially activated. In some embodiments, the antibodies block the interaction of TIM-3 and LILRB2 such that dendritic cells are preferentially activated. In some embodiments, the antibodies block the interaction of TIM-3 and LILRB2 such that macrophages and dendritic cells are preferentially activated. In some embodiments, the antibody is not antibody F38-2E2 or a functional equivalent of antibody F38-2E2 with respect to the inhibition of the interaction of TIM-3 and LILRB2 by antibody F38-2E2.

Despite recent advances, a need has been identified for more effective treatments of cancer utilizing immunotherapy. More particularly, a need has been identified for novel anti-TIM-3 antibodies or antibodies that inhibit the interaction of TIM-3 its ligands and methods that modulate TIM-3 activity which are capable of enhancing the host immune response against tumors for treating cancer. For example, to allow for increased T-cell proliferation, for example, for the treatment of cancer.

### Methods of Treating Diseases Using TIM-3 Antibodies

In some aspects, the invention provides methods of stimulating the secretion of a myeloid-associated cytokine in an individual. The method comprises administering to the individual, an effective amount of an antibody that modulates the interaction of TIM-3 and LILRB2. In some embodiments, the modulation of the interaction of TIM-3 and LILRB2 is an inhibition of the interaction of TIM-3 and LILRB2; for example, by inhibiting the binding of TIM-3 and LILRB2. The inhibition of the interaction of TIM-3 and LILRB2 may lead to the activation of monocytes; e.g., macrophages, which leads to the secretion of pro-inflammatory cytokines. In some embodiments, the antibody binds TIM-3. In other embodiments, the antibody binds LILRB2. In some embodiments, binding of the antibody to TIM-3 or LILRB2 leads to the preferential activation of macrophages and/or the preferential secretion of pro-inflammatory myeloid-associated cytokines. In some embodiments, binding of the antibody to TIM-3 or LILRB2 leads to the preferential activation of dendritic cells and/or the preferential secretion of pro-inflammatory myeloid-associated cytokines. In some embodiments, binding of the antibody to TIM-3 or LILRB2 leads to the preferential activation of macrophages and dendritic cells and/or the preferential secretion of pro-inflammatory myeloid-associated cytokines. In some embodiments, the individual is human.

In some embodiments, the pro-inflammatory cytokine is IL-12, TNFα, IL-1β, GM-CSF, or IL-6. In some embodiments, any one, any two, any three, any four, or all five cytokines are secreted by monocytes or macrophages following administration of an antibody that inhibits the interaction of TIM-3 and LILRB2. In some embodiments, one or more of pro-inflammatory cytokine is IL-12, TNFα, IL-1β, GM-CSF, or IL-6 is secreted by or interacts with monocytes following administration of an antibody that inhibits the interaction of TIM-3 and LILRB2. In some embodiments, secretion of pro-inflammatory cytokines following administration of an antibody of the invention is increased compared to secretion of pro-inflammatory cytokines following administration of antibody F38-2E2. In some embodiments, the secretion of pro-inflammatory cytokines (e.g., IL-12, TNFα, IL-1β, GM-CSF, or IL-6) is at least about any of 2-fold, 3-fold, 4-fold, 5-fold, 6-fold, 7-fold, 8-fold, 9-fold, or 10-fold following administration of an antibody of the invention compared to secretion of pro-inflammatory cytokines following administration of antibody F38-2E2. In some embodiments, activation of macrophages is increased by at least about any of 2-fold, 3-fold, 4-fold, 5-fold, 6-fold, 7-fold, 8-fold, 9-fold, or 10-fold following administration of an antibody of the invention compared to activation of macrophages following administration of antibody F38-2E2. In some embodiments, activation of dendritic cells is increased by at least about any of 2-fold, 3-fold, 4-fold, 5-fold, 6-fold, 7-fold, 8-fold, 9-fold, or 10-fold following administration of an antibody of the invention compared to activation of dendritic cells following administration of antibody F38-2E2. In some embodiments, activation of macrophages and dendritic cells is increased following administration of an antibody of the invention compared to activation of macrophages and dendritic cells following administration of antibody F38-2E2.

In some embodiments, treatment with the anti-TIM-3 and/or anti-LILRB2 antibody suppresses secretion of cytokines. In some embodiments, treatment with the anti-TIM-3 and/or anti-LILRB2 antibody of the invention suppresses expression of one or more of IL-10, CCL2, CCL3, CCL4 or CCL5. In some embodiments, treatment with the anti-TIM-3 and/or anti-LILRB2 antibody of the invention reduces expression of one or more of IL-10, CCL2, CCL3, CCL4 or CCL5. In some embodiments, secretion of any one, any two, any three, any four, or all five cytokines by monocytes or macrophages is suppressed following administration of an antibody that inhibits the interaction of TIM-3 and LILRB2. In some embodiments, secretion of cytokines following administration of an antibody of the invention is suppressed compared to suppression of cytokines following administration of antibody F38-2E2. In some embodiments, the secretion cytokines (e.g., IL-10, CCL2, CCL3, CCL4 or CCL5) is at least about any of 2-fold, 3-fold, 4-fold, 5-fold, 6-fold, 7-fold, 8-fold, 9-fold, or 10-fold suppressed following administration of an antibody of the invention compared to suppression of secretion cytokines following administration of antibody F38-2E2.

In some embodiments, the invention provides methods of stimulating the secretion of a myeloid-associated cytokine in an individual with cancer, wherein the method comprises administering to the individual, an effective amount of an antibody that modulates the interaction of TIM-3 and LILRB2. In some embodiments, the cytokines are secreted in a tumor; for example, pro-inflammatory cytokines are secreted by a monocyte, a macrophage or a dendritic cell located in or near a tumor. In some embodiments, the individual is human.

In some aspects, the invention provides methods for treating cancer in an individual. The method comprises administering to the individual, an effective amount of an antibody that modulates the interaction of TIM-3 and LILRB2. In some embodiments, the modulation of the interaction of TIM-3 and LILRB2 is an inhibition of the interaction of TIM-3 and LILRB2; for example, by inhibiting the binding of TIM-3 and LILRB2. The inhibition of the interaction of TIM-3 and LILRB2 may lead to the activation of monocytes; e.g., macrophages, which leads to the secretion of pro-inflammatory cytokines. In some embodiments, the antibody binds TIM-3. In other embodiments, the antibody binds LILRB2. In some embodiments, binding of the antibody to TIM-3 or LILRB2 leads to the preferential activation of macrophages and/or the preferential secretion of pro-inflammatory cytokines by macrophages. In other embodiments, the antibody binds LILRB2. In some embodiments, binding of the antibody to TIM-3 or LILRB2 leads to the preferential activation of dendritic cells and/or the preferential secretion of pro-inflammatory cytokines by dendritic cells. In some embodiments, binding of the antibody to TIM-3 or LILRB2 leads to the preferential activation of macrophages and dendritic cells and/or the preferential secretion of pro-inflammatory cytokines by macrophages and dendritic cells. In some embodiments, binding of the antibody to TIM-3 or LILRB2 leads to the preferential secretion of pro-inflammatory cytokines that interact with macrophages. In some embodiments, binding of the antibody to TIM-3 or LILRB2 leads to the preferential secretion of pro-inflammatory cytokines that interact with dendritic cells. In some embodiments, binding of the antibody to TIM-3 or LILRB2 leads to the preferential secretion of pro-inflammatory cytokines that interact with macrophages and dendritic cells. In some embodiments, the cytokines are secreted in a tumor; for example, pro-inflammatory cytokines are secreted by a monocyte, a macrophage or a dendritic cell located in or near a tumor. In some embodiments, the individual is human.

In some aspects, the invention provides methods of stimulating the secretion of a myeloid-associated cytokine in an individual comprising administering to the individual a therapeutically effective amount of antibody mAb15 or a functional equivalent thereof. In some embodiments, the invention provides methods to treat cancer in an individual comprising administering to the individual a therapeutically effective amount of antibody mAb15 or a functional equivalent thereof. In some embodiments, the antibody is a humanized mAb15. In some embodiments, the antibody binds the same epitope as antibody mAb15. In some embodiments, the invention provides antibodies that compete with antibody mAb15. In some embodiments, the antibody competes with mAb15 such that less than about any one of 90%, 80%, 70%, 60%, 50%, 40%, 30%, 20%, 10%, or 1% of mAb15 binds TIM-3 in the presence of the antibody of the invention.

In some aspects, the invention provides methods of stimulating the secretion of a myeloid-associated cytokine in an individual comprising administering to the individual a therapeutically effective amount of antibody mAb13 or a functional equivalent thereof. In some embodiments, the invention provides methods to treat cancer in an individual comprising administering to the individual a therapeutically effective amount of antibody mAb13 or a functional equivalent thereof. In some embodiments, the antibody is a humanized mAb13. In some embodiments, the antibody binds the same epitope as antibody mAb13. In some embodiments, the invention provides antibodies that compete with antibody mAb13. In some embodiments, the antibody competes with mAb13 such that less than about any one of 90%, 80%, 70%, 60%, 50%, 40%, 30%, 20%, 10%, or 1% of mAb13 binds TIM-3 in the presence of the antibody of the invention. In some embodiments, mAb13 has a light chain variable region comprising the amino acid sequence of SEQ ID NO:22 and a heavy chain variable region comprising the amino acid sequence of SEQ ID NO:21. In some embodiments, the antibody competes with mAb13 for binding TIM-3 and stimulates expression of one or more of IL-1β, TNF-α, IL-12, GM-CSF and/or IL-6 (e.g., by tumor macrophages). In some embodiments, the antibody competes with mAb13 for binding TIM-3 and increases the expression of one or more of IL-1β, TNF-α, IL-12, GM-CSF and/or IL-6 (e.g., by tumor macrophages). In some embodiments, the antibody competes for binding TIM-3 and suppresses secretion of one or more of IL-10, CCL2, CCL3, CCL4 and/or CCL5. In some embodiments, the antibody competes for binding TIM-3 and reduces secretion of one or more of IL-10, CCL2, CCL3, CCL4 and/or CCL5.

In some aspects, the invention provides methods of stimulating the secretion of a myeloid-associated cytokine in an individual comprising administering to the individual a therapeutically effective amount of antibody mAb17 or a functional equivalent thereof. In some embodiments, the invention provides methods to treat cancer in an individual comprising administering to the individual a therapeutically effective amount of antibody mAb17 or a functional equivalent thereof. In some embodiments, the antibody is a humanized mAb17. In some embodiments, the antibody binds the same epitope as antibody mAb17. In some embodiments, the invention provides antibodies that compete with antibody mAb17. In some embodiments, the antibody competes with mAb17 such that less than about any one of 90%, 80%, 70%, 60%, 50%, 40%, 30%, 20%, 10%, or 1% of mAb17 binds TIM-3 in the presence of the antibody of the invention. In some embodiments, mAb17 has a light chain variable region comprising the amino acid sequence of SEQ ID NO:24 and a heavy chain variable region comprising the amino acid sequence of SEQ ID NO:23. In some embodiments, the antibody competes with mAb17 for binding TIM-3 and stimulates expression of one or more of IL-1β, TNF-α, IL-12, GM-CSF and/or IL-6 (e.g., by tumor macrophages). In some embodiments, the antibody competes with mAb17 for binding TIM-3 and increases the expression of one or more of IL-1β, TNF-α, IL-12, GM-CSF and/or IL-6 (e.g., by tumor macrophages). In some embodiments, the antibody competes for binding TIM-3 and suppresses secretion of one or more of IL-10, CCL2, CCL3, CCL4 and/or CCL5. In some embodiments, the antibody competes for binding TIM-3 and reduces secretion of one or more of IL-10, CCL2, CCL3, CCL4 and/or CCL5.

In some aspects, the invention provides methods of stimulating the secretion of a myeloid-associated cytokine in an individual comprising administering to the individual a therapeutically effective amount of antibody mAb22 or a functional equivalent thereof. In some embodiments, the invention provides methods to treat cancer in an individual comprising administering to the individual a therapeutically effective amount of antibody mAb22 or a functional equivalent thereof. In some embodiments, the antibody is a humanized mAb22. In some embodiments, the antibody binds the same epitope as antibody mAb22. In some embodiments, the invention provides antibodies that compete with antibody mAb22. In some embodiments, the antibody competes with mAb22 such that less than about any one of 90%, 80%, 70%, 60%, 50%, 40%, 30%, 20%, 10%, or 1% of mAb22 binds TIM-3 in the presence of the antibody of the invention. In some embodiments, mAb22 has a light chain variable region comprising the amino acid sequence of SEQ ID NO:26 and a heavy chain variable region comprising the amino acid sequence of SEQ ID NO:25. In some embodiments, the antibody competes with mAb22 for binding TIM-3 and stimulates expression of one or more of IL-1β, TNF-α, IL-12, GM-CSF and/or IL-6 (e.g., by tumor macrophages). In some embodiments, the antibody competes with mAb22 for binding TIM-3 and increases expression of one or more of IL-1β, TNF-α, IL-12, GM-CSF and/or IL-6 (e.g., by tumor macrophages). In some embodiments, the antibody competes for binding TIM-3 and suppresses secretion of one or more of IL-10, CCL2, CCL3, CCL4 and/or CCL5. In some embodiments, the antibody competes for binding TIM-3 and reduces secretion of one or more of IL-10, CCL2, CCL3, CCL4 and/or CCL5.

In some embodiments, the invention provides methods of stimulating the secretion of a myeloid-associated cytokine in an individual comprising administering to the individual a therapeutically effective amount of antibody that binds TIM-3 comprising the three CDRs of the heavy chain of mAb15 set forth in SEQ ID NO:12 and the three CDRs of the light chain of mAb15 set forth in SEQ ID NO:14. In some embodiments, the antibody comprises three heavy chain CDRs comprising the amino acid sequences GYGVT (SEQ ID NO:59), MIWGDGNTDYNSGLKS (SEQ ID NO:80) and SYYYGPPDY (SEQ ID NO:81). In some embodiments, the antibody comprises three light chain CDRs comprising the amino acid sequences KSSQSLLNSRSQKNYLA (SEQ ID NO:88), FASTRES (SEQ ID NO:89) and HQHYNTPYT (SEQ ID NO:20). In some embodiments, the antibody comprises three heavy chain CDRs comprising the amino acid sequences GFSLTGYG (SEQ ID NO:15), IWGDGNT (SEQ ID NO:16) and ARSYYYGPPDY (SEQ ID NO:17). In some embodiments, the antibody comprises three light chain CDRs comprising the amino acid sequences QSLLNSRSQKNY (SEQ ID NO:18), FAS (SEQ ID NO:19) and HQHYNTPYT (SEQ ID NO:20). In some embodiments, the antibody of the invention competes with an antibody comprising the three CDRs of the heavy chain of mAb15 set forth in SEQ ID NO:12 and the three CDRs of the light chain of mAb15 set forth in SEQ ID NO:14. In some embodiments, the antibody competes with an antibody comprising the three CDRs of the heavy chain of mAb15 set forth in SEQ ID NO:12 and the three CDRs of the light chain of mAb15 set forth in SEQ ID NO:14 such that less than about any one of 90%, 80%, 70%, 60%, 50%, 40%, 30%, 20%, 10%, or 1% of an antibody comprising the three CDRs of the heavy chain of mAb15 set forth in SEQ ID NO:12 and the three CDRs of the light chain of mAb15 set forth in SEQ ID NO:14 binds TIM-3 in the presence of the antibody of the invention.

In some embodiments, the invention provides methods of stimulating the secretion of a myeloid-associated cytokine in an individual comprising administering to the individual a therapeutically effective amount of antibody that binds TIM-3 comprising the three CDRs of the heavy chain of mAb13 set forth in SEQ ID NO:21 and the three CDRs of the light chain of mAb13 set forth in SEQ ID NO:22. In some embodiments, the antibody comprises three heavy chain CDRs comprising the amino acid sequences DYYIN (SEQ ID NO:90), WIYPGSGNTKYNEKFKG (SEQ ID NO:28), and GGKYYAMDY (SEQ ID NO:29) and three light chain CDRs comprising the amino acid sequences KASQSVGNNVA (SEQ ID NO:30), YASNRYT (SEQ ID NO:31), and QQDYSSPYT (SEQ ID NO:32). In some embodiments the antibody comprises the three heavy chain CDRs comprising the amino acid sequences GYTFTDYYIN (SEQ ID NO:27), WIYPGSGNTKYNEKFKG (SEQ ID NO:28), and GGKYYAMDY (SEQ ID NO:29) and three light chain CDRs comprising the amino acid sequences KASQSVGNNVA (SEQ ID NO:30), YASNRYT (SEQ ID NO:31), and QQDYSSPYT (SEQ ID NO:32). In some embodiments the antibody comprises the three heavy chain CDRs comprising the amino acid sequences GYTFTDYYIN (SEQ ID NO:27), WIYPGSGNTKYNEKFKG (SEQ ID NO:28), and GGKYYAMDY (SEQ ID NO:29) and three light chain CDRs comprising the amino acid sequences KASQSVGNNVA (SEQ ID NO:30), YASNRYT (SEQ ID NO:31), and QQDYSSPYT (SEQ ID NO:32). In some embodiments, the antibody of the invention competes with an antibody comprising the three CDRs of the heavy chain of mAb13 set forth in SEQ ID NO:21 and the three CDRs of the light chain of mAb13 set forth in SEQ ID NO:22. In some embodiments, the antibody competes with an antibody comprising the three CDRs of the heavy chain of mAb13 set forth in SEQ ID NO:21 and the three CDRs of the light chain of mAb13 set forth in SEQ ID NO:22 such that less than about any one of 90%, 80%, 70%, 60%, 50%, 40%, 30%, 20%, 10%, or 1% of an antibody comprising the three CDRs of the heavy chain of mAb13 set forth in SEQ ID NO:21 and the three CDRs of the light chain of mAb13 set forth in SEQ ID NO:22 binds TIM-3 in the presence of the antibody of the invention. In some embodiments, the antibody that competes with mAb13 (or an antibody comprising the six CDRs of mAb13) stimulates the secretion of one or more of IL-1β, TNFα, IL-6, GM-CSF or IL-12. In some embodiments, the antibody that competes with mAb13 (or an antibody comprising the six CDRs of mAb13) increases the secretion of one or more of IL-1β, TNFα, IL-6, GM-CSF or IL-12. In some embodiments, the antibody that competes with mAb13 (or an antibody comprising the six CDRs of mAb13) suppresses secretion of one or more of IL-10, CCL2, CCL3, CCL4 or CCL5. In some embodiments, the antibody that competes with mAb13 (or an antibody comprising the six CDRs of mAb13) reduces secretion of one or more of IL-10, CCL2, CCL3, CCL4 or CCL5.

In some embodiments, the invention provides methods of stimulating the secretion of one or more myeloid-associated cytokines in an individual comprising administering to the individual a therapeutically effective amount of antibody that binds TIM-3 comprising the three CDRs of the heavy chain of mAb17 set forth in SEQ ID NO:23 and the three CDRs of the light chain of mAb17 set forth in SEQ ID NO:24. In some embodiments, the antibody comprises three heavy chain CDRs comprising the amino acid sequences NYGMS (SEQ ID NO:91), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QNSHSFPPT (SEQ ID NO:38). In some embodiments, the antibody comprises three heavy chain CDRs comprising the amino acid sequences GFTFSNYGMS (SEQ ID NO:33), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QNSHSFPPT (SEQ ID NO:38). In some embodiments, the antibody of the invention competes with an antibody comprising the three CDRs of the heavy chain of mAb17 set forth in SEQ ID NO:23 and the three CDRs of the light chain of mAb17 set forth in SEQ ID NO:24. In some embodiments, the antibody competes with an antibody comprising the three CDRs of the heavy chain of mAb17 set forth in SEQ ID NO:23 and the three CDRs of the light chain of mAb17 set forth in SEQ ID NO:24 such that less than about any one of 90%, 80%, 70%, 60%, 50%, 40%, 30%, 20%, 10%, or 1% of an antibody comprising the three CDRs of the heavy chain of mAb17 set forth in SEQ ID NO:23 and the three CDRs of the light chain of mAb17 set forth in SEQ ID NO:24 binds TIM-3 in the presence of the antibody of the invention. In some embodiments, the antibody that competes with mAb17 (or an antibody comprising the six CDRs of mAb17) stimulates the secretion of one or more of IL-1β, TNFα, IL-6, GM-CSF or IL-12. In some embodiments, the antibody that competes with mAb17 (or an antibody comprising the six CDRs of mAb17) increases the secretion of one or more of IL-1β, TNFα, IL-6, GM-CSF or IL-12. In some embodiments, the antibody that competes with mAb17 (or an antibody comprising the six CDRs of mAb17) suppresses secretion of one or more of IL-10, CCL2, CCL3, CCL4 or CCL5. In some embodiments, the antibody that competes with mAb17 (or an antibody comprising the six CDRs of mAb17) reduces secretion of one or more of IL-10, CCL2, CCL3, CCL4 or CCL5.

In some embodiments, the invention provides methods of stimulating the secretion of one or more myeloid-associated cytokines in an individual comprising administering to the individual a therapeutically effective amount of antibody that binds TIM-3 comprising the three CDRs of the heavy chain of mAb22 set forth in SEQ ID NO:25 and the three CDRs of the light chain of mAb22 set forth in SEQ ID NO:26. In some embodiments, the antibody comprises three heavy chain CDRs comprising the amino acid sequences NHGMS (SEQ ID NO:97), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QHSHSFPPT (SEQ ID NO:40). In some embodiments, the antibody comprises three heavy chain CDRs comprising the amino acid sequences GFTFSNHGMS (SEQ ID NO:39), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QHSHSFPPT (SEQ ID NO:40). In some embodiments, the antibody of the invention competes with an antibody comprising the three CDRs of the heavy chain of mAb22 set forth in SEQ ID NO:25 and the three CDRs of the light chain of mAb22 set forth in SEQ ID NO:26. In some embodiments, the antibody competes with an antibody comprising the three CDRs of the heavy chain of mAb22 set forth in SEQ ID NO:25 and the three CDRs of the light chain of mAb22 set forth in SEQ ID NO:26 such that less than about any one of 90%, 80%, 70%, 60%, 50%, 40%, 30%, 20%, 10%, or 1% of an antibody comprising the three CDRs of the heavy chain of mAb22 set forth in SEQ ID NO:25 and the three CDRs of the light chain of mAb22 set forth in SEQ ID NO:26 binds TIM-3 in the presence of the antibody of the invention. In some embodiments, the antibody that competes with mAb22 (or an antibody comprising the six CDRs of mAb22) stimulates the secretion of one or more of IL-1β, TNFα, IL-6, GM-CSF or IL-12. In some embodiments, the antibody that competes with mAb22 (or an antibody comprising the six CDRs of mAb22) increases the secretion of one or more of IL-1β, TNFα, IL-6, GM-CSF or IL-12. In some embodiments, the antibody that competes with mAb22 (or an antibody comprising the six CDRs of mAb22) suppresses secretion of one or more of IL-10, CCL2, CCL3, CCL4 or CCL5. In some embodiments, the antibody that competes with mAb22 (or an antibody comprising the six CDRs of mAb22) reduces secretion of one or more of IL-10, CCL2, CCL3, CCL4 or CCL5.

In some embodiments, the invention provides methods of stimulating the secretion of one or more myeloid-associated cytokines in an individual comprising administering to the individual a therapeutically effective amount of antibody that binds TIM-3 comprising the three CDRs of the heavy chain of mAb58 set forth in SEQ ID NO:53 and the three CDRs of the light chain of mAb58 set forth in SEQ ID NO:54. In some embodiments, the antibody comprises three heavy chain CDRs comprising the amino acid sequences TYGMS (SEQ ID NO:55), WINTYSGAPTYADDFKG (SEQ ID NO:56) and KPPHYYVNSFDY (SEQ ID NO:57) and three light chain CDRs comprising the amino acid sequences RASQSISDYLH (SEQ ID NO:58), YASQSIS (SEQ ID NO:37), and QNGHSFPYT (SEQ ID NO:60). In some embodiments, the antibody of the invention competes with an antibody comprising the three CDRs of the heavy chain of mAb58 set forth in SEQ ID NO:53 and the three CDRs of the light chain of mAb58 set forth in SEQ ID NO:54. In some embodiments, the antibody competes with an antibody comprising the three CDRs of the heavy chain of mAb58 set forth in SEQ ID NO:53 and the three CDRs of the light chain of mAb58 set forth in SEQ ID NOs:54 such that less than about any one of 90%, 80%, 70%, 60%, 50%, 40%, 30%, 20%, 10%, or 1% of an antibody comprising the three CDRs of the heavy chain of mAb58 set forth in SEQ ID NO:53 and the three CDRs of the light chain of mAb58 set forth in SEQ ID NO:54 binds TIM-3 in the presence of the antibody of the invention. In some embodiments, the antibody that competes with mAb58 (or an antibody comprising the six CDRs of mAb58) stimulates the secretion of one or more of IL-1β, TNFα, IL-6, GM-CSF or IL-12. In some embodiments, the antibody that competes with mAb58 (or an antibody comprising the six CDRs of mAb58) increases the secretion of one or more of IL-1β, TNFα, IL-6, GM-CSF or IL-12. In some embodiments, the antibody that competes with mAb58 (or an antibody comprising the six CDRs of mAb58) suppresses secretion of one or more of IL-10, CCL2, CCL3, CCL4 or CCL5. In some embodiments, the antibody that competes with mAb58 (or an antibody comprising the six CDRs of mAb58) reduces secretion of one or more of IL-10, CCL2, CCL3, CCL4 or CCL5.

In some embodiments, the invention provides methods of stimulating the secretion of one or more myeloid-associated cytokines in an individual comprising administering to the individual a therapeutically effective amount of antibody that binds TIM-3 comprising the three CDRs of the heavy chain of mAb48 set forth in SEQ ID NO:70 and the three CDRs of the light chain of mAb48 set forth in SEQ ID NO:71. In some embodiments, the antibody comprises three heavy chain CDRs comprising the amino acid sequences DYYIN (SEQ ID NO:90), WIYPGSGNTKYNEKFKG (SEQ ID NO:28) and DRFDY (SEQ ID NO:92) and three light chain CDRs comprising the amino acid sequences SASSGVSSSYLY (SEQ ID NO:93), STSNLAS (SEQ ID NO:94), and HQWSNSPYT (SEQ ID NO:95). In some embodiments, the antibody of the invention competes with an antibody comprising the three CDRs of the heavy chain of mAb48 set forth in SEQ ID NO:70 and the three CDRs of the light chain of mAb48 set forth in SEQ ID NO:71. In some embodiments, the antibody competes with an antibody comprising the three CDRs of the heavy chain of mAb48 set forth in SEQ ID NO:70 and the three CDRs of the light chain of mAb48 set forth in SEQ ID NOs:71 such that less than about any one of 90%, 80%, 70%, 60%, 50%, 40%, 30%, 20%, 10%, or 1% of an antibody comprising the three CDRs of the heavy chain of mAb48 set forth in SEQ ID NO:70 and the three CDRs of the light chain of mAb48 set forth in SEQ ID NO:71 binds TIM-3 in the presence of the antibody of the invention. In some embodiments, the antibody that competes with mAb48 (or an antibody comprising the six CDRs of mAb48) stimulates the secretion of one or more of IL-1β, TNFα, IL-6, GM-CSF or IL-12. In some embodiments, the antibody that competes with mAb48 (or an antibody comprising the six CDRs of mAb48) increases the secretion of one or more of IL-1β, TNFα, IL-6, GM-CSF or IL-12. In some embodiments, the antibody that competes with mAb48 (or an antibody comprising the six CDRs of mAb48) suppresses secretion of one or more of IL-10, CCL2, CCL3, CCL4 or CCL5. In some embodiments, the antibody that competes with mAb48 (or an antibody comprising the six CDRs of mAb48) reduces secretion of one or more of IL-10, CCL2, CCL3, CCL4 or CCL5.

In some embodiments, the invention provides methods of stimulating the secretion of one or more myeloid-associated cytokines in an individual comprising administering to the individual a therapeutically effective amount of antibody that binds TIM-3 comprising the three CDRs of the heavy chain of mAb15 set forth in SEQ ID NO:13 and the three CDRs of the light chain of mAb15 set forth in SEQ ID NO:14. In some embodiments, the antibody comprises three heavy chain CDRs comprising the amino acid sequences GYGVT (SEQ ID NO:59), MIWGDGNTDYNSGLKS (SEQ ID NO:80) and SYYYGPPDY (SEQ ID NO:81) and three light chain CDRs comprising the amino acid sequences KSSQSLLNSRSQKNYLA (SEQ ID NO:88), FASTRES (SEQ ID NO:89), and HQHYNTPYT (SEQ ID NO:20). In some embodiments, the antibody of the invention competes with an antibody comprising the three CDRs of the heavy chain of mAb15 set forth in SEQ ID NO:13 and the three CDRs of the light chain of mAb15 set forth in SEQ ID NO:14. In some embodiments, the antibody competes with an antibody comprising the three CDRs of the heavy chain of mAb15 set forth in SEQ ID NO:13 and the three CDRs of the light chain of mAb15 set forth in SEQ ID NOs:14 such that less than about any one of 90%, 80%, 70%, 60%, 50%, 40%, 30%, 20%, 10%, or 1% of an antibody comprising the three CDRs of the heavy chain of mAb15 set forth in SEQ ID NO:13 and the three CDRs of the light chain of mAb15 set forth in SEQ ID NO:14 binds TIM-3 in the presence of the antibody of the invention. In some embodiments, the antibody that competes with mAb15 (or an antibody comprising the six CDRs of mAb15) stimulates the secretion of one or more of IL-1β, TNFα, IL-6, GM-CSF or IL-12. In some embodiments, the antibody that competes with mAb15 (or an antibody comprising the six CDRs of mAb15) increases the secretion of one or more of IL-1β, TNFα, IL-6, GM-CSF or IL-12. In some embodiments, the antibody that competes with mAb15 (or an antibody comprising the six CDRs of mAb15) suppresses secretion of one or more of IL-10, CCL2, CCL3, CCL4 or CCL5. In some embodiments, the antibody that competes with mAb15 (or an antibody comprising the six CDRs of mAb15) reduces secretion of one or more of IL-10, CCL2, CCL3, CCL4 or CCL5.

In some embodiments, the invention provides methods of stimulating the secretion of one or more myeloid-associated cytokines in an individual comprising administering to the individual a therapeutically effective amount of antibody that binds TIM-3 comprising the three CDRs of the heavy chain of mAb91 set forth in SEQ ID NO:72 and the three CDRs of the light chain of mAb91 set forth in SEQ ID NO:73. In some embodiments, the antibody comprises three heavy chain CDRs comprising the amino acid sequences SGYYWN (SEQ ID NO:82), YISYDGSNNYNPSLKN (SEQ ID NO:83) and DGPYYYGSSYGYFDV (SEQ ID NO:84) and three light chain CDRs comprising the amino acid sequences RSSKSLLHSNGNTYLY (SEQ ID NO:85), RMSNLAS (SEQ ID NO:86), and MQHLEYPCT (SEQ ID NO:87). In some embodiments, the antibody of the invention competes with an antibody comprising the three CDRs of the heavy chain of mAb91 set forth in SEQ ID NO:72 and the three CDRs of the light chain of mAb91 set forth in SEQ ID NO:73. In some embodiments, the antibody competes with an antibody comprising the three CDRs of the heavy chain of mAb91 set forth in SEQ ID NO:72 and the three CDRs of the light chain of mAb91 set forth in SEQ ID NOs:73 such that less than about any one of 90%, 80%, 70%, 60%, 50%, 40%, 30%, 20%, 10%, or 1% of an antibody comprising the three CDRs of the heavy chain of mAb91 set forth in SEQ ID NO:72 and the three CDRs of the light chain of mAb91 set forth in SEQ ID NO:73 binds TIM-3 in the presence of the antibody of the invention. In some embodiments, the antibody that competes with mAb91 (or an antibody comprising the six CDRs of mAb91) stimulates the secretion of one or more of IL-1β, TNFα, IL-6, GM-CSF or IL-12. In some embodiments, the antibody that competes with mAb91 (or an antibody comprising the six CDRs of mAb91) increases the secretion of one or more of IL-1β, TNFα, IL-6, GM-CSF or IL-12. In some embodiments, the antibody that competes with mAb91 (or an antibody comprising the six CDRs of mAb91) suppresses secretion of one or more of IL-10, CCL2, CCL3, CCL4 or CCL5. In some embodiments, the antibody that competes with mAb91 (or an antibody comprising the six CDRs of mAb91) reduces secretion of one or more of IL-10, CCL2, CCL3, CCL4 or CCL5.

In some embodiments, binding of an anti-TIM-3 antibody disclosed herein to TIM-3 leads to the preferential activation of macrophages and/or the preferential secretion of pro-inflammatory myeloid-associated cytokines. In some embodiments, an M1 macrophage is a macrophage that expresses at least one, at least two, at least three, at least four, at least five, at least six, or seven proteins selected from CD86, CD80, MHCII^{HIGH}, IL-1R, TLR2, TLR4, and iNOS on its surface. In some embodiments, an M1 macrophage is a macrophage that expresses iNOS on its surface. In some embodiments, an M1 macrophage is a macrophage that secretes at least one, at least two, at least three, at least four, or five cytokines selected from TNF-α, IL-1β, IL-6, IL-12, and IL-23. In some embodiments, an M1 macrophage is a macrophage that secretes at least one, at least two, or three cytokines selected from TNF-α, IL-1, and IL-23. In some embodiments, an M1 macrophage is a macrophage that secretes at least one, at least two, at least three, at least four, at least five, at least six, at least seven, or eight chemokines selected from CCL10, CCL11, CCL5, CCL8 CCL9, CCL2, CCL3, and CCL4. In some embodiments, a method comprises increasing at least one, at least two, at least three, at least four, or at least five markers associated with M1 macrophages. In some embodiments, the markers associated with M1 macrophages are selected from CD86, CD80, MHCII^{HIGH}, IL-1R, TLR2, TLR4, iNOS, TNF-α, IL-1β, IL-6, IL-12, IL-2, CCL10, CCL11, CCL5, CCL8 CCL9, CCL2, CCL3, and CCL4. In some embodiments, the markers are selected from iNOS, TNF-α, IL-1, and IL-23.

In some embodiments, an M2 macrophage is a macrophage that expresses at least one, at least two, at least three, at least four, or five proteins selected from CD163, MHCII^{LOW}, CD206, IL-4R, and IL-1RII on its surface. In some embodiments, an M2 macrophage is a macrophage that expresses at least one or both proteins selected from CD206 and IL-4R on its surface. In some embodiments, an M2 macrophage is a macrophage that secretes TGF-β and/or IL-10. In some embodiments, an M2 macrophage is a macrophage that secretes TGF-β and IL-10. In some embodiments, an M2 macrophage is a macrophage that secretes at least one, at least two, at least three, at least four, at least five, or six chemokines selected from CCL17, CCL22, CCL24, CCL1, CXCL10, and CXCL16. In some embodiments, a method comprises reducing at least one, at least two, at least three, at least four, or at least five markers associated with M2 macrophages. In some embodiments, the markers associated with M2 macrophages are selected from CD163, MHCII^{LOW}, CD206, IL-4R, IL-1RII, TGF-β, IL-10, CCL17, CCL22, CCL24, CCL1, CXCL10, and CXCL16. In some embodiments, the markers are selected from CD206, IL-4R, TGF-β, and IL-10.

Antibodies and compositions comprising antibodies are provided for use in methods of treatment for humans or animals. Methods of treating disease comprising administering antibodies that inhibit the interaction of TIM-3 and LILRB2 are also provided. Nonlimiting exemplary diseases that can be treated with antibodies that inhibit the interaction of TIM-3 and LILRB2 include, but are not limited to, various forms of cancer.

The antibodies that inhibit the interaction of TIM-3 and LILRB2 can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of an antibody is administered to a subject one or more times. In some embodiments, an effective dose of an antibody is administered to the subject once a month, more than once a month, such as, for example, every two months or every three months. In some embodiments, an effective dose of an antibody is administered less than once a month, such as, for example, every two weeks or every week. An effective dose of an antibody is administered to the subject at least once. In some embodiments, the effective dose of an antibody may be administered multiple times, including for periods of at least a month, at least six months, or at least a year.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated. In general, antibodies that inhibit the interaction of TIM-3 and LILRB2 may be administered in an amount in the range of about 0.05 mg/kg body weight to about 100 mg/kg body weight per dose. In some embodiments, antibodies that inhibit the interaction of TIM-3 and LILRB2 may be administered in an amount in the range of about 10 μg/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 0.05 mg/kg body weight to about 20 mg/kg body weight per dose. In some embodiments, antibodies may be administered in an amount in the range of about 0.05 mg/kg body weight to about 10 mg/kg body weight per dose. In some embodiments, antibodies may be administered in an amount in the range of about 5 mg/kg body weight or lower, for example less than 4, less than 3, less than 2, or less than 1 mg/kg of the antibody.

In some embodiments, pharmaceutical compositions are administered in an amount effective for treatment of cancer and/or encouraging T-cell proliferation. The therapeutically effective amount is typically dependent on the weight of the subject being treated, his or her physical or health condition, the extensiveness of the condition to be treated, or the age of the subject being treated. In general, antibodies that inhibit the interaction of TIM-3 and LILRB2 may be administered in an amount in the range of about 10 μg/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 0.5 mg/kg body weight to about 5 mg/kg body weight per dose.

Below is an outline of further embodiments and particulars for performing the above noted methods, as well as further methods. The placement of the embodiments below is to clarify that it is contemplated that any of the embodiments provided herein can be combined with any of the other aspects listed herein.

In some embodiments, the antibody that inhibits the interaction of TIM-3 and LILRB2 is given concurrently with a second therapeutic agent, for example, a PD-1 therapy. Examples of PD-1 therapy include Nivolumab ((Bristol-Myers Squibb, OPDIVO®, BMS-936558, MDX-1106, ONO-4538); Pidilizumab (CureTech, CT-011), Lambrolizumab/pembrolizumab (Merck, KEYTRUDA®, MK-3475); durvalumab (Medimmune/AstraZeneca, MEDI-4736); RG7446/MPDL3280A (Genentech/Roche); MSB-0010718C (Merck Serono); AMP-224 (Amplimmune); BMS-936559; AMP-514 (Amplimmune; MDX-1105 (Merck); TSR-042 (Tesaro/AnaptysBio, ANB-011); STI-A1010 (Sorrento Therapeutics); STI-A1110 (Sorrento Therapeutics); and other antibodies that are directed against programmed death-1 (PD-1) or programmed death ligand 1 (PD-L1).

In some embodiments, the therapeutic treatment involving the use of an antibody that inhibits the interaction of TIM-3 and LILRB2 is achieved by T-cell modulation. In some embodiments, increasing T-cell proliferation inhibits growth of the cancer. In some embodiments, inhibition of growth of the cancer is further enhanced by antibody-dependent cell-mediated cytotoxicity (ADCC). In some embodiments, inhibition of growth of the cancer does not occur by ADCC. In some embodiments, inhibition of growth of the cancer does not occur by ADC (antibody-drug conjugate). In some embodiments, inhibition of growth of the cancer occurs by allowing the host's immune system to properly act on the cancer. In some embodiments, T-cell proliferation is a result of T-cell activation. In some embodiments, the use of an antibody that inhibits the interaction of TIM-3 and LILRB2 based therapy in one of the methods provided herein restores the subject's endogenous immune response to the cancer. In some embodiments, the subject's endogenous immune response is sufficient to slow the progression of or remove the cancer. In some embodiments, any of the methods provided herein can further comprise assaying an amount of TIM-3 present in a cancer in the subject. In some embodiments, the subject can be identified as one that has previously received no significant improvement from a PD-1 therapy. In some embodiments, the subject is one that received a detectable level of improvement from the PD-1 therapy, but an additional amount of improvement is beneficial or desired for the subject. In some embodiments, tumors of the patient express low levels of PD-L1. In some embodiments, tumors of the patient express high levels of PD-L1. In some embodiments, tumors of the patient express low levels of PD-L1 and high levels of TIM-3. In some embodiments, tumors of the patient express high levels of PD-L1 and high levels of TIM-3. Any method of detecting the level of a protein in a sample is contemplated. One skilled in the art can select a suitable method depending on the type of sample being analyzed and the identity and number of proteins being detected. Nonlimiting exemplary such methods include immunohistochemistry, ELISA, Western blotting, multiplex analyte detection (using, for example, Luminex technology), mass spectrometry, etc. Similarly, any method of detecting the level of an mRNA in a sample is contemplated. One skilled in the art can select a suitable method depending on the type of sample being analyzed and the identity and number of mRNAs being detected. Nonlimiting exemplary such methods include RT-PCR, quantitative RT-PCR and microarray-based methods, etc. In some embodiment, PD-L1 level can measured using PD-L1 IHC assay with PD-L1 IHC 22C3 pharmDx test (Dako Inc., Carpinteria, Calif.).

In some embodiments, the method of treatment or inducing T-cell proliferation described herein can further include administering: radiation therapy, chemotherapy, vaccination, targeted tumor therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent.

As will be appreciated by one of skill in the art, in some embodiments, any of the herein disclosed methods can be used separately or in combination for one or more of: treatment of cancer, increasing production of cytokines and/or increasing cytokine secretion, and/or increasing T-cell proliferation. Thus, any of the methods directed to any of these three areas (treatment of cancer, increasing production of cytokines and/or increasing cytokine secretion, and/or increasing T-cell proliferation) is contemplated as being alternatives methods for the other two areas (treatment of cancer, increasing production of cytokines and/or increasing cytokine secretion, and/or increasing T-cell proliferation).

In some embodiments, the methods provided herein allow for one to increase production of cytokines and/or increase cytokine secretion. In some embodiments, any cytokine level can be increased. In some embodiments, the cytokine that has its level increased is at least one of IL-1β, TNFα and/or IL-12.

In some embodiments, any of the methods provided herein can be performed by an antagonist TIM-3 antibody.

Antibodies that Modulate the Interaction of TIM-3 and LILRB2

The invention provides antibodies than modulate the interaction of TIM-3 and LILRB2 expressed on immune cells such that cells of myeloid lineage, particularly macrophages, are stimulated to secrete pro-inflammatory cytokines. In some embodiments the antibody inhibits the interaction of TIM-3 and LILRB2; for example, by inhibiting the binding of TIM-3 to LILRB2.

In some embodiments, the antibody specifically binds TIM-3 such that binding of TIM-3 to LILRB2 is inhibited. In some embodiments, the binding of TIM-3 to LILRB2 is inhibited by at least about any one of 1%, 5%, 10%, 20%, 30%, 40%, 50%, 60%, 70%, 80%, 90% or 100%. In some embodiments, the binding of TIM-3 to LILRB2 is inhibited by any one of about 1% to about 10%, about 10% to about 25%, 10% to about 50%, 10% to about 75%, about 10% to about 100%, about 25% to about 50%, about 25% to about 75%, about 25% to about 100%, about 50% to about 75%, about 50% to about 100%, or about 75% to about 100%.

In some embodiments, the antibody specifically competes with LILRB2 for binding to TIM-3. Methods to determine competition for binding are known in the art; for example, by using the OctetRED 96 system as demonstrated in Example 7 below. Other examples include but are not limited to competitive binding in a flow-cytometric assay to a molecule displayed on the surface of a cell or bead or by ELISA where the molecule is bound to a plate and competition is demonstrated by competitive binding. In some embodiments, the antibody competes with LILRB2 for binding to TIM-3 such that binding of LILRB2 to TIM-3 is inhibited by at least about any one of 1%, 5%, 10%, 20%, 30%, 40%, 50%, 60%, 70%, 80%, 90% or 100%. In some embodiments, the binding of TIM-3 to LILRB2 is inhibited by any one of about 1% to about 10%, about 10% to about 25%, 10% to about 50%, 10% to about 75%, about 10% to about 100%, about 25% to about 50%, about 25% to about 75%, about 25% to about 100%, about 50% to about 75%, about 50% to about 100%, or about 75% to about 100%.

In some embodiments, the antibody specifically binds LILRB2 such that binding of LILRB2 to TIM-3 is inhibited. In some embodiments, the binding of LILRB2 to TIM-3 is inhibited by at least about any one of 1%, 5%, 10%, 20%, 30%, 40%, 50%, 60%, 70%, 80%, 90% or 100%. In some embodiments, the binding of LILRB2 to TIM-3 is inhibited by any one of about 1% to about 10%, about 10% to about 25%, 10% to about 50%, 10% to about 75%, about 10% to about 100%, about 25% to about 50%, about 25% to about 75%, about 25% to about 100%, about 50% to about 75%, about 50% to about 100%, or about 75% to about 100%.

In some embodiments, the antibody specifically competes with TIM-3 for binding to LILRB2. Methods to determine competition for binding are known in the art; for example, by using the OctetRED 96 system as demonstrated in Example 7 below. In some embodiments, the antibody competes with TIM-3 for binding to LILRB2 such that binding of TIM-3 to LILRB2 is inhibited by at least about any one of 1%, 5%, 10%, 20%, 30%, 40%, 50%, 60%, 70%, 80%, 90% or 100%. In some embodiments, the binding of LILRB2 to TIM-3 is inhibited by any one of about 1% to about 10%, about 10% to about 25%, 10% to about 50%, 10% to about 75%, about 10% to about 100%, about 25% to about 50%, about 25% to about 75%, about 25% to about 100%, about 50% to about 75%, about 50% to about 100%, or about 75% to about 100%.

In some embodiments, the antibody that inhibits the interaction of TIM-3 and LILRB2 wherein the TIM-3 is from a human, a mouse or a rat. In some embodiments, the TIM-3 is an isoform 1 TIM-3. In other embodiments, the TIM-3 is an isoform 2 TIM-3. In some embodiments, the TIM-3 comprises the amino acid sequence set forth in SEQ ID NO:1, SEQ ID NO:3 or SEQ ID NO:9. In some embodiments, the TIM-3 is a variant of TIM-3 isoform 1 or TIM-3 isoform 2. In some embodiments, the TIM-3 comprises about 1, 2, 3, 4, 5, 6, 7, 8, 9, 10, 10-25 or 25-50 amino acid substitutions of the amino acid sequence set forth in SEQ ID NO:1, SEQ ID NO:3 or SEQ ID NO:9, while maintaining TIM-3 activity. In some embodiments, the TIM-3 comprises an amino acid sequence that is at least about any of 60%, 70%, 80%, 85%, 90%, 95%, or 99% identical to the amino acid sequence set forth in SEQ ID NO:1, SEQ ID NO:3 or SEQ ID NO:9.

In some embodiments, the antibody that inhibits the interaction of TIM-3 and LILRB2 wherein the LILRB2 is from a human. In some embodiments, the LILRB2 is a variant 1 LILRB2. In other embodiments, the LILRB2 is a variant 2 LILRB2. In some embodiments, the LILRB2 comprises the amino acid sequence set forth in SEQ ID NO:5 or SEQ ID NO:7. In some embodiments, the LILRB2 is a variant of LILRB2. In some embodiments, the LILRB2 comprises about 1, 2, 3, 4, 5, 6, 7, 8, 9, 10, 10-25 or 25-50 amino acid substitutions of the amino acid sequence set forth in SEQ ID NO:5 or SEQ ID NO:7, while maintaining LILRB2 activity. In some embodiments, the LILRB2 comprises an amino acid sequence that is at least about any of 60%, 70%, 80%, 85%, 90%, 95%, or 99% identical to the amino acid sequence set forth in SEQ ID NO:5 or SEQ ID NO:7.

In some aspects, the invention provides an antibody that modulates the interaction of TIM-3 and LILRB2. In some embodiments, the modulation of the interaction of TIM-3 and LILRB2 is an inhibition of the interaction of TIM-3 and LILRB2; for example, by inhibiting the binding of TIM-3 and LILRB2. The inhibition of the interaction of TIM-3 and LILRB2 may lead to the activation of cells of monocyte/macrophage lineages; e.g., macrophages, which leads to the secretion of pro-inflammatory cytokines. In some embodiments, the antibody binds TIM-3. In other embodiments, the antibody binds LILRB2. In some embodiments, binding of the antibody to TIM-3 or LILRB2 leads to the preferential activation of macrophages. In some embodiments, binding of the antibody to TIM-3 or LILRB2 leads to the preferential activation of dendritic cells. In some embodiments, binding of the antibody to TIM-3 or LILRB2 leads to the preferential activation of macrophages and dendritic cells. In some embodiments, binding of the antibody to TIM-3 or LILRB2 leads to the preferential secretion of pro-inflammatory cytokines by macrophages. In some embodiments, binding of the antibody to TIM-3 or LILRB2 leads to the preferential secretion of pro-inflammatory cytokines by dendritic cells. In some embodiments, binding of the antibody to TIM-3 or LILRB2 leads to the preferential secretion of pro-inflammatory cytokines by macrophages and dendritic cells. In some embodiments, binding of the antibody to TIM-3 or LILRB2 leads to the preferential secretion of pro-inflammatory cytokines that interact with macrophages. In some embodiments, binding of the antibody to TIM-3 or LILRB2 leads to the preferential secretion of pro-inflammatory cytokines that interact with dendritic cells. In some embodiments, binding of the antibody to TIM-3 or LILRB2 leads to the preferential secretion of pro-inflammatory cytokines that interact with macrophages and dendritic cells. In some embodiments, the individual is human.

In some embodiments, the pro-inflammatory cytokine is IL-12, TNFα, IL-1β, GM-CSF, or IL-6. In some embodiments, any one, any two, any three, any four or all five cytokines are secreted by monocytes or macrophages following administration of an antibody that inhibits the interaction of TIM-3 and LILRB2. In some embodiments, one or more of pro-inflammatory cytokines IL-12, TNFα, IL-1β, GM-CSF or IL-6 is secreted following administration of an antibody that inhibits the interaction of TIM-3 and LILRB2. In some embodiments, secretion of pro-inflammatory cytokines following administration of an antibody of the invention is increased compared to secretion of pro-inflammatory cytokines following administration of antibody F38-2E2. In some embodiments, the secretion of pro-inflammatory cytokines (e.g., IL-12, TNFα, IL-1β, GM-CSF or IL-6) is at least about any of 2-fold, 3-fold, 4-fold, 5-fold, 6-fold, 7-fold, 8-fold, 9-fold, or 10-fold following administration of an antibody of the invention compared to secretion of pro-inflammatory cytokines following administration of antibody F38-2E2.

In some embodiments, the antibody suppresses secretion of a cytokine (e.g., reduces secretion of a cytokine). In some embodiments, the cytokine is IL-10, CCL2, CCL3, CCL4 or CCL5. In some embodiments, secretion of any one, any two, any three, any four or all five cytokines are inhibited following administration of an antibody that inhibits the interaction of TIM-3 and LILRB2. In some embodiments, secretion of cytokines following administration of an antibody of the invention is suppressed compared to secretion of cytokines following administration of antibody F38-2E2. In some embodiments, secretion of cytokines following administration of an antibody of the invention is reduced compared to secretion of cytokines following administration of antibody F38-2E2. In some embodiments, the secretion of cytokines (e.g., IL-10, CCL2, CCL3, CCL4 or CCL5) is at least about any of 2-fold, 3-fold, 4-fold, 5-fold, 6-fold, 7-fold, 8-fold, 9-fold, or 10-fold less following administration of an antibody of the invention compared to secretion of pro-inflammatory cytokines following administration of antibody F38-2E2.

In some embodiments, the invention provides antibodies that stimulate the secretion of a myeloid-associated cytokine in an individual with cancer; for example, increases the secretion of a myeloid-associated cytokine in an individual with cancer. In some embodiments, the cytokines are secreted in a tumor; for example, pro-inflammatory cytokines are secreted by a cell located in or near a tumor. In some embodiments, the individual is human.

In some embodiments, the invention provides antibody mAb15. In some embodiments, the antibody is a humanized mAb15. In some embodiments, the antibody binds the same epitope as antibody mAb15. In some embodiments. In some embodiments, the invention provides antibodies that compete with antibody mAb15. In some embodiments, the antibody competes with mAb15 such that less than about any one of 90%, 80%, 70%, 60%, 50%, 40%, 30%, 20%, 10%, or 1% of mAb15 binds TIM-3 in the presence of the antibody of the invention.

In some embodiments, the invention provides an antibody that binds TIM-3 comprising the three CDRs of the heavy chain of mAb15 set forth in SEQ ID NO:12 and the three CDRs of the light chain of mAb15 set forth in SEQ ID NO:14. In some embodiments, the antibody comprises three heavy chain CDRs comprising the amino acid sequences GYGVT (SEQ ID NO:59), MIWGDGNTDYNSGLKS (SEQ ID NO:80) and SYYYGPPDY (SEQ ID NO:81). In some embodiments, the antibody comprises three light chain CDRs comprising the amino acid sequences KSSQSLLNSRSQKNYLA (SEQ ID NO:88), FASTRES (SEQ ID NO:89) and HQHYNTPYT (SEQ ID NO:20). In some embodiments, the antibody comprises three heavy chain CDRs comprising the amino acid sequences GFSLTGYG (SEQ ID NO:15), IWGDGNT (SEQ ID NO:16) and ARSYYYGPPDY (SEQ ID NO:17). In some embodiments, the antibody comprises three light chain CDRs comprising the amino acid sequences QSLLNSRSQKNY (SEQ ID NO:18), FAS (SEQ ID NO:19) and HQHYNTPYT (SEQ ID NO:20). In some embodiments, the antibody of the invention competes with an antibody comprising the three CDRs of the heavy chain of mAb15 set forth in SEQ ID NO:12 and the three CDRs of the light chain of mAb15 set forth in SEQ ID NO:14. In some embodiments, the antibody competes with an antibody comprising the three CDRs of the heavy chain of mAb15 set forth in SEQ ID NO:12 and the three CDRs of the light chain of mAb15 set forth in SEQ ID NO:14 such that less than about any one of 90%, 80%, 70%, 60%, 50%, 40%, 30%, 20%, 10%, or 1% of an antibody comprising the three CDRs of the heavy chain of mAb15 set forth in SEQ ID NO:12 and the three CDRs of the light chain of mAb15 set forth in SEQ ID NO:1 binds TIM-3 in the presence of the antibody of the invention.

In some embodiments, the invention provides an antibody that binds TIM-3 comprising the three CDRs of the heavy chain of mAb13 set forth in SEQ ID NO:21 and the three CDRs of the light chain of mAb13 set forth in SEQ ID NO:22. In some embodiments, the antibody comprises three heavy chain CDRs comprising the amino acid sequences GYTFTDYYIN (SEQ ID NO:27), WIYPGSGNTKYNEKFKG (SEQ ID NO:28) and GGKYYAMDY (SEQ ID NO:29) and three light chain CDRs comprising the amino acid sequences KASQSVGNNVA (SEQ ID NO:30), YASNRYT (SEQ ID NO:31), and QQDYSSPYT (SEQ ID NO:32). In some embodiments, the antibody of the invention competes with an antibody comprising the three CDRs of the heavy chain of mAb13 set forth in SEQ ID NO:21 and the three CDRs of the light chain of mAb13 set forth in SEQ ID NO:22. In some embodiments, the antibody competes with an antibody comprising the three CDRs of the heavy chain of mAb13 set forth in SEQ ID NO:21 and the three CDRs of the light chain of mAb13 set forth in SEQ ID NO:22 such that less than about any one of 90%, 80%, 70%, 60%, 50%, 40%, 30%, 20%, 10%, or 1% of an antibody comprising the three CDRs of the heavy chain of mAb13 set forth in SEQ ID NO:21 and the three CDRs of the light chain of mAb13 set forth in SEQ ID NO:22 binds TIM-3 in the presence of the antibody of the invention. In some embodiments, the antibody that competes with mAb13 (or an antibody comprising the six CDRs of mAb13) stimulates the secretion of one or more of IL-1β, TNFα, IL-6, GM-CSF or IL-12. In some embodiments, the antibody that competes with mAb13 (or an antibody comprising the six CDRs of mAb13) increases the secretion of one or more of IL-1β, TNFα, IL-6, GM-CSF or IL-12. In some embodiments, the antibody that competes with mAb13 (or an antibody comprising the six CDRs of mAb13) suppresses secretion of one or more of IL-10, CCL2, CCL3, CCL4 or CCL5. In some embodiments, the antibody that competes with mAb13 (or an antibody comprising the six CDRs of mAb13) reduces secretion of one or more of IL-10, CCL2, CCL3, CCL4 or CCL5.

In some embodiments, the invention provides an antibody that binds TIM-3 comprising the three CDRs of the heavy chain of mAb17 set forth in SEQ ID NO:23 and the three CDRs of the light chain of mAb17 set forth in SEQ ID NO:24. In some embodiments, the antibody comprises three heavy chain CDRs comprising the amino acid sequences NYGMS (SEQ ID NO:91), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QNSHSFPPT (SEQ ID NO:38). In some embodiments, the antibody comprises three heavy chain CDRs comprising the amino acid sequences GFTFSNYGMS (SEQ ID NO:33), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QNSHSFPPT (SEQ ID NO:38). In some embodiments, the antibody of the invention competes with an antibody comprising the three CDRs of the heavy chain of mAb17 set forth in SEQ ID NO:23 and the three CDRs of the light chain of mAb17 set forth in SEQ ID NO:24. In some embodiments, the antibody competes with an antibody comprising the three CDRs of the heavy chain of mAb17 set forth in SEQ ID NO:23 and the three CDRs of the light chain of mAb17 set forth in SEQ ID NO:24 such that less than about any one of 90%, 80%, 70%, 60%, 50%, 40%, 30%, 20%, 10%, or 1% of an antibody comprising the three CDRs of the heavy chain of mAb17 set forth in SEQ ID NO:23 and the three CDRs of the light chain of mAb17 set forth in SEQ ID NO:24 binds TIM-3 in the presence of the antibody of the invention. In some embodiments, the antibody that competes with mAb17 (or an antibody comprising the six CDRs of mAb17) stimulates the secretion of one or more of IL-1β, TNFα, IL-6, GM-CSF or IL-12. In some embodiments, the antibody that competes with mAb17 (or an antibody comprising the six CDRs of mAb17) increases the secretion of one or more of IL-1β, TNFα, IL-6, GM-CSF or IL-12. In some embodiments, the antibody that competes with mAb17 (or an antibody comprising the six CDRs of mAb17) suppresses secretion of one or more of IL-10, CCL2, CCL3, CCL4 or CCL5. In some embodiments, the antibody that competes with mAb17 (or an antibody comprising the six CDRs of mAb17) reduces secretion of one or more of IL-10, CCL2, CCL3, CCL4 or CCL5.

In some embodiments, the invention provides an antibody that binds TIM-3 comprising the three CDRs of the heavy chain of mAb22 set forth in SEQ ID NO:25 and the three CDRs of the light chain of mAb22 set forth in SEQ ID NO:26. In some embodiments, the antibody comprises three heavy chain CDRs comprising the amino acid sequences NHGMS (SEQ ID NO:97), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QHSHSFPPT (SEQ ID NO:40). In some embodiments, the antibody comprises three heavy chain CDRs comprising the amino acid sequences GFTFSNHGMS (SEQ ID NO:39), TISSGGSNTYFPDSVKG (SEQ ID NO:34), and HGTSMIKEWFAY (SEQ ID NO:35) and three light chain CDRs comprising the amino acid sequences RASQSIGDYLH (SEQ ID NO:36), YASQSIS (SEQ ID NO:37), and QHSHSFPPT (SEQ ID NO:40). In some embodiments, the antibody of the invention competes with an antibody comprising the three CDRs of the heavy chain of mAb22 set forth in SEQ ID NO:25 and the three CDRs of the light chain of mAb22 set forth in SEQ ID NO:26. In some embodiments, the antibody competes with an antibody comprising the three CDRs of the heavy chain of mAb22 set forth in SEQ ID NO:25 and the three CDRs of the light chain of mAb22 set forth in SEQ ID NO:26 such that less than about any one of 90%, 80%, 70%, 60%, 50%, 40%, 30%, 20%, 10%, or 1% of an antibody comprising the three CDRs of the heavy chain of mAb22 set forth in SEQ ID NO:25 and the three CDRs of the light chain of mAb22 set forth in SEQ ID NO:26 binds TIM-3 in the presence of the antibody of the invention. In some embodiments, the antibody that competes with mAb22 (or an antibody comprising the six CDRs of mAb22) stimulates the secretion of one or more of IL-1β, TNFα, IL-6, GM-CSF or IL-12. In some embodiments, the antibody that competes with mAb22 (or an antibody comprising the six CDRs of mAb22) increases the secretion of one or more of IL-1β, TNFα, IL-6, GM-CSF or IL-12. In some embodiments, the antibody that competes with mAb22 (or an antibody comprising the six CDRs of mAb22) suppresses secretion of one or more of IL-10, CCL2, CCL3, CCL4 or CCL5. In some embodiments, the antibody that competes with mAb22 (or an antibody comprising the six CDRs of mAb22) reduces secretion of one or more of IL-10, CCL2, CCL3, CCL4 or CCL5.

In some embodiments, the invention provides an antibody that binds TIM-3 comprising the three CDRs of the heavy chain of mAb58 set forth in SEQ ID NO:53 and the three CDRs of the light chain of mAb58 set forth in SEQ ID NO:54. In some embodiments, the antibody comprises three heavy chain CDRs comprising the amino acid sequences TYGMS (SEQ ID NO:55), WINTYSGAPTYADDFKG (SEQ ID NO:56) and KPPHYYVNSFDY (SEQ ID NO:57) and three light chain CDRs comprising the amino acid sequences RASQSISDYLH (SEQ ID NO:58), YASQSIS (SEQ ID NO:37), and QNGHSFPYT (SEQ ID NO:60). In some embodiments, the antibody of the invention competes with an antibody comprising the three CDRs of the heavy chain of mAb58 set forth in SEQ ID NO:53 and the three CDRs of the light chain of mAb58 set forth in SEQ ID NO:54. In some embodiments, the antibody competes with an antibody comprising the three CDRs of the heavy chain of mAb58 set forth in SEQ ID NO:53 and the three CDRs of the light chain of mAb58 set forth in SEQ ID NOs:54 such that less than about any one of 90%, 80%, 70%, 60%, 50%, 40%, 30%, 20%, 10%, or 1% of an antibody comprising the three CDRs of the heavy chain of mAb58 set forth in SEQ ID NO:53 and the three CDRs of the light chain of mAb58 set forth in SEQ ID NO:54 binds TIM-3 in the presence of the antibody of the invention. In some embodiments, the antibody that competes with mAb58 (or an antibody comprising the six CDRs of mAb58) stimulates the secretion of one or more of IL-1β, TNFα, IL-6, GM-CSF or IL-12. In some embodiments, the antibody that competes with mAb58 (or an antibody comprising the six CDRs of mAb58) increases the secretion of one or more of IL-1β, TNFα, IL-6, GM-CSF or IL-12. In some embodiments, the antibody that competes with mAb58 (or an antibody comprising the six CDRs of mAb58) suppresses secretion of one or more of IL-10, CCL2, CCL3, CCL4 or CCL5. In some embodiments, the antibody that competes with mAb58 (or an antibody comprising the six CDRs of mAb58) reduces secretion of one or more of IL-10, CCL2, CCL3, CCL4 or CCL5.

In some embodiments, the invention provides an antibody that binds TIM-3 comprising the three CDRs of the heavy chain of mAb48 set forth in SEQ ID NO:70 and the three CDRs of the light chain of mAb48 set forth in SEQ ID NO:71. In some embodiments, the antibody comprises three heavy chain CDRs comprising the amino acid sequences DYYIN (SEQ ID NO:90), WIYPGSGNTKYNEKFKG (SEQ ID NO:28) and DRFDY (SEQ ID NO:92) and three light chain CDRs comprising the amino acid sequences SASSGVSSSYLY (SEQ ID NO:93), STSNLAS (SEQ ID NO:94), and HQWSNSPYT (SEQ ID NO:95). In some embodiments, the antibody of the invention competes with an antibody comprising the three CDRs of the heavy chain of mAb48 set forth in SEQ ID NO:70 and the three CDRs of the light chain of mAb48 set forth in SEQ ID NO:71. In some embodiments, the antibody competes with an antibody comprising the three CDRs of the heavy chain of mAb48 set forth in SEQ ID NO:70 and the three CDRs of the light chain of mAb48 set forth in SEQ ID NOs:71 such that less than about any one of 90%, 80%, 70%, 60%, 50%, 40%, 30%, 20%, 10%, or 1% of an antibody comprising the three CDRs of the heavy chain of mAb48 set forth in SEQ ID NO:70 and the three CDRs of the light chain of mAb48 set forth in SEQ ID NO:71 binds TIM-3 in the presence of the antibody of the invention. In some embodiments, the antibody that competes with mAb48 (or an antibody comprising the six CDRs of mAb48) stimulates the secretion of one or more of IL-1β, TNFα, IL-6, GM-CSF or IL-12. In some embodiments, the antibody that competes with mAb48 (or an antibody comprising the six CDRs of mAb48) increases the secretion of one or more of IL-1β, TNFα, IL-6, GM-CSF or IL-12. In some embodiments, the antibody that competes with mAb48 (or an antibody comprising the six CDRs of mAb48) suppresses secretion of one or more of IL-10, CCL2, CCL3, CCL4 or CCL5. In some embodiments, the antibody that competes with mAb48 (or an antibody comprising the six CDRs of mAb48) reduces secretion of one or more of IL-10, CCL2, CCL3, CCL4 or CCL5.

In some embodiments, the invention provides an antibody that binds TIM-3 comprising the three CDRs of the heavy chain of mAb91 set forth in SEQ ID NO:72 and the three CDRs of the light chain of mAb91 set forth in SEQ ID NO:73. In some embodiments, the antibody comprises three heavy chain CDRs comprising the amino acid sequences SGYYWN (SEQ ID NO:82), YISYDGSNNYNPSLKN (SEQ ID NO:83) and DGPYYYGSSYGYFDV (SEQ ID NO:84) and three light chain CDRs comprising the amino acid sequences RSSKSLLHSNGNTYLY (SEQ ID NO:85), RMSNLAS (SEQ ID NO:86), and MQHLEYPCT (SEQ ID NO:87). In some embodiments, the antibody of the invention competes with an antibody comprising the three CDRs of the heavy chain of mAb91 set forth in SEQ ID NO:72 and the three CDRs of the light chain of mAb91 set forth in SEQ ID NO:73. In some embodiments, the antibody competes with an antibody comprising the three CDRs of the heavy chain of mAb91 set forth in SEQ ID NO:72 and the three CDRs of the light chain of mAb91 set forth in SEQ ID NOs:73 such that less than about any one of 90%, 80%, 70%, 60%, 50%, 40%, 30%, 20%, 10%, or 1% of an antibody comprising the three CDRs of the heavy chain of mAb91 set forth in SEQ ID NO:72 and the three CDRs of the light chain of mAb91 set forth in SEQ ID NO:73 binds TIM-3 in the presence of the antibody of the invention. In some embodiments, the antibody that competes with mAb91 (or an antibody comprising the six CDRs of mAb91) stimulates the secretion of one or more of IL-1β, TNFα, IL-6, GM-CSF or IL-12. In some embodiments, the antibody that competes with mAb91 (or an antibody comprising the six CDRs of mAb91) increases the secretion of one or more of IL-1β, TNFα, IL-6, GM-CSF or IL-12. In some embodiments, the antibody that competes with mAb91 (or an antibody comprising the six CDRs of mAb91) suppresses secretion of one or more of IL-10, CCL2, CCL3, CCL4 or CCL5. In some embodiments, the antibody that competes with mAb91 (or an antibody comprising the six CDRs of mAb91) reduces secretion of one or more of IL-10, CCL2, CCL3, CCL4 or CCL5.

In some embodiments, the antibody that inhibits the interaction of TIM-3 and LILRB2 is a monoclonal antibody; for example, a monoclonal antibody that binds TIM-3 or a monoclonal antibody that binds LILRB2. In some embodiments, the monoclonal antibody is chimeric antibody, a humanized antibody or a human antibody. In some embodiments, the monoclonal antibody is an antigen binding fragment; for example, a Fab, a Fab′, an Fv, an scFv, or a (Fab′)2 fragment.

In some embodiments, the antibody that inhibits the interaction of TIM-3 and LILRB2 comprises a heavy chain variable region and a light chain variable region. In some embodiments, the antibody comprises at least one heavy chain comprising a heavy chain variable region and at least a portion of a heavy chain constant region, and at least one light chain comprising a light chain variable region and at least a portion of a light chain constant region. In some embodiments, the antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the antibody that comprises the three heavy chain CDRs. In some embodiments, the light chain is the region of the antibody that comprises the three light chain CDRs.

In some embodiments, an antibody is a chimeric antibody. Certain chimeric antibodies are described, for example, in U.S. Pat. No. 4,816,567; and Morrison et al., (1984) Proc. Natl. Acad. Sci. USA, 81 :6851-6855 (1984)). In one example, a chimeric antibody comprises a non-human variable region (for example, a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, a chimeric antibody described herein comprises a human IgG constant region. In some embodiments, a chimeric antibody described herein comprises a human IgG4 heavy chain constant region. In some embodiments, a chimeric antibody described herein comprises a human IgG4 constant region and a human κ light chain.

As noted above, whether or not effector function is desirable may depend on the particular method of treatment intended for an antibody. Thus, in some embodiments, when effector function is desirable, a chimeric antibody comprising a human IgG1 heavy chain constant region or a human IgG3 heavy chain constant region is selected. In some embodiments, when effector function is not desirable, a chimeric antibody comprising a human IgG4 or IgG2 heavy chain constant region is selected.

In some embodiments, humanized antibodies that inhibit the interaction of TIM-3 and LILRB2 are provided. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151 :2296); framework regions derived from the consensus sequence of human antibodies of a particular subgroup of light or heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271 :22611-22618).

In some embodiments, the antibody that inhibits the interaction of TIM-3 and LILRB2 is a human antibody. Human antibodies can be produced using various techniques known in the art. Human antibodies are described generally in van Dijk and van de Winkel, (2001) Curr. Opin. Pharmacol. 5:368-374 and Lonberg, (2008) Curr. Opin. Immunol. 20:450-459. In some embodiments, the human antibody is not a naturally occurring antibody. In some embodiments, the human antibody is a monoclonal antibody; thus, in some embodiments, each of the human antibodies in a set can bind to the same epitope on the antigen.

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, (2005) Nat. Biotech. 23: 1117-1125. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, for example, by combining with a different human constant region.

Human antibodies can also be made by hybridoma-based methods. Human myeloma and mouse-human heteromyeloma cell lines for the production of human monoclonal antibodies have been described. (See, for example, Kozbor (1984) J. Immunol, 133: 3001; Brodeur et al., Monoclonal Antibody Production Techniques and Applications, pp. 51-63 (Marcel Dekker, Inc., New York, 1987); and Boerner et al, (1991) J. Immunol., 147:86). Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, (2006) Xiandai Mianyixue, 26(4):265-268 (describing human-human hybridomas). Human hybridoma technology (Trioma technology) is also described in Vollmers and Brandlein, (2005) Histology and Histopathology, 20(3):927-937 (2005) and Vollmers and Brandlein, (2005) Methods and Findings in Experimental and Clinical Pharmacology, 27(3): 185-191.

Human antibodies can also be generated by isolating Fv clone variable domain sequences selected from human-derived phage display libraries. Such variable domain sequences may then be combined with a desired human constant domain. Techniques for selecting human antibodies from antibody libraries are described below.

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, for example, in Hoogenboom et al. in Methods in Molecular Biology 178: 1-37 (O'Brien et al., ed., Human Press, Totowa, N. J., 2001) and further described, for example, in the McCafferty et al, (1990) Nature 348:552-554; Clackson et al, (1991) Nature 352: 624-628; Marks et al, (1992) J. Mol. Biol 222: 581-597; Marks and Bradbury, in Methods in Molecular Biology 248: 161-175 (Lo, ed., Human Press, Totowa, N. J., 2003); Sidhu et al, (2004) J. Mol. Biol. 338(2): 299-310; Lee et al., (2004) J. Mol. Biol. 340(5): 1073-1093; Fellouse, (2004) Proc. Natl. Acad. Sci. USA 101(34): 12467-12472; and Lee et al, (2004) J. Immunol. Methods 284(1-2): 119-132 and PCT publication WO 99/10494.

In certain phage display methods, repertoires of V_{H} and V_{L} genes are separately cloned by polymerase chain reaction (PCR) and recombined randomly in phage libraries, which can then be screened for antigen-binding phage as described in Winter et al., (1994) Ann. Rev. Immunol., 12:433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (for example, from human) to provide a single source of antibodies to a wide range of non-self and also self-antigens without any immunization as described by Griffiths et al., (1993) EMBO J 12:725-734. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter (1992), J. Mol. Biol, 227:381-388. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

In some embodiments, the antibody that inhibits the interaction of TIM-3 and LILRB2 comprises one or more human constant regions. In some embodiments, the human heavy chain constant region is of an isotype selected from IgA, IgG, and IgD. In some embodiments, the human light chain constant region is of an isotype selected from κ and λ. In some embodiments, a human antibody described herein comprises a human IgG constant region. In some embodiments, a human antibody described herein comprises a human IgG4 heavy chain constant region. In some embodiments, a human antibody described herein comprises a human IgG4 constant region and a human κ light chain.

In some embodiments, when effector function is desirable, a human antibody comprising a human IgG1 heavy chain constant region or a human IgG3 heavy chain constant region is selected. In some embodiments, when effector function is not desirable, a human TIM-3 antibody comprising a human IgG4 or IgG2 heavy chain constant region is selected.

As noted herein, the term “human antibody” denotes the genus of possible sequences for the antibody construct, rather than a source of the antibody.

In some embodiments, the antibodies inhibit and/or reduce a tumor intrinsic signal. In some embodiments, the tumor intrinsic signal is one or more signals selected from: a pro-survival signal; an autocrine or paracrine growth signal; a differentiation signal; a STAT-, JAK-, AKT- or PI3K-mediated signal; an anti-apoptotic signal; and a signal promoting and/or necessary for one or more of: tumor invasiveness, metastasis, epithelial-mesenchymal transition, and/or spreading from one tissue or organ to another non-adjacent tissue or organ.

In some embodiments, the antibodies inhibit or reduce immune modulation or immune tolerance to tumor cells. In some embodiments, the antibody inhibits or reduces the activity or activation of one or more cells including, but not limited to: regulatory T-cells (Tregs); myeloid suppressor cells; tumor associated neutrophils (TANs) and tumor associated macrophages (TAMs).

In some embodiments, the antibodies described herein enhance, restore, promote and/or stimulate immune modulation. In some embodiments, the antibodies enhance, restore, promote and/or stimulate the activity or activation of one or more immune cells against tumor cells including, but not limited to: T-cells, cytotoxic T lymphocytes, T helper cells, natural killer (NK) cells, natural killer T (NKT) cells, anti-tumor macrophages (e.g., M1 macrophages), macrophages, B-cells, and dendritic cells.

In some embodiments, the antibodies enhance, restore, promote and/or stimulate the activity and/or activation of T-cells, including, by way of a non-limiting example, activating, enhancing, restoring, and/or stimulation one or more T-cell intrinsic signals, including a pro-survival signal; an autocrine or paracrine growth signal; a proliferative signal; a differentiation signal; a T-cell maturation signal; a p38 MAPK-, ERK-, STAT-, JAK-, AKT- or PI3K-mediated signal; an anti-apoptotic signal; and/or a signal promoting and/or necessary for one or more of: cell survival, cell-cycle progression, T-cell proliferation, glucose metabolism, proteins synthesis and cytokine production.

### Exemplary Antibody Constant Regions

In some embodiments, an antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region.

Throughout the present specification and claims unless explicitly stated or known to one skilled in the art, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

As noted above, whether or not effector function is desirable may depend on the particular method of treatment intended for an antibody. Thus, in some embodiments, when effector function is desirable, the antibody that inhibits the interaction of TIM-3 and LILRB2 comprising a human IgG1 heavy chain constant region or a human IgG3 heavy chain constant region is selected. In some embodiments, when effector function is not desirable, a TIM-3 antibody comprising a human IgG4 or IgG2 heavy chain constant region is selected.

In some embodiments, an antibody comprises a variant Fc region has at least one amino acid substitution compared to the Fc region of a wild-type IgG or a wild-type antibody. In some embodiments, the variant Fc region has two or more amino acid substitutions in the Fc region of the wild-type antibody. In some embodiments, the variant Fc region has three or more amino acid substitutions in the Fc region of the wild-type antibody. In some embodiments, the variant Fc region has at least one, two or three or more Fc region amino acid substitutions described herein. In some embodiments, the variant Fc region herein will possess at least about 80% homology with a native sequence Fc region and/or with an Fc region of a parent polypeptide. In some embodiments, the variant Fc region herein will possess at least about 90% homology with a native sequence Fc region and/or with an Fc region of a parent polypeptide. In some embodiments, the variant Fc region herein will possess at least about 95% homology with a native sequence Fc region and/or with an Fc region of a parent polypeptide.

In some embodiments, an antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, for example, Wright et al. TIBTECH 15:26-32 (1997). The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (for example, complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, that is, between positions 294 and 300, due to minor sequence variations in antibodies. Such fucosylation variants may have improved ADCC function. See, for example, US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, for example, Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, for example, in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, for example, in WO 1997/30087 (Patel et al.); WO 1998/58964 (Raju, S.); and WO 1999/22764 (Raju, S.).

Antibody variants are also provided with amino-terminal leader extensions. For example, one or more amino acid residues of the amino-terminal leader sequence are present at the amino-terminus of any one or more heavy or light chains of an antibody. An exemplary amino-terminal leader extension comprises or consists of three amino acid residues, VHS, present on one or both light chains of an antibody variant.

The in vivo or serum half-life of human FcRn high affinity binding polypeptides can be assayed, for example, in transgenic mice, in humans, or in non-human primates to which the polypeptides with a variant Fc region are administered. See also, for example, Petkova et al. International Immunology 18(12):1759-1769 (2006).

In some embodiments, the antibody variant mediates ADCC in the presence of human effector cells more effectively than a parent antibody. In some embodiments, the antibody variant is substantially more effective at mediating ADCC in vitro, when the amounts of polypeptide variant and parent antibody used in the assay are essentially the same. In some embodiments, the antibody variant is substantially more effective at mediating ADCC in vivo, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

### Antibody Expression and Production

Nucleic acid molecules comprising polynucleotides can encode one or more chains of antibodies that inhibit the interaction of TIM-3 and LILRB2. In some embodiments, a nucleic acid molecule comprises a polynucleotide that encodes a heavy chain or a light chain of an antibody. In some embodiments, a nucleic acid molecule comprises both a polynucleotide that encodes a heavy chain and a polynucleotide that encodes a light chain, of an antibody. In some embodiments, a first nucleic acid molecule comprises a first polynucleotide that encodes a heavy chain and a second nucleic acid molecule comprises a second polynucleotide that encodes a light chain.

In some embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody that inhibits the interaction of TIM-3 and LILRB2 comprises a nucleotide sequence that encodes at least one CDR. In some embodiments, a polynucleotide encoding a heavy chain or light chain of an antibody comprises a nucleotide sequence that encodes at least 3 CDRs. In some embodiments, a polynucleotide encoding a heavy chain or light chain of an antibody comprises a nucleotide sequence that encodes at least 6 CDRs. In some embodiments, a polynucleotide encoding a heavy chain or light chain of an antibody comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

### Vectors

Vectors comprising polynucleotides that encode heavy chains and/or light chains of an antibody that inhibits the interaction of TIM-3 and LILRB2 are provided. Vectors comprising polynucleotides that encode heavy chains and/or light chains are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, for example, in Running Deer et al., Biotechnol. Prog. 20:880-889 (2004).

Antibodies can be screened to determine, for example, their affinity and specificity of binding to TIM-3 or LILRB2, TIM-3 or LILRB2 isoforms, tumor-specific TIM-3 or LILRB2 polypeptides, post-translationally modified TIM-3 or LILRB2 polypeptides, and/or differentially expressed, glycosylated, post-translationally modified and/or spliced TIM-3 or LILRB2 polypeptides by using assays known in the art. For example, the assays may include competitive and noncompetitive assays. Assays of interest include enzyme-linked immunosorbent assay (ELISA), radioimmunoassay (RIA), flow cytometry, etc. Binding assays including Biacore or Octet may also be used. For example, binding assays may use purified or semi-purified TIM-3, or alternatively may use cells that express TIM-3, e.g., cells transfected with an expression construct for TIM-3; T-cells that have been stimulated through cross-linking of CD3 and CD28; the addition of irradiated allogeneic cells, etc. As an example of a binding assay, purified TIM-3 may be bound to an insoluble support, e.g., a microtiter plate, magnetic beads, etc. A candidate agent and soluble, labeled TIM-3 ligand are added to the cells, and the unbound components are then washed off. The ability of the candidate agent to compete with the natural ligand for TIM-3 binding may be determined by quantification of bound, labeled ligand.

In some embodiments, the assay of interest is directed to antibodies that block the binding of TIM-3 to its receptor. In some embodiments, TIM-3 receptor is LILRB2. The antibody will be substantially unreactive with related molecules to TIM-3, such as CD28, other B7 superfamily members, and/or other members of the immunoglobulin superfamily. Further, the antibody does not activate TIM-3 signaling. In another embodiment, the antibody, does not activate TIM-3 signaling but, in some embodiments, may also bind to one or more other members of the B7 superfamily, including B7.1, B7.2, ICOS Ligand, PD-L1, PD-L2, B7-H3, B7-H5, B7-H6 and/or B7-H7. In an exemplary embodiment, a functional assay detects that an agent blocks the binding of TIM-3 to its ligand, for example, by measuring CD4⁺ T-cell proliferation and/or cell cycle progression, release of IL-12, IL-4, IFN-gamma, TNF-alpha, or other cytokines, expression of CD25 and CD69, or the production/emission of a reporter expressed in a cell line engineered to change the production/emission of the reporter when TIM-3 does not bind its receptor, etc.

One skilled in the art may measure changes in cell surface marker expression of TIM-3 or LILRB2 or cellular changes following TIM-3 or LILRB2 activation/inhibition (including, for example, cell cycle progression, and/or cytokine release) using assays that are well known in the art. These assays include, but are not limited to, flow cytometry (including, for example, fluorescent activating cell sorting (FACS)), indirect immune-fluorescence, solid phase enzyme-linked immunosorbent assay (ELISA), ELISpot assays, western blotting (including in cell western), immunofluorescent staining, microengraving (see Han Q et al. Lab Chip. 2010; 10(11):1391-1400), Quant-iT and Qubit protein assay kits, NanoOrange protein quantitation kit, CBQCA protein quantitation kits, EZQ protein quantitation kit, Click-iT reagents, Pro-Q Diamond phosphoprotein stain, Pro-Q glycoprotein stain kits, peptide and protein sequencing, N-terminal amino acid analysis (LifeScience Technologies, Grand Island, N.Y.), chemiluminescence or colorimetric based ELISA cytokine Arrays (Signosis) Intracellular Cytokine Staining (ICS), BD Phosflow™ and BD™ Cytometric Bead Arrays (BD Sciences, San Jose, Calif.); RT-PCR (RT2 Profiler™ Human Common Cytokine PCR Arrays (Cat # PAHS-021) ((SABiosciences/QIAGEN)); CyTOF Mass Cytometer (DVS Sciences, Sunnyvale Calif.); Mass Spectrometry, Microplate capture and detection assay (Thermo Scientific, Rockland, Ill.), Multiplex Technologies (for example Luminex, Austin, Tex.); FlowCellect™ T-cell Activation Kit (EMD Millipore); Surface Plasmon Resonance (SPR)-based technologies (for example Biacore, GE Healthcare Life Sciences, Uppsala, Sweden); CD4⁺ Effector Memory T-cell Isolation Kit and CD8⁺CD45RA⁺ Effector T-cell Isolation Kit (Miltenyi Biotec Inc., CA); The EasySep™ Human T-cell Enrichment Kit (StemCells, Inc., Vancouver, Canada); HumanTh1/Th2/Th17 Phenotyping Kit (BD Biosciences, CA); immunofluorescent staining of incorporated bromodeoxyuridine (BrdU) or 7-aminoactinomycin D. See also, Current Protocols in Immunology (2004) sections 3.12.1-3.12.20 by John Wiley & Sons, Inc., or Current Protocols in Immunology (2013) or by John Wiley & Sons, Inc., the contents of which are herein incorporated by reference in their entirety.

### Host Cells

In some embodiments, heavy chains and/or light chains of an antibody that inhibits the interaction of TIM-3 and LILRB2 may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, TIM-3 heavy chains and/or TIM-3 light chains may be expressed in yeast. See, for example, U.S. Publication No. US 2006/0270045 A1. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the polynucleotides or vectors described herein are also provided. In some embodiments, a host cell comprising an antibody that inhibits the interaction of TIM-3 and LILRB2 is provided. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Antibodies of the invention can be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify a TIM-3 antibody. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the antibody is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, antibodies prepared by the methods described above are provided. In some embodiments, the antibody is prepared in a host cell. In some embodiments, the antibody is prepared in a cell-free system. In some embodiments, the antibody is purified. In some embodiments, the antibody prepared in a host cell or a cell-free system is a chimeric antibody. In some embodiments, the antibody prepared in a host cell or a cell-free system is a humanized antibody. In some embodiments, the antibody prepared in a host cell or a cell-free system is a human antibody. In some embodiments, a cell culture media comprising an antibody is provided. In some embodiments, a host cell culture fluid comprising an antibody is provided.

In some embodiments, compositions comprising antibodies prepared by the methods described above are provided. In some embodiments, the composition comprises an antibody prepared in a host cell. In some embodiments, the composition comprises an antibody prepared in a cell-free system. In some embodiments, the composition comprises a purified antibody. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

In some embodiments, a composition comprising an antibody that inhibits the interaction of TIM-3 and LILRB2 at a concentration of more than about any one of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL is provided. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

In some embodiments, the antibody selectively binds to TIM-3. In some embodiments, the TIM-3 antibody is a monoclonal human antibody. In some embodiments, the TIM-3 monoclonal human antibody has a K_{d} of no larger than 10⁻⁷ for TIM-3, for example, the numerical value is less than 10⁻⁸, 10⁻⁹, 10⁻¹⁰, 10⁻¹¹, 10⁻¹², or lower. In some embodiments, the TIM-3 antibody inhibits or reduces immune modulation or tolerance to tumor cells. In some embodiments, the TIM-3 antibody inhibits or reduces immune modulation or tolerance to tumor cells by inhibiting or reducing the activity or activation of one or more cells selected from: regulatory T-cells (Tregs); myeloid suppressor cells; tumor associated neutrophils (TANs) and tumor associated macrophages (TAMs). In some embodiments, the TIM-3 antibody enhances or restores the activity or activation of T-cells against tumor cells. In some embodiments, the TIM-3 antibody enhances or restores the activity or activation of one or more cells selected from: T-cells, T helper cells, cytotoxic T-cells, dendritic cells, natural killer (NK) cells, natural killer T (NKT) cells, macrophages, anti-tumor macrophages and B-cells. In some embodiments, the TIM-3 antibody enhances or restores a T-cell intrinsic signal.

In some embodiments, TIM-3 activity in the subject is reduced to a level adequate for a therapeutic treatment of the cancer in the subject. In some embodiments, the TIM-3 antibody blocks TIM-3 activity by at least 10%, for example, 20, 30, 40, 50, 60, 70, 80, 90, 95, 99, or 100% blockade of TIM-3 activity.

In some embodiments, the antibody selectively binds to LILRB2. In some embodiments, the LILRB2 antibody is a monoclonal human antibody. In some embodiments, the LILRB2 monoclonal human antibody has a K_{d} of no larger than 10⁻⁷ for LILRB2, for example, the numerical value is less than 10⁻⁸, 10⁻⁹, 10⁻¹⁰, 10⁻¹¹, 10⁻¹², or lower. In some embodiments, the LILRB2 antibody inhibits or reduces immune modulation or tolerance to tumor cells.

In some embodiments, LILRB2 activity in the subject is reduced to a level adequate for a therapeutic treatment of the cancer in the subject. In some embodiments, the LILRB2 antibody blocks LILRB2 activity by at least 10%, for example, 20, 30, 40, 50, 60, 70, 80, 90, 95, 99, or 100% blockade of LILRB2 activity.

### Pharmaceutical Compositions

In some embodiments, compositions comprising antibodies that inhibit the interaction of TIM-3 and LILRB2 are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprising antibodies that inhibit the interaction of TIM-3 and LILRB2 is provided. In some embodiments, the pharmaceutical composition comprises a chimeric antibody that inhibits the interaction of TIM-3 and LILRB2. In some embodiments, the pharmaceutical composition comprises a humanized antibody that inhibits the interaction of TIM-3 and LILRB2. In some embodiments, the pharmaceutical composition comprises a human antibody that inhibits the interaction of TIM-3 and LILRB2. In some embodiments, the pharmaceutical composition comprises an antibody that inhibits the interaction of TIM-3 and LILRB2 prepared in a host cell or cell-free system as described herein. In some embodiments, the pharmaceutical composition comprises a pharmaceutically acceptable carrier.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In general, antibodies that inhibit the interaction of TIM-3 and LILRB2 may be administered in an amount in the range of about 10 μg/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose.

In some embodiments, antibodies that inhibit the interaction of TIM-3 and LILRB2 can be administered in vivo by various routes, including, but not limited to, intravenous, intra-arterial, parenteral, intraperitoneal or subcutaneous. The appropriate formulation and route of administration may be selected according to the intended application.

### Combination Therapy

Antibodies that inhibit the interaction of TIM-3 and LILRB2 can be administered alone or with other modes of treatment. They can be provided before, substantially contemporaneous with, or after other modes of treatment, for example, surgery, chemotherapy, radiation therapy, or the administration of a biologic, such as another therapeutic antibody. In some embodiments, an antibody that inhibits the interaction of TIM-3 and LILRB2 is administered in conjunction with another anti-cancer agent.

In some embodiments, the antibody that inhibits the interaction of TIM-3 and LILRB2 is given concurrently with a second therapeutic agent, for example, a PD-1 therapy. Examples of PD-1 therapy include Nivolumab (BMS-936558, MDX-1106, ONO-4538); Pidilizumab (CureTech, CT-011), Lambrolizumab/pembrolizumab (Merck, KEYTRUDA®, MK-3475); durvalumab (Medimmune/AstraZeneca, MEDI-4736); RG7446/MPDL3280A (Genentech/Roche); MSB-0010718C (Merck Serono); AMP-224 (Amplimmune); BMS-936559; AMP-514 (Amplimmune); MDX-1105 (Merck); TSR-042 (Tesaro/AnaptysBio, ANB-011); STI-A1010 (Sorrento Therapeutics); STI-A1110 (Sorrento Therapeutics); and other antibodies that are directed against programmed death-1 (PD-1) or programmed death ligand 1 (PD-L1).

In some embodiments, the two or more therapeutic agents are administered with a time separation of no more than about 60 minutes, such as no more than about any of 30, 15, 10, 5, or 1 minutes. In some embodiments, the antibody is administered sequentially with a second therapeutic agent. For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

In some embodiments, the antibody is administered with a second therapeutic method for treatment. Thus, the administration of an antibody can be in combination with another system of treatment.

In some embodiments, histological samples of tumors are graded using the antibody described herein according to Elston & Ellis, Histopathology, 1991, 19:403-10, which is hereby incorporated by reference in its entirety. In some embodiments, the antibody described herein is useful in establishing a tumor grade for the purposes of diagnosis or prognosis of a particular cancer.

In some embodiments, the methods described herein are useful for evaluating a subject and/or a specimen from a subject (e.g. a cancer patient). In some embodiments, evaluation is one or more of diagnosis, prognosis, and/or response to treatment.

In some embodiments, the methods described herein comprise evaluating a presence, absence, or level of a protein. In some embodiments, the methods described herein comprise evaluating a presence, absence, or level of expression of a nucleic acid. The compositions described herein may be used for these measurements. For example, in some embodiments, the methods described herein comprise contacting a specimen of the tumor or cells cultured from the tumor with a therapeutic agent as described herein.

In some embodiments, the method can include the measurement of a tumor specimen, including biopsy or surgical specimen samples. In some embodiments, the biopsy is a human biopsy. In various embodiments, the biopsy is any one of a frozen tumor tissue specimen, cultured cells, circulating tumor cells, and a formalin-fixed paraffin-embedded tumor tissue specimen. In some embodiments, the tumor specimen may be a biopsy sample, such as a frozen tumor tissue (cryosection) specimen. As is known in the art, a cryosection may employ a cryostat, which comprises a microtome inside a freezer. The surgical specimen is placed on a metal tissue disc which is then secured in a chuck and frozen rapidly to about −20° C. to about −30° C. The specimen is embedded in a gel-like medium consisting of, for example, polyethylene glycol and polyvinyl alcohol. The frozen tissue is cut frozen with the microtome portion of the cryostat, and the section is optionally picked up on a glass slide and stained. In some embodiments, the tumor specimen may be a biopsy sample, such as cultured cells. These cells may be processed using the usual cell culture techniques that are known in the art. These cells may be circulating tumor cells. In some embodiments, the tumor specimen may be a biopsy sample, such as a formalin-fixed paraffin-embedded (FFPE) tumor tissue specimen. As is known in the art, a biopsy specimen may be placed in a container with formalin (a mixture of water and formaldehyde) or some other fluid to preserve it. The tissue sample may be placed into a mold with hot paraffin wax. The wax cools to form a solid block that protects the tissue. This paraffin wax block with the embedded tissue is placed on a microtome, which cuts very thin slices of the tissue. In certain embodiments, the tumor specimen contains less than about 100 mg of tissue, or in certain embodiments, contains about 50 mg of tissue or less. The tumor specimen (or biopsy) may contain from about 20 mg to about 50 mgs of tissue, such as about 35 mg of tissue. The tissue may be obtained, for example, as one or more (e.g., 1, 2, 3, 4, or 5) needle biopsies (e.g., using a 14-gauge needle or other suitable size). In some embodiments, the biopsy is a fine-needle aspiration in which a long, thin needle is inserted into a suspicious area and a syringe is used to draw out fluid and cells for analysis. In some embodiments, the biopsy is a core needle biopsy in which a large needle with a cutting tip is used during core needle biopsy to draw a column of tissue out of a suspicious area. In some embodiments, the biopsy is a vacuum-assisted biopsy in which a suction device increases the amount of fluid and cells that is extracted through the needle. In some embodiments, the biopsy is an image-guided biopsy in which a needle biopsy is combined with an imaging procedure, such as, for example, X ray, computerized tomography (CT), magnetic resonance imaging (MRI) or ultrasound. In some embodiments, the sample may be obtained via a device such as the MAMMOTOME® biopsy system, which is a laser guided, vacuum-assisted biopsy system for breast biopsy.

In some embodiments, the evaluation may direct treatment (including treatment with the antibodies described herein). In some embodiments, the evaluation may direct the use or withholding of adjuvant therapy after resection. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the antibodies are used as an adjuvant therapy in the treatment of a cancer. In some embodiments, the antibodies are used as the sole adjuvant therapy in the treatment of a cancer. In some embodiments, the antibodies described herein are withheld as an adjuvant therapy in the treatment of a cancer. For example, if a patient is unlikely to respond to an antibody described herein or will have a minimal response, treatment may not be administered in the interest of quality of life and to avoid unnecessary toxicity from ineffective chemotherapies. In such cases, palliative care may be used.

In some embodiments the antibodies are administered as a neoadjuvant therapy prior to resection. In some embodiments, neoadjuvant therapy refers to therapy to shrink and/or downgrade the tumor prior to any surgery. In some embodiments, neoadjuvant therapy means chemotherapy administered to cancer patients prior to surgery. In some embodiments, neoadjuvant therapy means an antibody is administered to cancer patients prior to surgery. Types of cancers for which neoadjuvant chemotherapy is commonly considered include, for example, breast, colorectal, ovarian, cervical, bladder, head and neck, and lung. In some embodiments, the antibodies are used as a neoadjuvant therapy in the treatment of a cancer. In some embodiments, the use is prior to resection.

In some embodiments, the tumor microenvironment contemplated in the methods described herein is one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells; macrophages; neutrophils; and other immune cells located proximal to a tumor.

### Drug Screening

In some embodiments, the invention provides methods for screening an agent for the presence or absence of modulation of the interaction of TIM-3 and LILRB2, the method comprising measuring the binding of TIM-3 and LILRB2 in the presence of a candidate agent, wherein a change in the binding of TIM-3 and LILRB2 in the presence of the candidate agent compared to binding of TIM-3 and LILRB2 in the absence of the candidate agent indicates that the agent modulates the interaction of TIM-3 and LILRB2. In some embodiments, the modulation of the interaction of TIM-3 and LILRB2 is an inhibition of the interaction of TIM-3 and LILRB2. In some embodiments, the inhibition of the interaction of TIM-3 and LILRB2 is an inhibition of the binding of TIM-3 and LILRB2. In some embodiments, the change in binding of TIM-3 and LILRB2 is at least about 1%, 5%, 10%, 20%, 30%, 40%, 50%, 60%, 70%, 80%, 90% or 100%. In some embodiments, the agent that inhibits the interaction of TIM-3 and LILRB2 stimulates the secretion of a monocyte-derived cytokine (e.g., increases the secretion) following administration to an individual.

In some embodiments the TIM-3 and/or the LILRB2 is expressed on a monocyte. In some embodiments the TIM-3 and/or the LILRB2 is expressed on a macrophage. In some embodiments the TIM-3 and/or the LILRB2 is expressed on a dendritic cell.

In some embodiments, the agent is an antibody. In some embodiments, the agent is a small molecule, a peptide, an siRNA polynucleotide antagonists, an RNAi such as siRNA or miRNA, an RNAzymes, a DNAzymes, an oligonucleotide, a nucleotide, or any fragments of these, including DNA or RNA (e.g., mRNA, rRNA, tRNA) of genomic or synthetic origin, which may be single-stranded or double-stranded and may represent a sense or antisense strand, a peptide nucleic acid (PNA), or to any DNA-like or RNA-like material, natural or synthetic in origin, including, e.g., iRNA, ribonucleoproteins (e.g., iRNPs).

In some embodiments, the invention provides methods for screening an agent which inhibits the interaction of TIM-3 and LILRB2, the method comprising measuring the binding of TIM-3 and LILRB2 in the presence of a candidate agent, wherein a reduction in the binding of TIM-3 and LILRB2 in the presence of the candidate agent compared to binding of TIM-3 and LILRB2 in the absence of the candidate agent indicates that the agent inhibits the interaction of TIM-3 and LILRB2. In some embodiments, the inhibition of the interaction of TIM-3 and LILRB2 is an inhibition of the binding of TIM-3 and LILRB2. In some embodiments, the reduction in binding of TIM-3 and LILRB2 is at least about 1%, 5%, 10%, 20%, 30%, 40%, 50%, 60%, 70%, 80%, 90% or 100%. In some embodiments, the agent that inhibits the interaction of TIM-3 and LILRB2 stimulates the secretion (e.g., increases the secretion) of a monocyte-derived cytokine following administration to an individual.

In some embodiments the TIM-3 and/or the LILRB2 is expressed on a monocyte. In some embodiments the TIM-3 and/or the LILRB2 is expressed on a macrophage. In some embodiments the TIM-3 and/or the LILRB2 is expressed on a dendritic cell.

In some embodiments, the agent is an antibody. In some embodiments, the agent is a small molecule, a peptide, an siRNA polynucleotide antagonists, an RNAi such as siRNA or miRNA, an RNAzymes, a DNAzymes, an oligonucleotide, a nucleotide, or any fragments of these, including DNA or RNA (e.g., mRNA, rRNA, tRNA) of genomic or synthetic origin, which may be single-stranded or double-stranded and may represent a sense or antisense strand, a peptide nucleic acid (PNA), or to any DNA-like or RNA-like material, natural or synthetic in origin, including, e.g., iRNA, ribonucleoproteins (e.g., iRNPs).

In an exemplary embodiment, a functional assay that detects T cell activation may be used for confirmation that a candidate agent is an agonist of TIM-3 or activates at least one costimulatory pathway. For example, a population of innate cells expressing TIM-3, for e.g. dendritic cells (DC) may be stimulated with the candidate agent, including an anti-TIM-3 antibody of the invention, in the presence and absence of suboptimal or optimal doses of TLR agonists. An agent that stimulates TIM-3 or activates at least one costimulatory pathway will cause an increase in the production of pro-inflammatory cytokines by DC, which could then lead to T cell activation. T cell activation can be measured by various assays well known in the art. For example, CD4+ T cell proliferation and/or cell cycle progression, release of IL-12 or other cytokines, upregulation of CD25 and CD69, or modulate the production/emission of a reporter expressed in a cell line engineered to change production/emission of the reporter when TIM-3 or at least one costimulatory pathway is activated, etc.

The assay of interest is directed to agents that block the binding of TIM-3 on adaptive immune cells, for example T cells, to its receptor. The agent will be substantially unreactive with related molecules to TIM-3, such as CD28, other B7 superfamily members, and/or other members of the immunoglobulin superfamily. Further, the agent does not activate TIM-3 signaling. In another embodiment, the agent, including antibodies of the invention, does not activate TIM-3 signaling but may also bind to one or more other members of the B7 superfamily, including B7.1, B7.2, ICOS Ligand, PD-L1, PD-L2, B7-H3, B7-H4, B7-H5, B7-H6 and/or B7-H7, or the TIM family, including TIM-1, and/or TIM-4. In an embodiment, this is achieved by the use of monovalent or bivalent binding molecules including bi-specific and/or multispecific antibodies. In an exemplary embodiment, a functional assay detects that an agent blocks the binding of TIM-3 to its ligand, for example, by measuring CD4+ T cell proliferation and/or cell cycle progression, release of IL-12 or other cytokines, expression of CD25 and CD69, or the production/emission of a reporter expressed in a cell line engineered to change the production/emission of the reporter when TIM-3 does not bind its receptor, etc.

The therapeutic agents (e.g. antibodies) described herein inhibit and/or reduce immune modulation and/or immune tolerance to tumor cells. In some embodiments, the therapeutic agent (e.g. antibody) inhibits and/or reduces the activity and/or activation of one or more cells selected from: regulatory T cells (or “Tregs,” which, as used herein, refers to a subpopulation of T cells which modulate the immune system, abrogate autoimmune disease, maintain tolerance to self-antigens and thwart anti-tumor immune responses); myeloid suppressor cells (or “MSC,” which, as used herein, refers to a heterogeneous population of cells, defined by their myeloid origin, immature state, and ability to potently suppress T cell responses); tumor associated neutrophils (or “TANs” which, as used herein, refers to a subset of neutrophils that are found in the tumor microenvironment, capable of supporting tumor growth, and suppressing anti-tumor responses); tumor associated macrophages (or “TAMs” which, as used herein, refers to a subset of macrophages, found in close proximity to a growing tumor mass, and have been shown to have a pro- or anti-tumor role depending on the type of tumor with which they are associated), and/or tumor-inducing mast cells (which as used herein, refers to a subset of bone marrow-derived, long-lived, heterogeneous cellular population).

Exemplary assays to measure the binding of a TIM-3 ligand and/or LILRB2 to TIM-3 by a therapeutic agent (e.g. antibodies, including bispecific and multispecific) described herein are conventional and well known in the art. Exemplary assays include, but are not limited to, ligand binding assay (LBA), including radioimmunoassays (RIA); competitive ligand-binding (CLB) assays; immunohistochemistry, neutralization binding assays, Surface Plasmon Resonance (SPR)-based technologies (for example Biacore, GE Healthcare Life Sciences, Uppsala, Sweden); and fluorescent ligand-binding assays.

The therapeutic agents (e.g. antibodies, including bispecific and multispecific) described herein prevent, inhibit and/or reduce uncommitted/promiscuous preFoxp3 cells (Foxp3+ regulatory (Treg) T cells that transiently express Foxp3, and/or Treg cells that can undergo reprogramming into a phenotype expressing proinflammatory cytokines) from becoming committed FoxP3+ Tregs (a lineage of committed Treg cells that show DNA demethylation of one of the conserved noncoding regions in the FoxP3 gene) called Treg cell-specific demethylation region or TDSR or T-cells. Exemplary assays to measure the prevention, inhibition and/or reduction of FoxP3+ Treg cells, include, but are not limited to, measuring cellular Foxp3 protein expression by western blotting or immunofluorescence; functional assays such as production of anti-inflammatory cytokines such as TGF-β or IL-10; proliferation assays such as incorporation of BrdU or tritiated-thymidine, or CFSE dilution, cell viability assays such as incorporation of 7-aminoactinomycin D, mitochondrial activity or caspase assays, and TUNEL assays, cytolysis or membrane leakage assays using propidium iodide or trypan blue.

The therapeutic agents (e.g., antibodies, including bispecific and multispecific) described herein stimulate, induce and/or increase the presentation and/or cross-presentation of a tumor antigen in professional and/or certain non-professional antigen-presenting cells including innate cells and/or dendritic cells. Exemplary tumor antigens include, but are not limited to, a polypeptide, a carbohydrate, a nucleic acid or a DNA molecule, including, but not limited to Tumor-Specific Antigens (TSA), which are present only on tumor cells and not on any other cell; Tumor-Associated Antigens (TAA), which are present on some tumor cells and also some normal cells; products of oncogenes and tumor suppressor genes; oncofetal antigens; cell type-specific differentiation antigens; alphafetoprotein (AFP); carcinoembryonic antigen (CEA); CA-125; mucins (e.g. MUC-1); epithelial tumor antigen (ETA); melanoma-associated antigen (MAGE) 1, 2, and 3; MART-1/Melan-A; gp100; HER-2; prostate-specific antigen (PSA); prostatic acid phosphatase (PAP); and viral proteins such as hepatitis B (HBV), Epstein-Barr (EBV), and human papilloma (HPV). See, e.g., Abbas, A. K, and Lichtman, 2005. A. H. Cellular and Molecular Immunology. Elsevier Saunders, Philadelphia. Presentation and/or cross presentation of a tumor antigen as used herein denotes the ability of certain professional and/or certain non-professional antigen-presenting cells. (e.g., innate cells and/or B cells) to take up, process and present tumor antigens with MHC class I and/or class II molecules to T cells to stimulate immunity against tumors. Exemplary innate cells include dendritic cells, macrophages, epithelial cells, endothelial cells, natural killer (NK) cells, γδT cells. Exemplary assays to identify and/or measure the stimulation, induction and/or increase in the presentation and/or cross-presentation of a tumor antigen are conventional and well known in the art including, (1) direct staining of antigens using fluorophore-labeled-, radiolabeled-chemical labeled-antigen-specific antibodies of antigen presenting cells, antigen retrieval and identification using mass spectrometry; and/or (2) antigen-specific versus non-specific T cell activation, using functional, proliferation and/or cell viability assays.

The therapeutic agents (e.g. antibodies, including bispecific and multispecific) described herein inhibit, block and/or reduce cell death of anti-tumor CD8+ and/or CD4+ T cells. In some embodiments the therapeutic agents (e.g. antibodies, including bispecific and multispecific) described herein stimulate, induce, and/or increase cell death of pro-tumor T cells. T cell exhaustion is a state of T cell dysfunction characterized by progressive loss of proliferative and effector functions, culminating in clonal deletion (See, e.g., Virgin et al. (2009) Cell 138:30-50). Accordingly, as used herein the term “pro-tumor T cells” refers to T cells that have a loss of proliferative and effector functions and/or have been clonally deleted. In addition, as used herein the term “anti-tumor CD8+ and/or CD4+ T cells” refers to T cells that can mount an immune response to a tumor. Exemplary pro-tumor T cells include, but are not limited to, Tregs, Th2 cells, dysfunctional CD4+ Th1 cells and CD8+ T cells that express high levels of any of the checkpoint inhibitory/exhaustion markers, such as TIM-3, B7-H3, B7-H4, PD-1, and CTLA-4. Assays to identify and measure the cell death of anti-tumor CD8+ and/or CD4+ and/or pro-tumor T cells are conventional and well known in the art. For example, cell viability assays such as mitochondrial activity or caspase assays, and TUNEL assays, cytolysis or membrane leakage assays using propidium iodide or trypan blue, functional assays such as cell motility assays, and genomic and proteomic assays such as DNA microarrays and protein chips to analyze cell stress pathways.

The therapeutic agents (e.g. antibodies, including bispecific and multispecific) described herein reduce and/or deplete TIM-3 expressing cells and/or TIM-3 expressing cells located within the tumor microenvironment. Assays to identify and measure the reduction and/or depletion of TIM-3 expressing cells are conventional and well known in the art. For example, cell viability or cell death assays such as mitochondrial activity or caspase assays, and TUNEL assays, cytolysis or membrane leakage assays using propidium iodide or trypan blue, functional assays such as cell motility assays, and genomic and proteomic assays such as DNA microarrays and protein chips to analyze cell stress pathways.

### Kits

Also provided are articles of manufacture and kits that include any of the antibodies that modulate (e.g., inhibit) the interaction of TIM-3 and LILRB2 as described herein, and suitable packaging. In some embodiments, the invention includes a kit with (i) an antibody that modulates (e.g., inhibits) the interaction of TIM-3 and LILRB2 and (ii) instructions for using the kit to administer the antibody to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable for treatment.

The containers may be unit doses, bulk packages (e.g., multi-dose packages) or sub-unit doses. For example, kits may also be provided that contain sufficient dosages of antibodies disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of antibodies and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of antibody.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Activated Peripheral Blood Mononuclear Cells Respond to Anti-TIM-3 Blockade

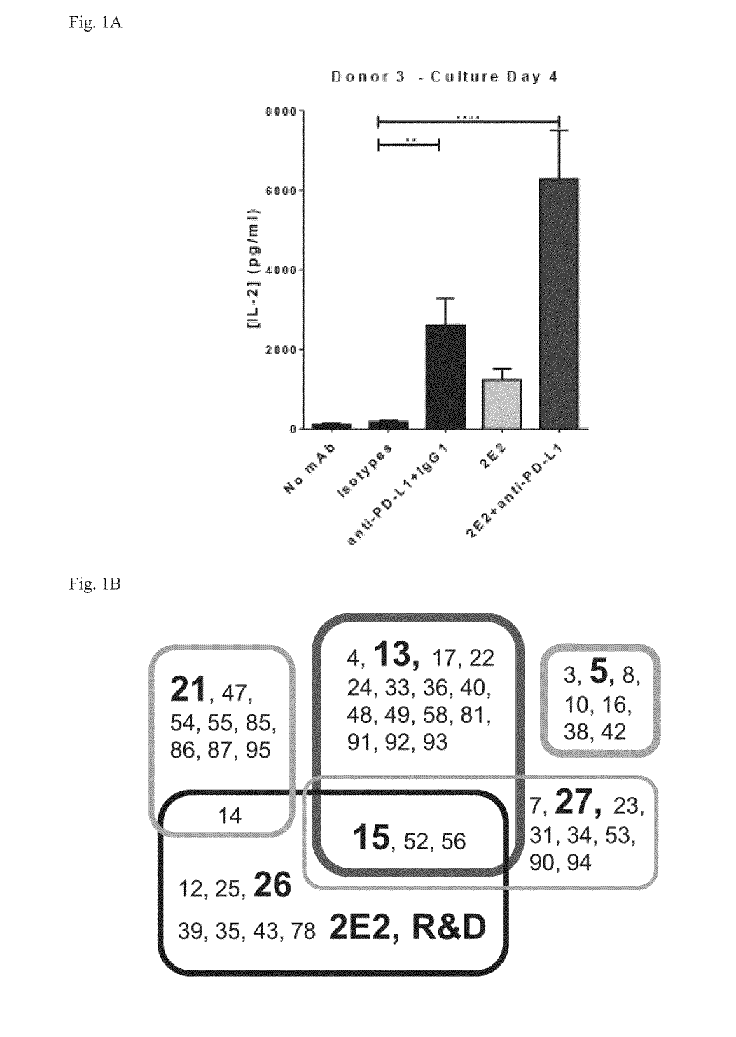

Whole blood samples activated with Staphylococcal enterotoxin B (SEB) (Calbiochem, 324798, Billerica, Mass.) have been shown to respond to an immune checkpoint blockade using an anti-PD-1 antibody as shown by increases of IL-2 secretion (EP2170959B1). The SEB assay was adapted to show that SEB-activated PBMCs demonstrate activity in response to TIM-3 blockade using anti-TIM-3 mAb F38-2E2 (BioLegend, 345010, San Diego, Calif.), either alone or in conjunction with an anti-PD-L1 antibody (Clone 29E.2A3, BioLegend, 329716, San Diego, Calif.) (FIG. 1A). Alone, F38-2E2 addition to the cultures facilitated IL-2 release similar to anti-PD-L1 blockade, but at approximately 50% the activity compared to anti-PD-L1. When employing the combination of TIM-3 and PD-L1 blockade in the assay, a synergistic increase in IL-2 secretion was seen in comparison to either antibody on its own. The increases were >250% and >600% when comparing the combination to anti-PD-L1 alone or F38-2E2 alone, respectively.

Anti-human TIM-3 monoclonal antibodies (mAbs) were generated by immunization of mice and hybridoma fusion techniques. FIG. 1B shows the respective diverse bins for the mAb clones when arranged according to their ability to cross-block one another in binding plate-bound TIM-3 protein.

### Methods

Generation of a Panel of Mouse-Anti-Human TIM-3 Monoclonal Antibodies.

BALB/c or SJL mice were immunized and boosted with 50 μg each of Human and Mouse TIM-3-Fc Protein up to 4 times over 3 months. Splenocytes were fused to mouse myeloma cells and selected in HAT media (containing hypoxanthine, aminopterin, and thymidine). Hybridoma supernatants were screened by ELISA for binding to human and mouse TIM-3-Fc protein. ELISA positive clones were expanded and screened for binding to human TIM-3 overexpressing 293FT cells. Hybridomas that bound human TIM-3 were subcloned by limiting dilution and confirmed by binding to human TIM-3 expressing CHOK1 or 293FT cells by flow cytometry and binding human and TIM-3-Fc by ELISA.

SEB Assay.

FIG. 1A shows TIM-3 blockade enhances T cell cytokine secretion and acts synergistically with PD-L1 blockade. Peripheral blood mononuclear cells (PBMCs) were isolated from blood of fresh donors by Ficoll separation and frozen in 90% fetal bovine serum (FBS), 10% DMSO at −150° C. for long term storage. PBMCs were thawed into complete RPMI media containing 10% FBS, 50 nM 2-Mercaptoethanol, Non-Essential Amino Acids, 1 mM Sodium Pyruvate, 10 mM HEPES. 100,000 cells were plated in each well of a 96-well plate in complete RPMI. Anti-human PD-L1 was added at 10-50 μg/ml and anti-human TIM-3 was added at 50 μg/ml as indicated. Cells and mAbs were incubated at 37° C. for 30 minutes and SEB was added at a final concentration of 1 μg/ml. After 4 days of activation, supernatant was collected and frozen at −20° C. Cytokine concentration was measured using multi-parameter cytokine bead array (Becton, Dickinson and Company, 558270, Franklin Lakes, N.J.). IL-2 was found to be the cytokine most significantly influenced by TIM-3 and PD-L1 blockade from the array measured. Data are representative of at least 4 healthy donors.

Antibody Epitope Bins.

Monoclonal antibodies were compared in pairwise fashion. One mAb was bound to a plate (Nunc, 442404, Rochester, N.Y.) overnight at 4° C. (1 μg/ml). Comparison mAbs were individually combined in excess (10 μg/ml) with biotinylated hTIM-3Fc (10 nM) and incubated at 25° C. for 2 h, then applied to the antibody coated wells of the plate and incubated for another hour at 25° C. Amounts of hTIM-3-Fc captured on the plate were measured in a colorimetric assay using Streptavidin-horseradish peroxidase (HRP) with 3,3′,5,5′-tetramethylbenzidine (TMB) (Sigma-Aldrich, 860336, St. Louis, Mo.) as a substrate. TMB substrate neutralized with H₂SO₄ prior to reading optical density at 450 nm wave length (OD₄₅₀) utilizing a Biotek plate reader (Biotek, Synergy H1, Winooski, Vt.).

### Example 2

### SEB Induction of TIM-3 on Macrophages has Different Kinetics Compared to T Cells

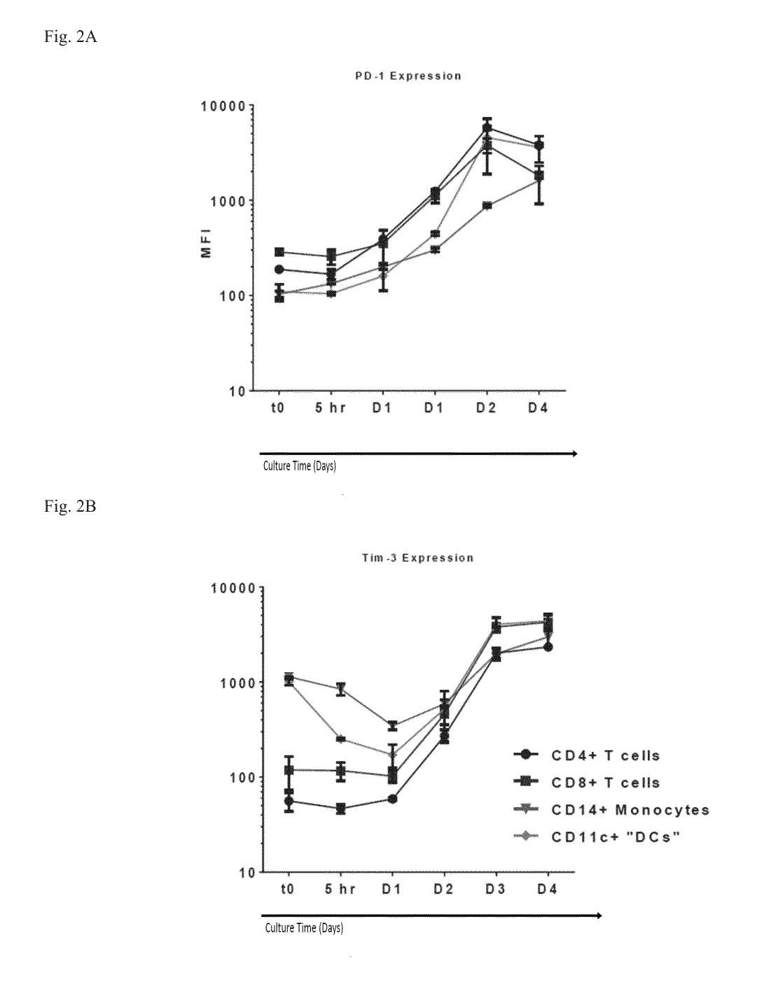

To characterize the SEB-activated PBMC assay more closely, surface expression of PD-1 and TIM-3 proteins were measured over time via flow cytometry while discriminating the most relevant cell types. PD-1 expression was rather uniform among the different cell populations and gradually increased 10-fold over the course of 3 days when it reaches its peak (FIG. 2A). TIM-3 was expressed more diversely at the start of the assay where it was found at a much higher degree in CD14+ monocytes/macrophages and CD11c+ DCs in comparison to T cells (FIG. 2B). Over time, TIM-3 surface expression decreased on monocytes/macrophages and DCs reaching its lowest amounts at 24 hours and then all populations increased surface protein until reaching a pinnacle on day 3. From these data, TIM-3 blockade had a greater impact on monocyte/macrophage and DC biology early on in the assay while potentially influencing all cells directly or indirectly (monocytes, DCs, and T cells) as time passed.

### Methods

Surface expression of TIM-3 and PD-1 was measured during the first 4 days of SEB activation of human PBMCs. 100,000 PBMCs isolated from blood of healthy human donors were plated in each well of a 96 well plate in complete RPMI. SEB was added at a final concentration of 1 μg/ml. At various time points, replicate wells of cells from 2 donors were removed, washed once with PBS containing 2% FBS and 0.05 M sodium azide, stained for 20 minutes on ice with antibodies specific for human CD11c (Clone 3.9, BioLegend, 301608, San Diego, Calif.), CD14 (Clone M5E2) (BioLegend, 301804, San Diego, Calif.), CD8 (Clone RPA-T8) (BioLegend, 301044, San Diego, Calif.), CD4 (Clone RPA-T4) (BioLegend, 300556, San Diego, Calif.), PD-1 (Clone EH12.2H7) (BioLegend, 329906, San Diego, Calif.) and TIM-3 (BioLegend, 345012, San Diego, Calif.). Surface protein expression is expressed as the average mean fluorescence intensities (MFI) of replicate wells from 2 donors.

### Example 3

### SEB Induction of Innate Inflammatory Cytokines

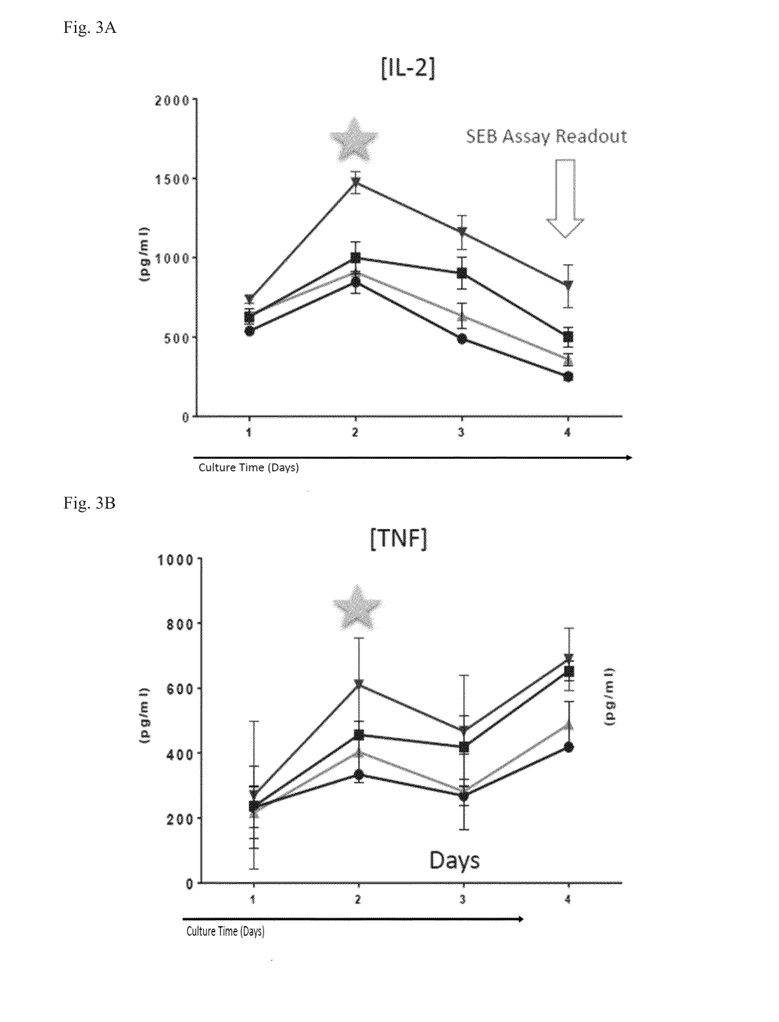

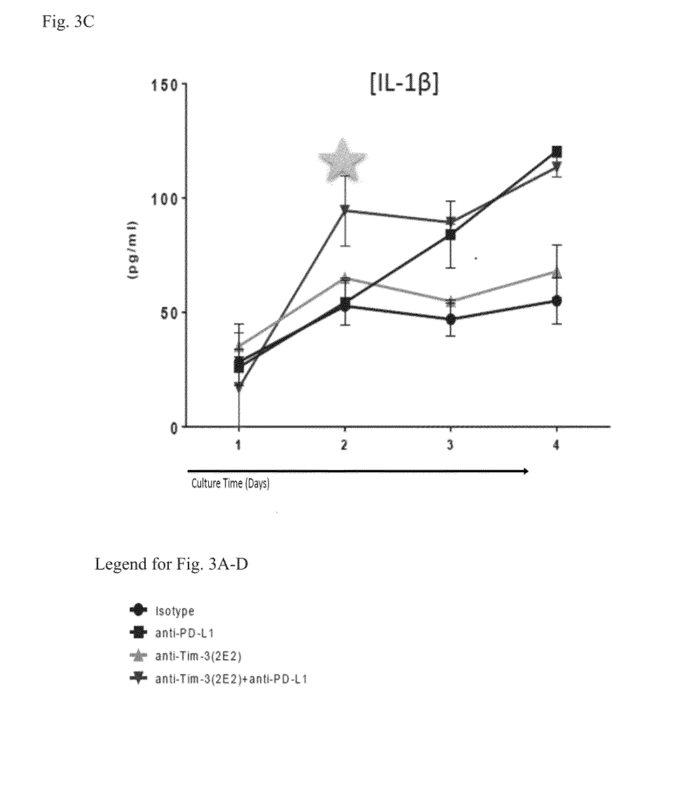

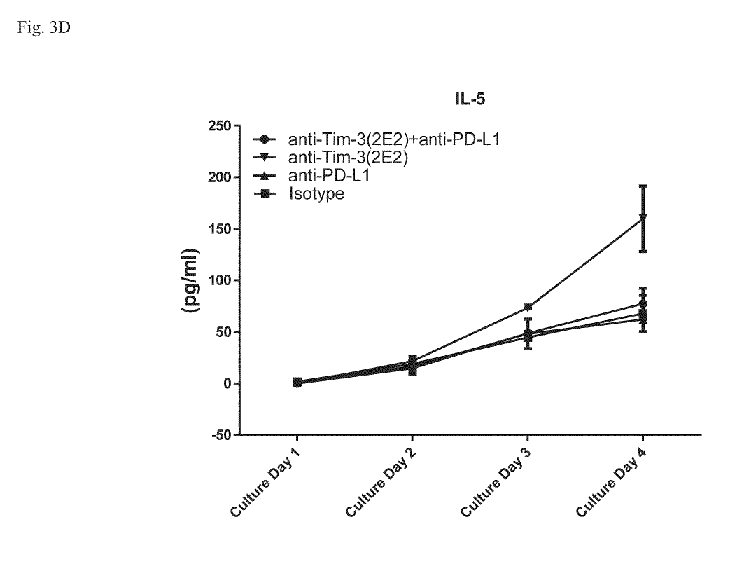

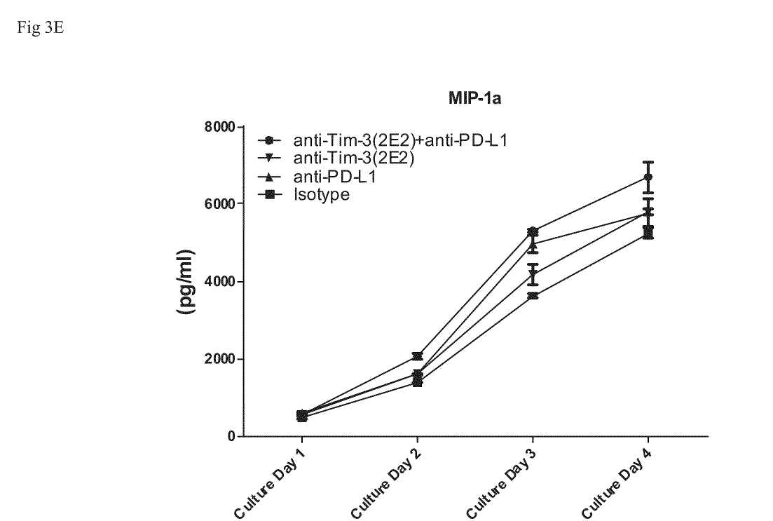

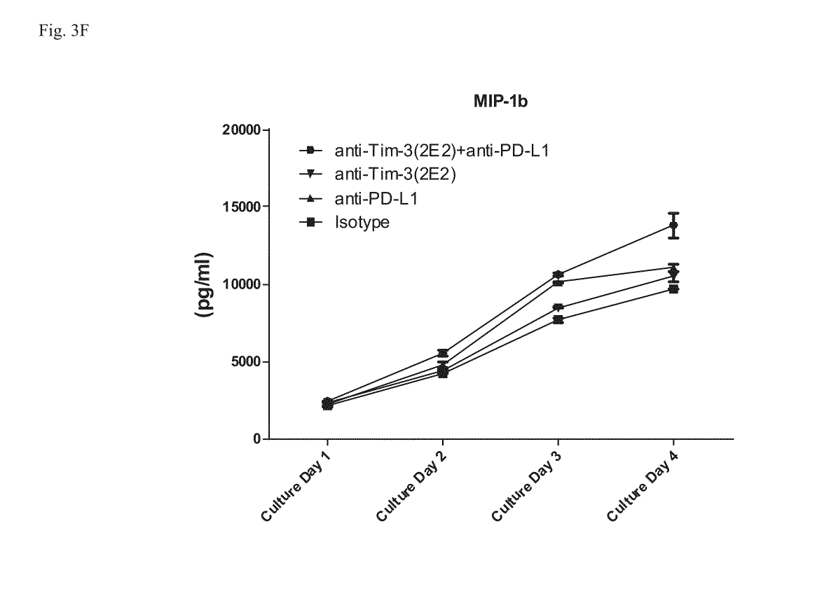

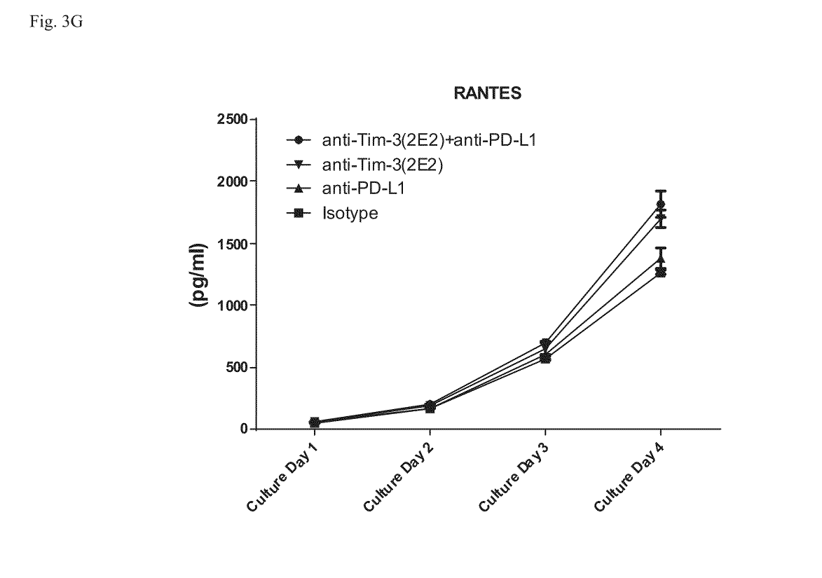

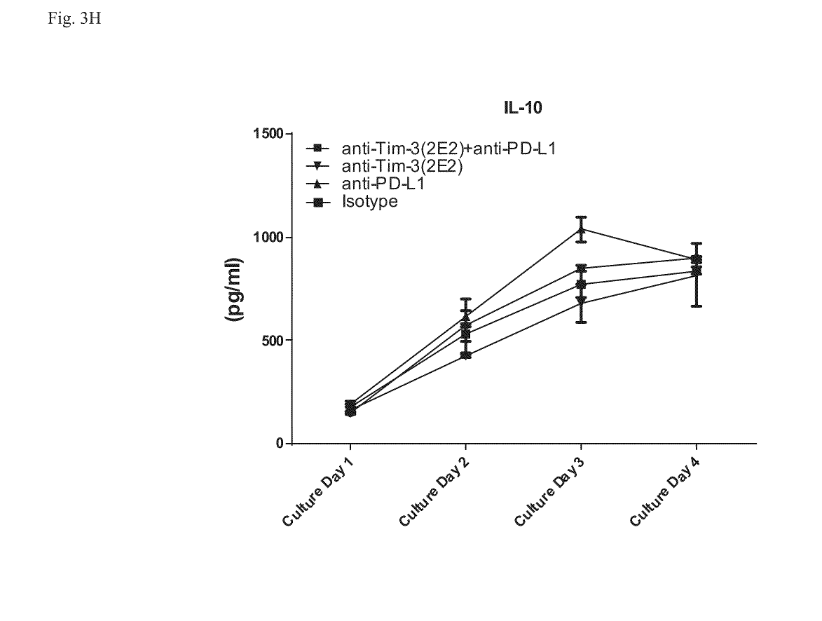

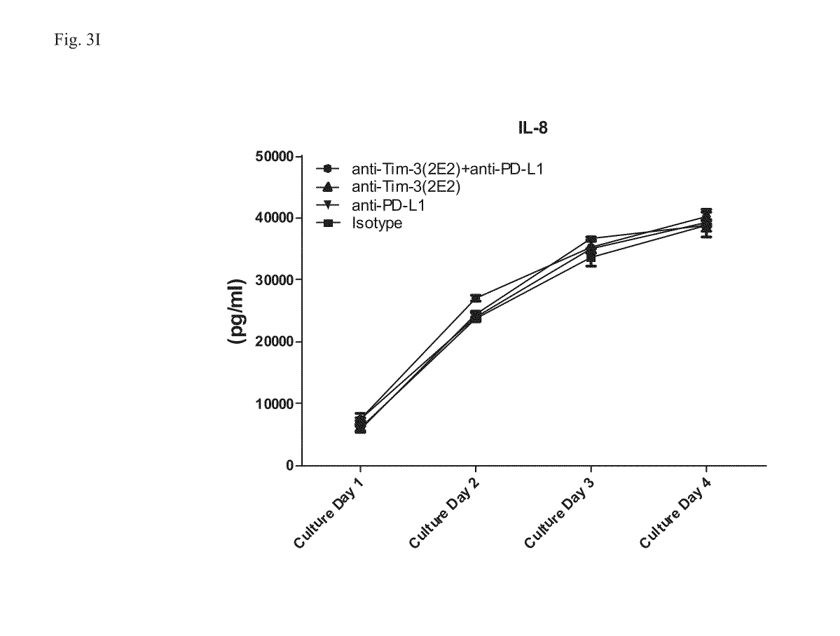

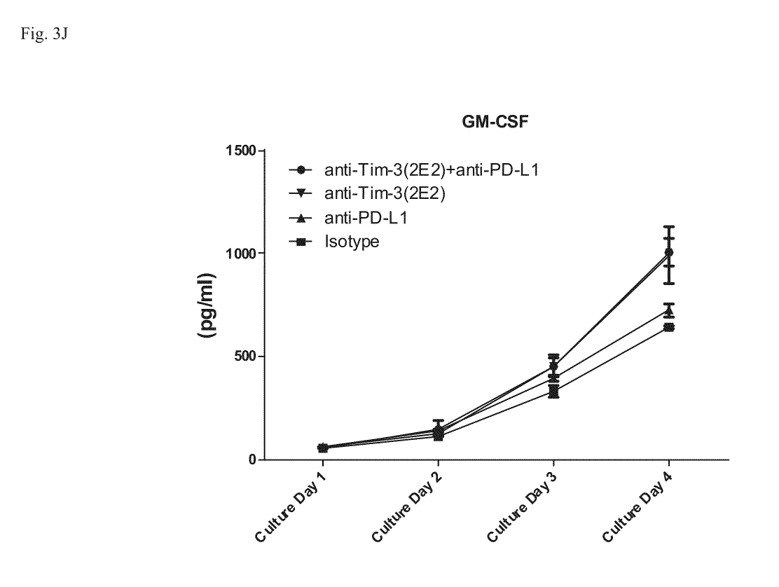

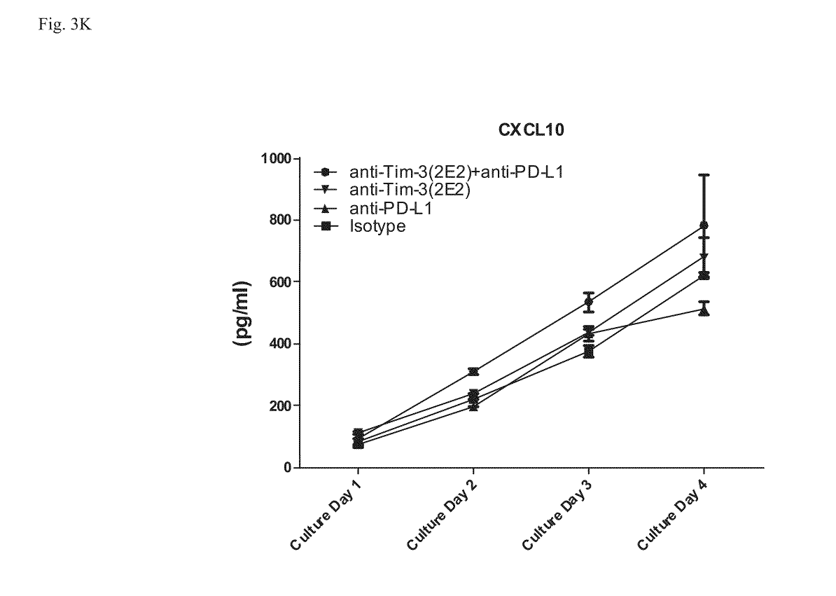

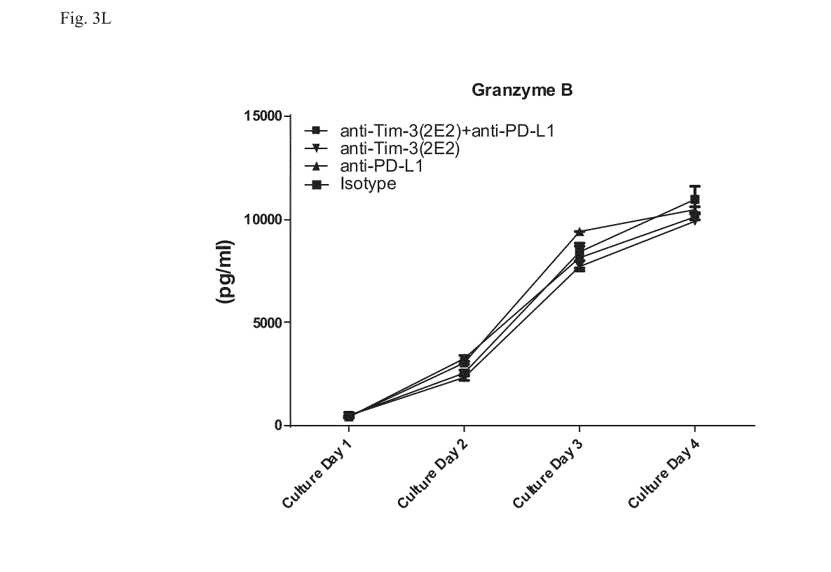

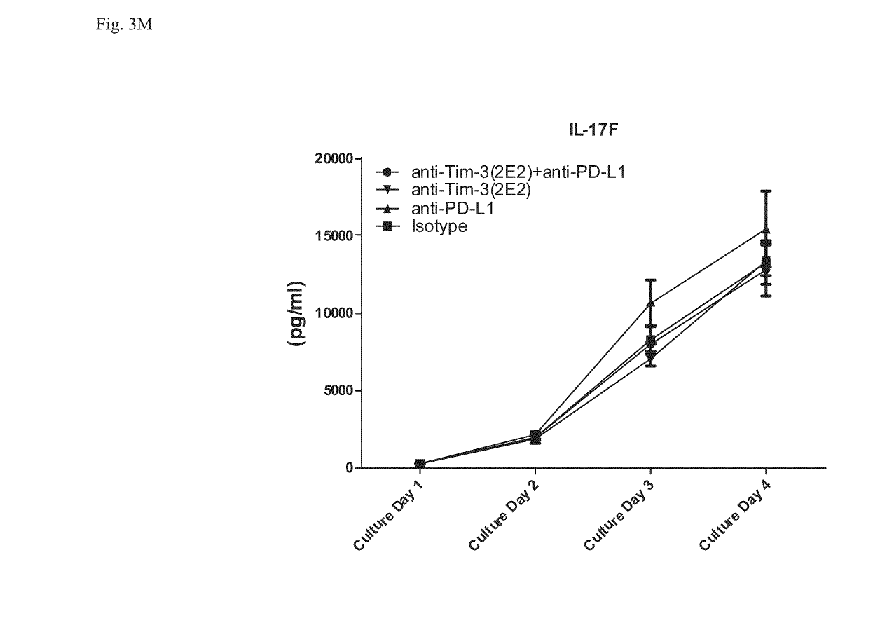

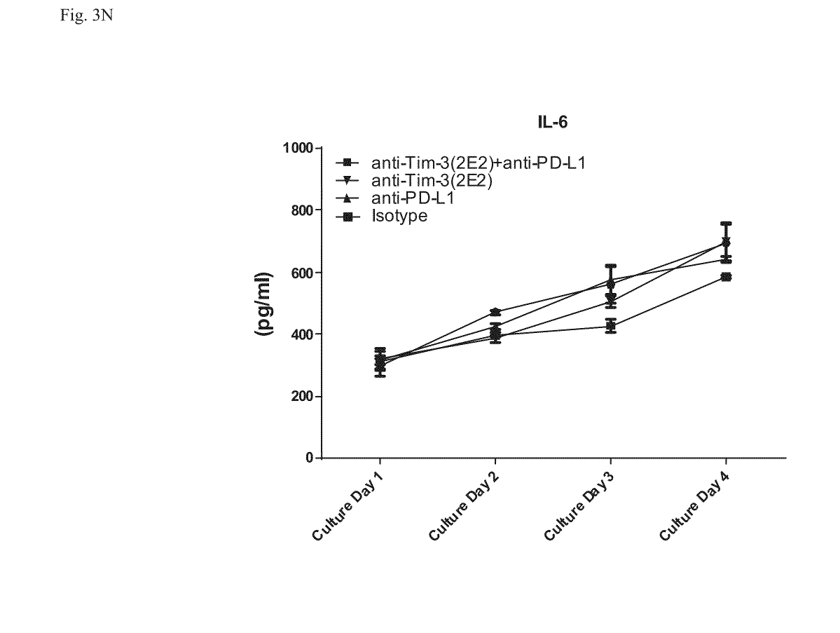

The secretion of several cytokines during the course of SEB activation of PBMC was examined to measure the impact of TIM-3 blockade at the time of the dominant presence of the target on cells of the innate immune system. Increases in TNFα (FIG. 3B) and IL-1β(FIG. 3C) secretion, in addition to IL-2 secretion (FIG. 3A), occurred by day 2. The secretion of other cytokines is shown in FIGS. 3D-3O. These results suggest that TIM-3 blockade leads to activation of the myeloid cells in the assay. These results suggest that these analytes can also serve as readouts for monitoring the effects of anti-TIM-3 mAbs.

### Methods

Cytokine expression was assessed at various time points during SEB activation. 100,000 PBMCs isolated from blood of healthy human donors were plated in each well of a 96 well plate in complete RPMI. Anti-human PD-L1 was added at 10 μg/ml and/or anti-human TIM-3 was added at 50 μg/ml. Cells and mAbs were incubated at 37° C. for 30 minutes and SEB was added at a final concentration of 1 μg/ml. After 1, 2, 3 or 4 days, a sample of supernatant was collected and frozen at −20° C. All samples from each time point were measured for cytokine content using multi-parameter cytokine bead array. Selected cytokines are shown in FIGS. 3A-3C, data are representative of PBMCs from 2 healthy donors.

### Example 4

### TIM-3 is More Strongly Associated with Myeloid Cells than T Cells in Human Cancers

A set of ˜8000 human tumors representing 20 different indications was used for this analysis Immune genes that are known to come from the same cell type show very high levels of correlation. For example, expression of CD3 genes (CD3γ, CD3δ, and CD3ε), that are a part of the TCR complex common to T cells, show very high correlation with the expression of both CD4 and CD8 genes. The expression of genes that indicate state of T cell activation, such as PD-1 and ICOS, also show good correlation.

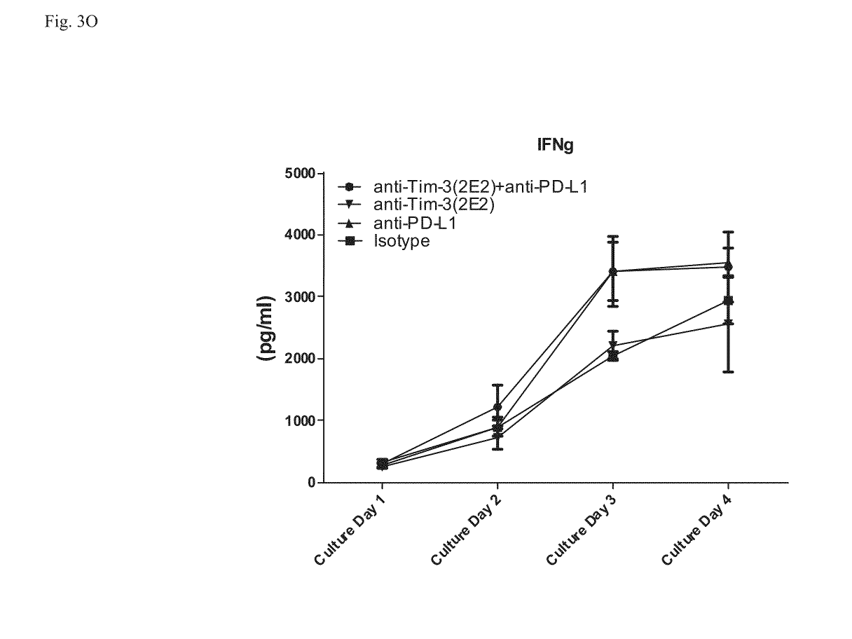

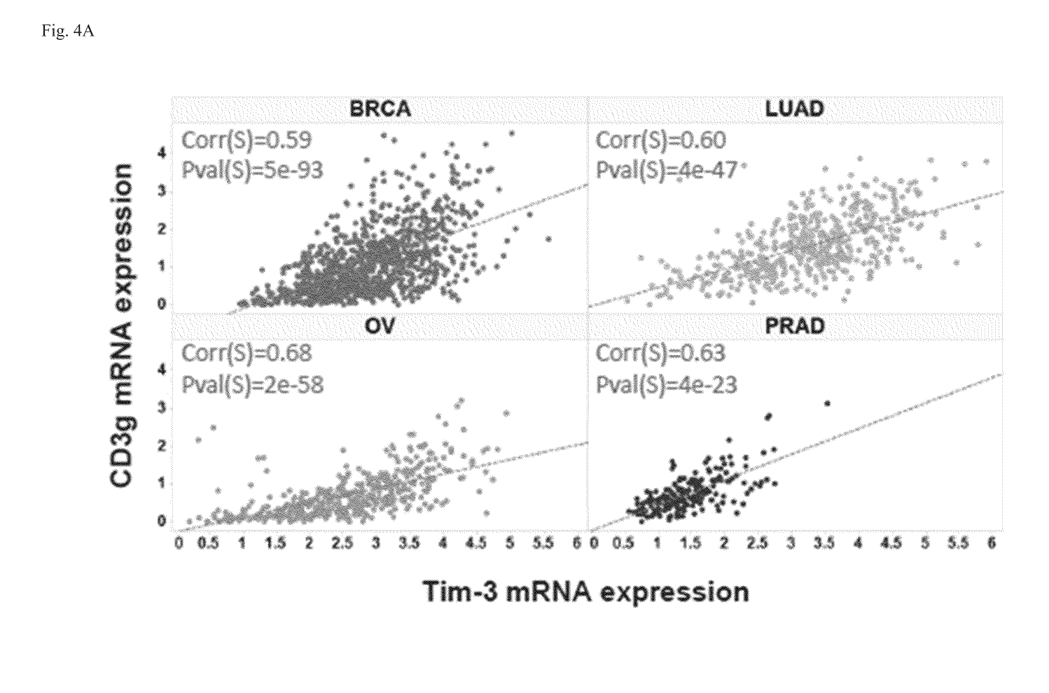

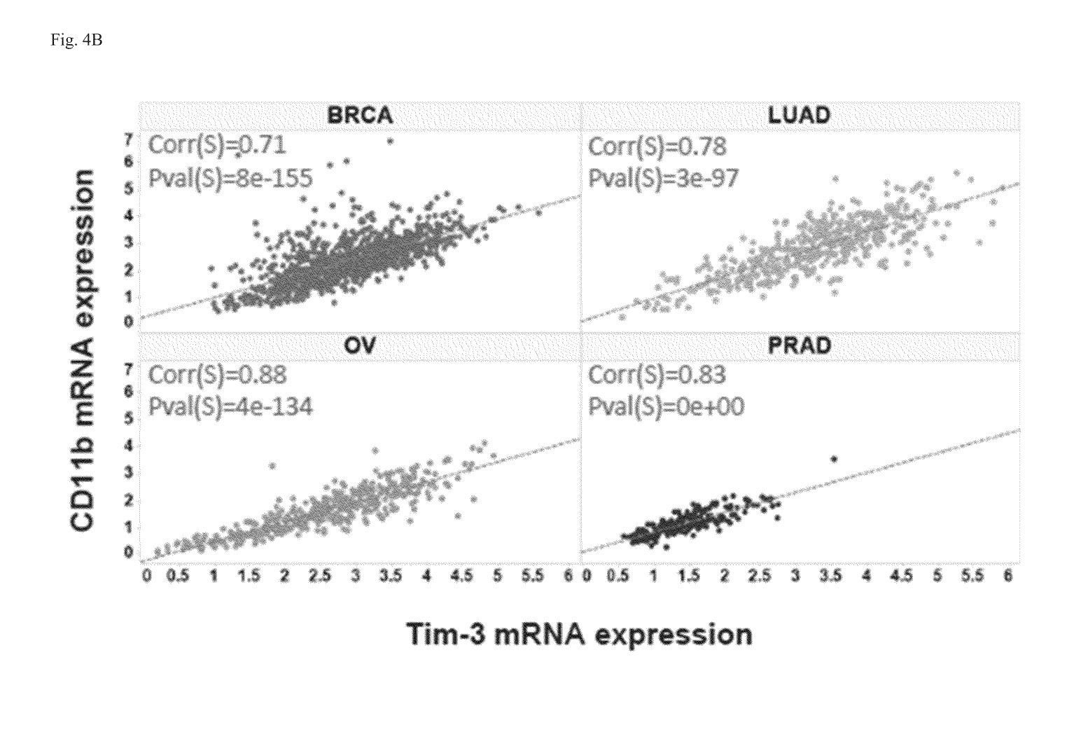

As TIM-3 was believed to function as a T cell function inhibitor, TIM-3 expression was evaluated for correlation with major T cell markers. However, the correlation of TIM-3 expression and T cell markers was poor to average across multiple tumor types (FIG. 4A). Surprisingly, TIM-3 expression showed a very tight correlation with various established myeloid cell markers, such as CD11b or CD11c, across multiple tumor types, including breast cancer, lung cancer, ovarian cancer, prostate cancer, and head and neck cancer (FIG. 4B). The strength of these correlations suggests that TIM-3 is predominantly expressed by, and its function is majorly mediated by, tumor-associated monocyte/macrophages and dendritic cells in the human tumor microenvironment.

### Methods

RNA Sequencing Data from ˜8000 Individual Tumors.

The sequence data was normalized and processed for expression and mutational analysis by specialized software (OmicSoft, Cary, N.C.). TIM-3 transcripts levels were correlated to various immune cell type specific genes across all of the available tumor samples using MatLabR2013b software (Mathworks Inc., Natick, Mass.).

### Example 5

### Activation of Dendritic Cells and Macrophages by Anti-TIM-3 Antibodies

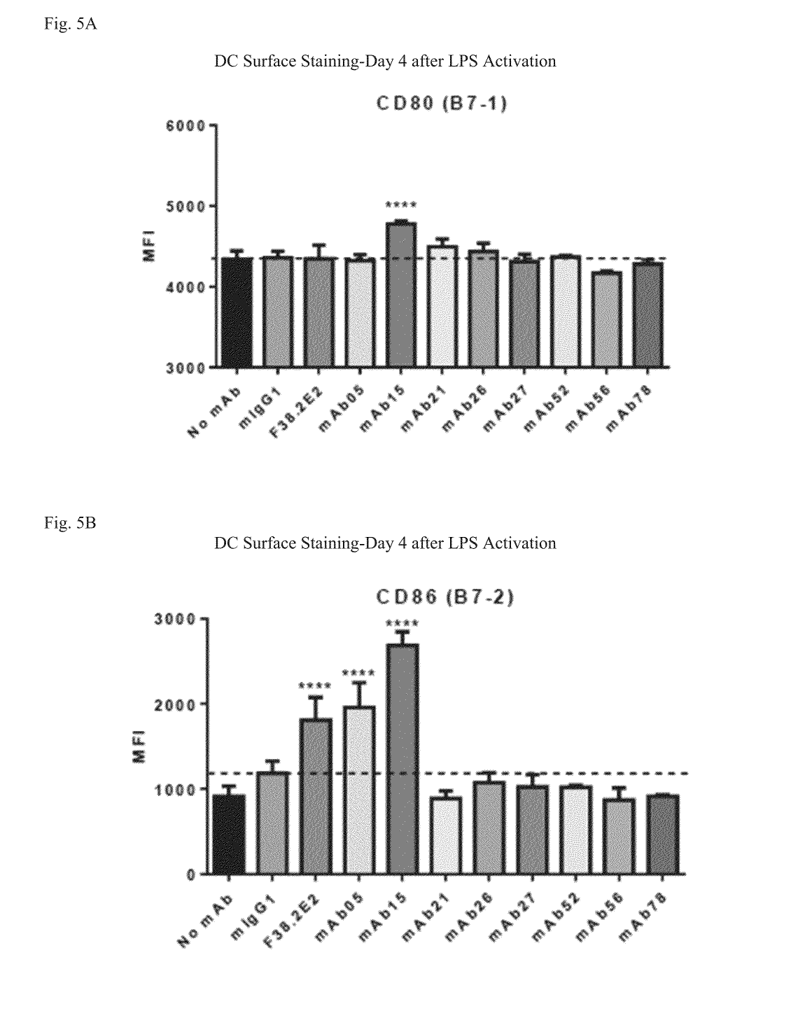

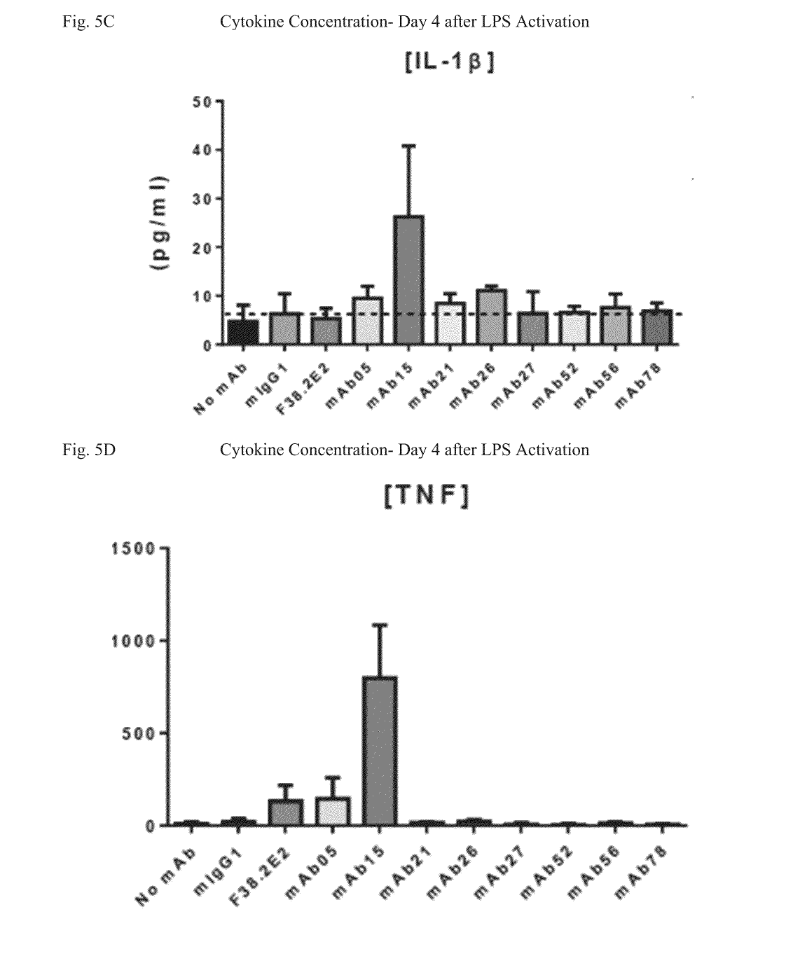

To examine the effects of TIM-3 blockade solely on monocyte/macrophage and DC populations, monoculture systems of activated cells from each cell type were evaluated in isolation. PBMC derived DCs upon stimulation with LPS were activated to a greater degree when cultured with TIM-3 specific mAbs (FIG. 5). TIM-3 blockade led to increases in the expression of costimulatory molecules CD80 (clone 2D10, BioLegend, 305218, San Diego, Calif.) (FIG. 5A) and CD86 (clone IT2.2, BioLegend, 305430, San Diego, Calif.) (FIG. 5B) and the secretion of effector molecules TNFα (Becton, Dickinson and Company, 560112, Franklin Lakes, N.J.) (FIG. 5C), IL-1β(Becton, Dickinson and Company, 558279, Franklin Lakes, N.J.) (FIG. 5D), and IL-12 (Becton, Dickinson and Company, 560154, Franklin Lakes, N.J.) (FIG. 5E). Similar results were found under the same conditions except using LPS-activated peripheral blood derived macrophages. Such changes in these macrophages resemble those of the more inflammatory type population typically referred to as “M1”. These results confirm that TIM-3 mAb blockade can impact macrophage and DC biology.

### Methods

PBMCs were isolated by Ficoll separation from 100 ml of fresh whole blood from two donors. CD14 negative selection (Stemcell Technologies, 19058, Vancouver, BC, Canada) was carried out on all cells from each donor according to manufacturer's protocol. 1 million cells per well added to a 6 well plate in Monocyte Derived DC (MDDC) media (RPMI with 10% FBS, 20 ng/ml rhIL-4 (BioLegend, 574008, San Diego, Calif.), 20 ng/ml rhGM-CSF (BioLegend, 572905, San Diego, Calif.)) or Monocyte Derived Macrophage (MDM) media (RPMI with 10% FBS, 50 ng/ml rhM-CSF (Biolegend, 574804, San Diego, Calif.). Media was changed at culture day 2, 4 and 6. On culture day 8, DCs were harvested, pooled, counted and were assessed by flow cytometry for expression of MHC-II (HLA-DR, CD86 and CD209 (BioLegend, 330110, San Diego, Calif.). At least 80% of cells were positive for CD86, CD209 and HLA-DR. DCs were arrayed in fresh RPMI 10% FBS at 10-20,000 per well in 100 μl in a 96 well round bottom plates as outlined above. 100 μl of RPMI containing 200 ng/ml LPS was added to each well, as indicated. Antibody was added at 50 μg/ml. Antibodies used included anti-TIM-3 antibodies generated as described in Example 1, mAb F38-2E2, and a mouse IgG1 isotype. After 4 days of LPS activation, supernatant was collected and frozen at −20° C., DCs were dissociated from the plate, washed once in PBS with 2% FBS, and stained for surface expression of CD209 (Biolegend Clone 9E9A8), MHC-II (HLA-DR; clone L243, BioLegend, 307644, San Diego, Calif.), CD80,CD86. CD80 and CD86 expression is shown as the MFI for each gated on the CD209+ MHC-II+ events among live cells (FIG. 5F). Supernatant cytokine concentration was measured using multi-parameter cytokine bead array. Selected cytokines are shown in FIG. 5, data are representative of DCs from 6 healthy donors in 3 experiments.

### Example 6

### Human LILRB2 Binds to Human TIM-3

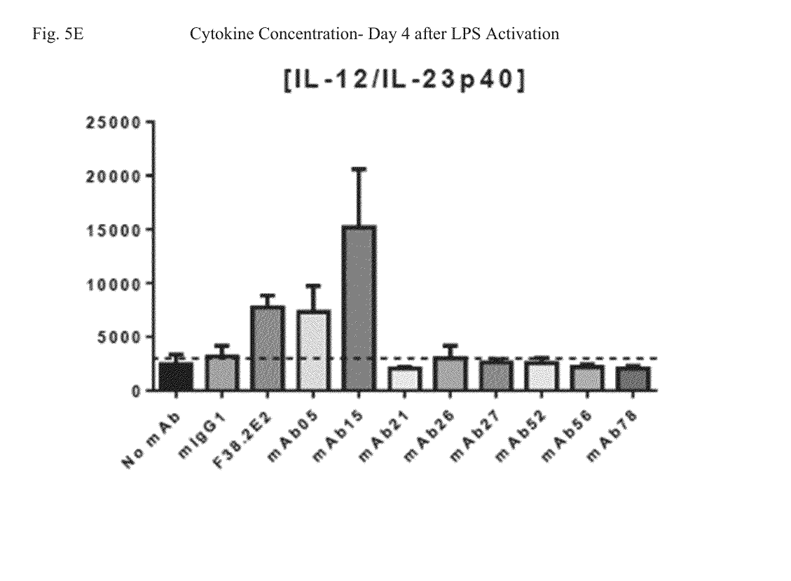

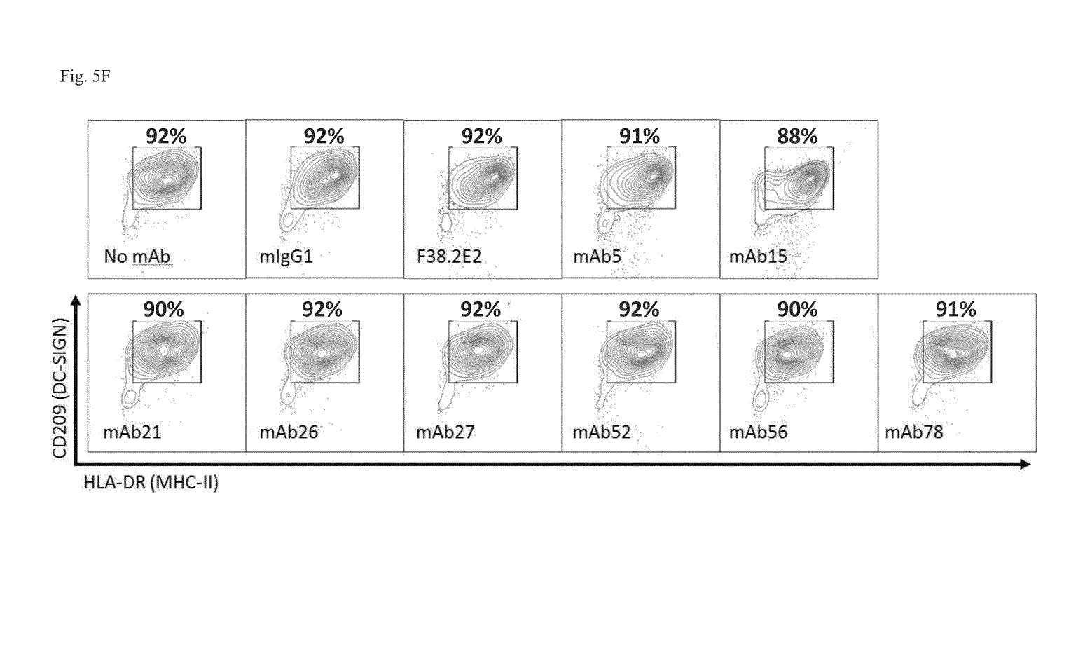

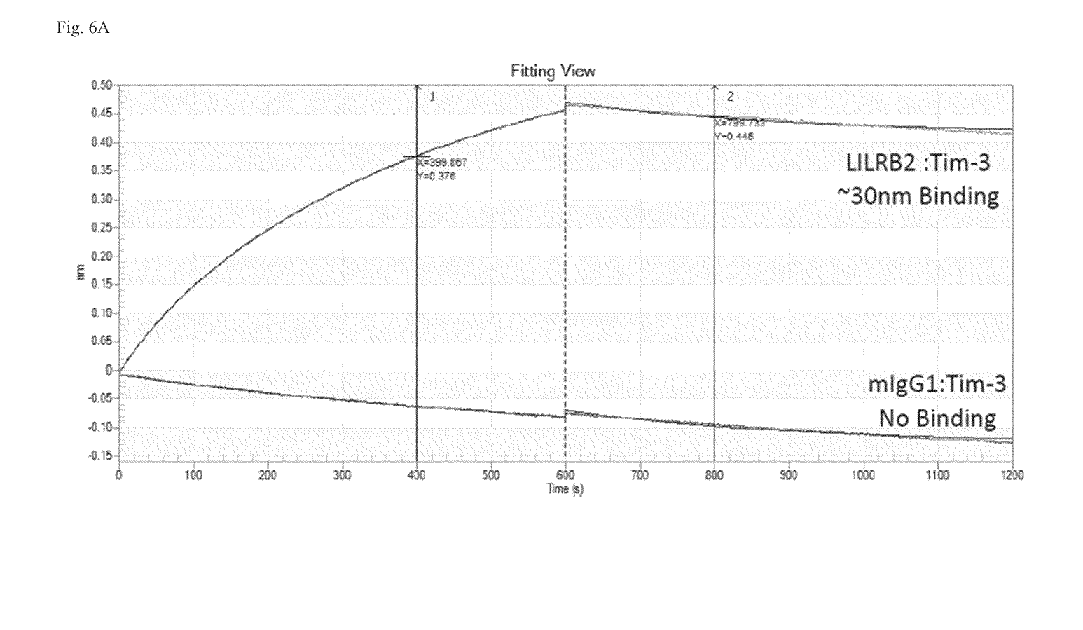

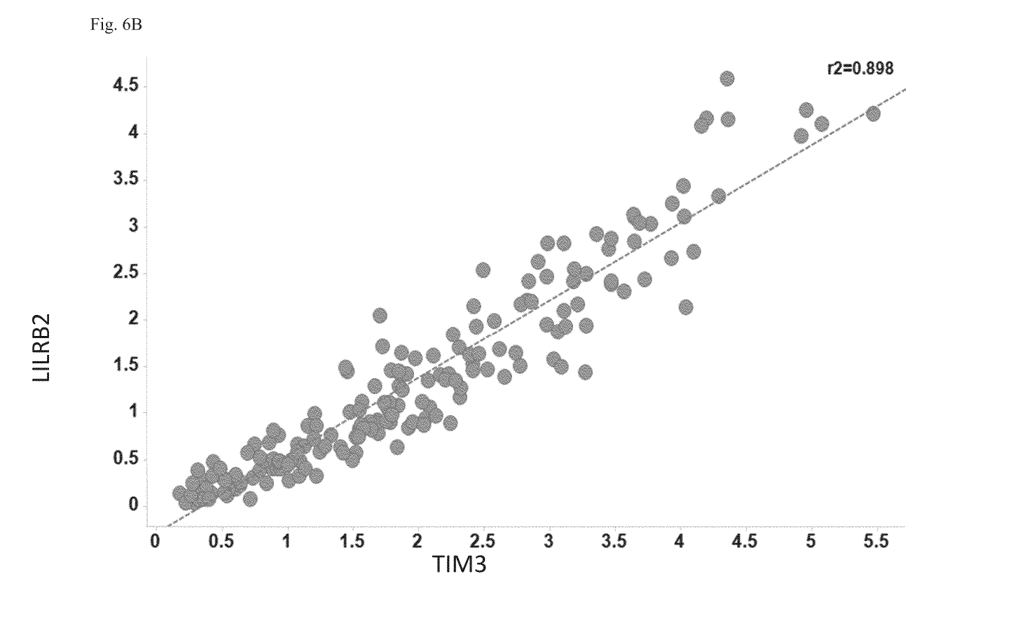

The macrophage and DC monoculture results with TIM-3 suggest that a relevant TIM-3 ligand is found on these cells as blockade of TIM-3 leads to functional consequences. Bioinformatic data were used to examine genes whose expression correlates with TIM-3 expression in human tumor samples. The list of expressed proteins was limited to surface receptors that could serve as a ligand for TIM-3. From this list, several candidate proteins were tested for binding to TIM-3. Of these candidates, LILRB2 bound to TIM-3 with an affinity of ˜30 nM (Table 2, FIG. 6A). LILRB2 was not previously reported as a counter receptor for TIM-3.

TABLE 2
Binding parameters

| Sample ID | Loaded | Response | KD2 | kon2 | kdis2 | Affinity (nM) |
|---|---|---|---|---|---|---|
| hTIM-3-HIS | mIgG1 | −0.0815 | | | | NB |
| hTIM-3-HIS | huILILRB2 | 0.4549 | 2.67E−08 | 1.24E+05 | 3.30E−03 | 26.7 |

### Methods

Binding affinity was determined by using the OctetRed 96 System with anti-Human IgG Fc capture biosensors (ForteBio, 18-5064, Menlo Park, Calif.) according to the manufacturer's instructions. Human LILRB2-Fc Chimera (R&D Systems, 2078-T4, Minneapolis, Minn.) was coated to anti-Human IgG Fc capture sensors at 10 μg/ml. Saturated sensors were then rinsed in Kinetics Buffer (PBS, 0.1% BSA, 0.02% Tween-20, 0.05% azide) and dipped in hTIM-3-HIS protein at 200 nM. Data were analyzed with Octet Data Analysis Software v. 8.0 (ForteBio). Association (kₒₙ) and Dissociation (k_{off}) rates were determined for each mAb with sensor background subtracted. Equilibrium dissociation constant (K_{D}) is the ratio k_{off}/kₒₙ, as determined by the Octet Analysis Software.

### High Correlation Between TIM-3 and LILRB2

RNA sequencing data from ˜7500 individual tumors was collected as part of The Cancer Genome Atlas project (National Cancer Institute at NIH, Bethesda, Md.). The sequencing data were normalized and processed for expression and mutational analysis by specialized software (OmicSoft, Cary, N.C.). TIM-3 transcript levels were correlated to LILRB2 transcript levels across all of the available tumor samples using MatLabR2013b software (Mathworks Inc., Natick, Mass.). A tight association between TIM-3 and LILRB2 levels were observed in multiple tumor types. A representative figure showing correlation (Corr(S)>0.8) between TIM-3 and LILRB2 mRNA levels in bladder cancer patients (n=412) is shown in FIG. 6B.

### Example 7

### mAb Blocking Data

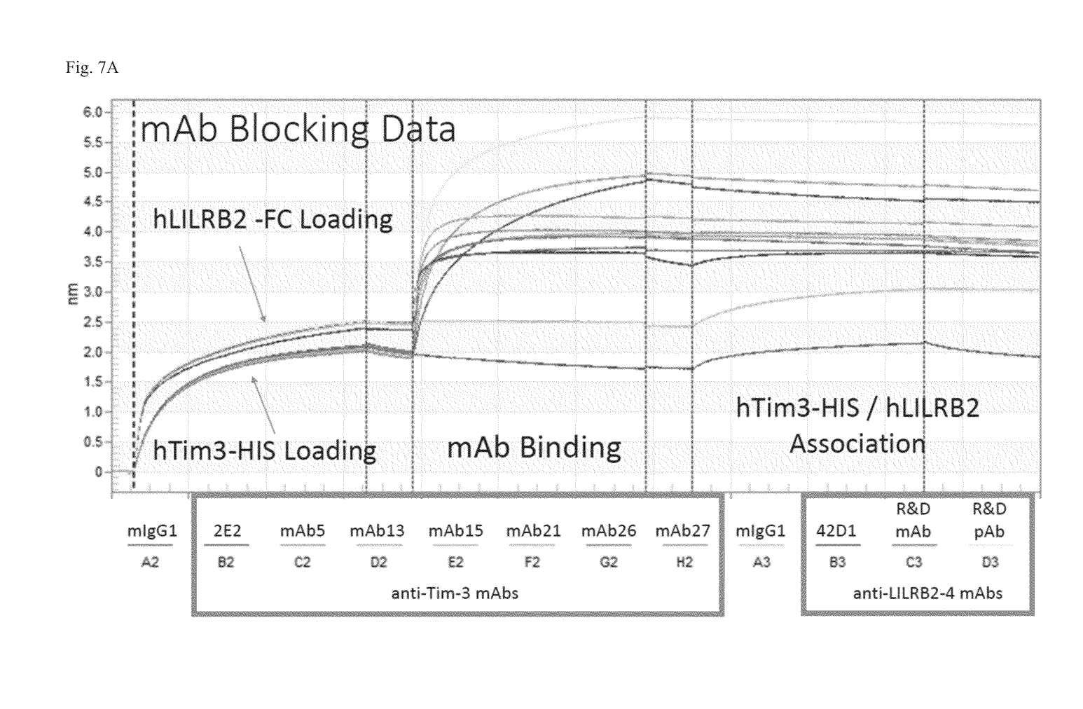

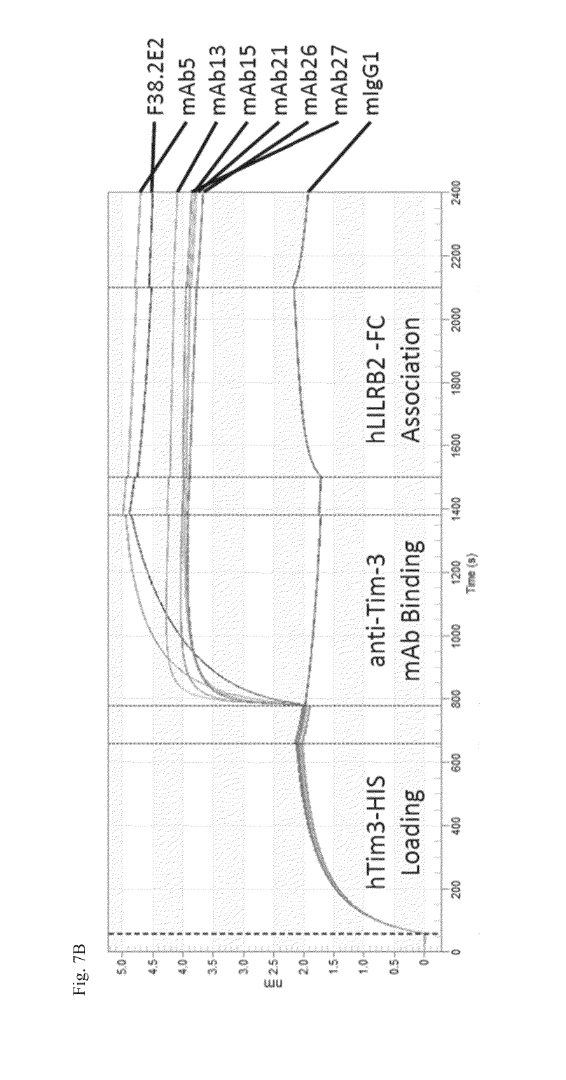

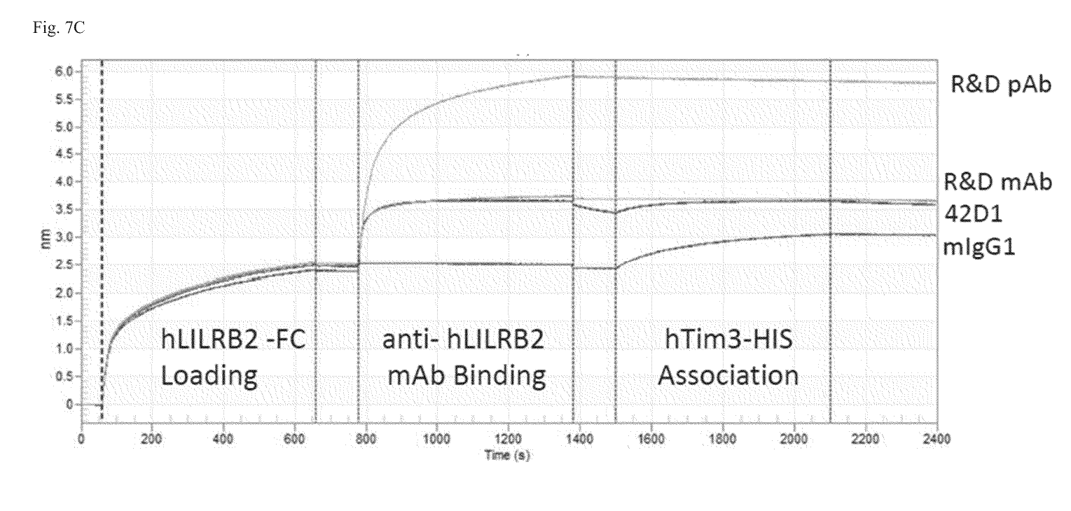

Several mAbs were tested in their ability to inhibit protein:protein binding of TIM-3 and LILRB2 (FIG. 7A). All TIM-3 specific mAbs tested blocked TIM-3 binding to LILRB2 regardless of their ability to block TIM-3 binding to the reported ligand Galectin-9 (FIG. 7B). Binding was only evaluated at a single high concentration with a select subset of TIM-3 antibodies. Conversely, the only anti-LILRB2 antibodies to block the TIM-3:LILRB2 interaction were the 287219 mAb (R&D Systems, MAB2078, Minneapolis, Minn.) and the polyclonal serum (R&D Systems, AF2078, Minneapolis, Minn.) (FIG. 7C). mAb42D1 clone (BioLegend, 338704, San Diego, Calif.), which was reported to bind human LILRB2, did not block the TIM-3:LILRB2 interaction. Therefore it appears that LILRB2:TIM-3 binding region is different but possibly overlapping with the Galectin-9 binding region.

### Methods

The ability of anti-TIM-3 and anti-LILRB2 Abs to block TIM-3:LILRB2 protein: protein binding was assessed by using the OctetRed 96 System. A) HIS-Tagged human TIM-3 ECD was coated to Nickel NTA sensors (ForteBio, 18-5102, Menlo Park, Calif.) at 20 μg/ml for 600 seconds. Saturated sensors were then rinsed in kinetics buffer and dipped in anti-TIM-3 mAbs (F38-2E2, JTx mAbs 5, 13, 15, 21, 26 and 27, or mouse IgG1 (MOPC-21, Biolegend, 400166, San Diego, Calif.)) at 150 nM for 600 seconds. Sensors were rinsed again in Kinetics buffer (PBS, 0.1% BSA, 0.02% Tween-20, 0.05% azide) and dipped into human LILRB2 ECD-Fc. B) human LILRB2 ECD-Fc was coated to anti-Human-Fc Capture (ForteBio) sensors at 30 μg/ml for 600 seconds. Saturated sensors were then rinsed in kinetics buffer and dipped in anti-LILRB2 mAbs (goat-anti-human Polyclonal Ab (R&D Systems), mouse anti-human mAb (clone 287219), anti-LILRB2 (clone 42D1) or mouse IgG1 (clone MOPC-21) at 150 nM for 600 seconds. Sensors were rinsed again in Kinetics buffer and dipped into human TIM-3-ECD-HIS. Data were analyzed with Octet Data Analysis Software v. 8.0. Blocking activity of each mAb was defined as a reduction in the calculated K_{D} compared to the ˜30 nM binding determined in Example 6).

### Example 8

### Differences Between Macrophages and DCs with TIM-3 Blockade

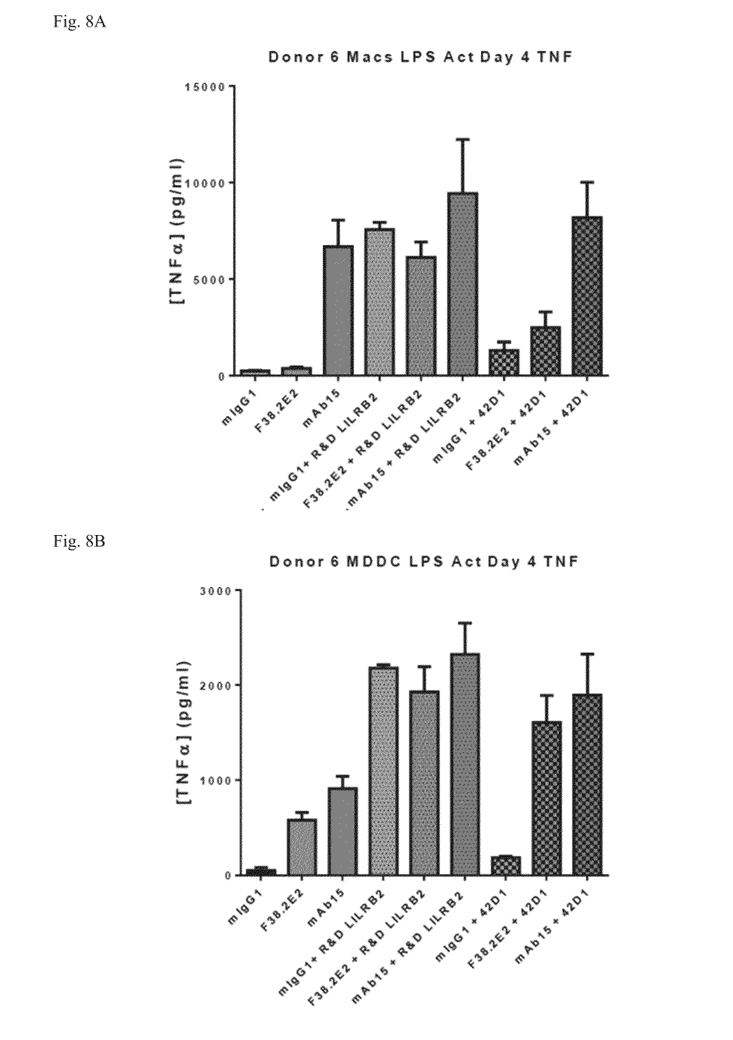

Blocking of TIM-3:LILRB2 interactions by antibodies was evaluated in activated-macrophage and activated-DC assays separately. In LPS-activated macrophages, both TIM-3 mAb15 and LILRB2 mAb 287219 were able to initiate TNFα secretion to the same magnitude (FIG. 8A). In contrast, in LPS-activated DC cultures, anti-LILRB2 mAb 287219 showed greater impact on TNFα secretion in comparison to the anti-TIM-3 activity of mAb15 (FIG. 8B). In DC cultures the more pronounced activity of anti-LILRB2 in comparison to anti-TIM-3 suggests that its activity may be attributed to inhibiting LILRB2:TIM-3 activity as well as LILRB2 with HLA-G or other MHC Class I-like molecules. Cumulatively these data suggest that the change in macrophage activity affected by TIM-3 blocking antibodies is attributed to blocking TIM-3:LILRB2 interactions and not TIM-3 interactions with other reported ligands such as Gal-9.

### Methods

PBMCs were isolated by Ficoll separation from 100 ml of fresh whole blood from two donors. CD14 Negative selection was carried out on all cells from each donor according to manufacturer's protocol. 1 million cells per well added to a 6-well plate in MDDC media (RPMI with 10% FBS, 20 ng/ml rhIL-4, 20 ng/ml rhGM-CSF) or Macrophage media (RPMI with 10% FBS, 50 ng/ml rhM-CSF). Media was changed at culture days 2, 4 and 6. On culture day 8, DCs were harvested, pooled, counted and were assessed by flow cytometry for expression of CD14, MHC-II, CD86 and CD209. At least 80% of MDDCs were positive for CD86, CD209 HLA-DR and TIM-3. MDDCs did not express LILRB2 prior to activation. At least 90% of macrophages were positive for CD14, CD86, HLA-DR, TIM-3 and did express LILRB2. DCs or macrophages were arrayed in fresh RPMI 10% FBS at 10-20,000 per well in 100 μl in a 96 well round bottom plates as outlined above. 100 μl of RPMI containing 200 ng/ml LPS was added to each well, as indicated. TIM-3 specific mAbs F38-2E2, JTx mAbs or mouse IgG1 Isotype was added at 50 μg/ml. Anti-LILRB2 mAbs (R&D Systems clone 287219 or clone 42D1) were added at 50 μg/ml and 10 μg/ml respectively. After 4 days of LPS activation, supernatant was collected and frozen at −20° C., DCs were dissociated from the plate, washed once in PBS with 2% FBS, and stained for surface expression of CD209, MHC-II, CD80, CD86 and CD11c. Supernatant cytokine concentration was measured using multi-parameter cytokine bead array. Data are representative of DCs from 6 healthy donors in 3 experiments.

### Example 9

### Response of HMGB1- and CD40L-Activated Peripheral Blood Mononuclear Cells Respond to Anti-TIM-3 Blockade

The examples described above showed modulation of macrophage and DC biology using cells activated with LPS. In the present example, macrophages activated by a tumor-derived activator, HMGB1, and an adaptive immune system ligand, CD40L, were evaluated.

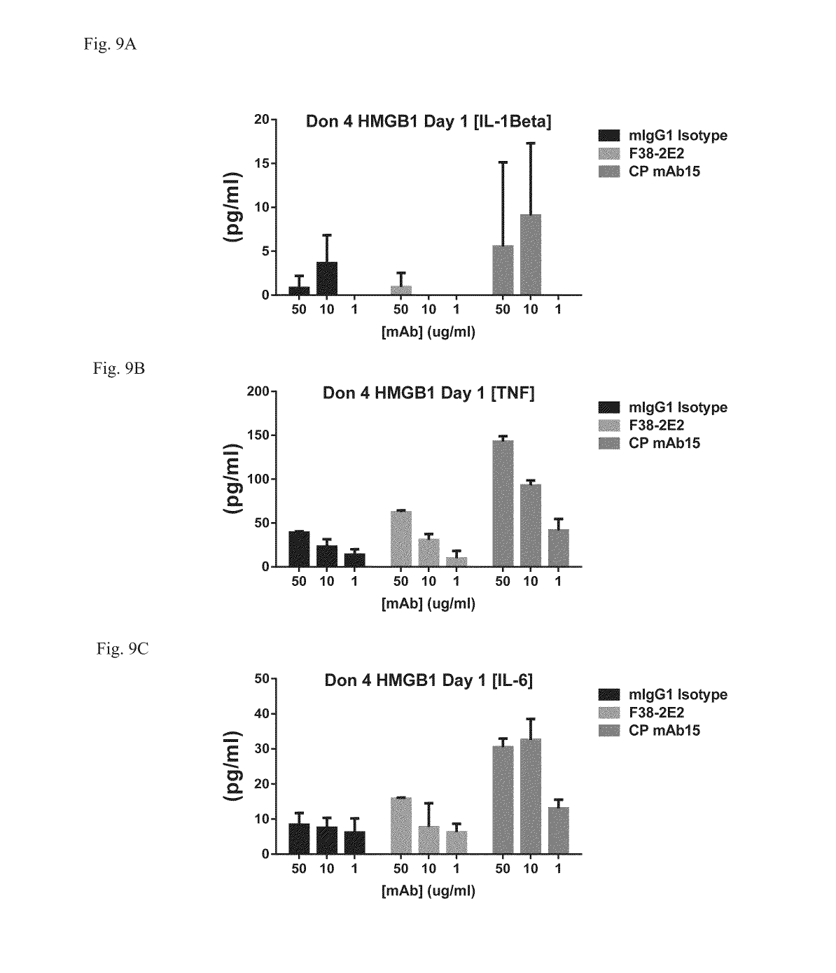

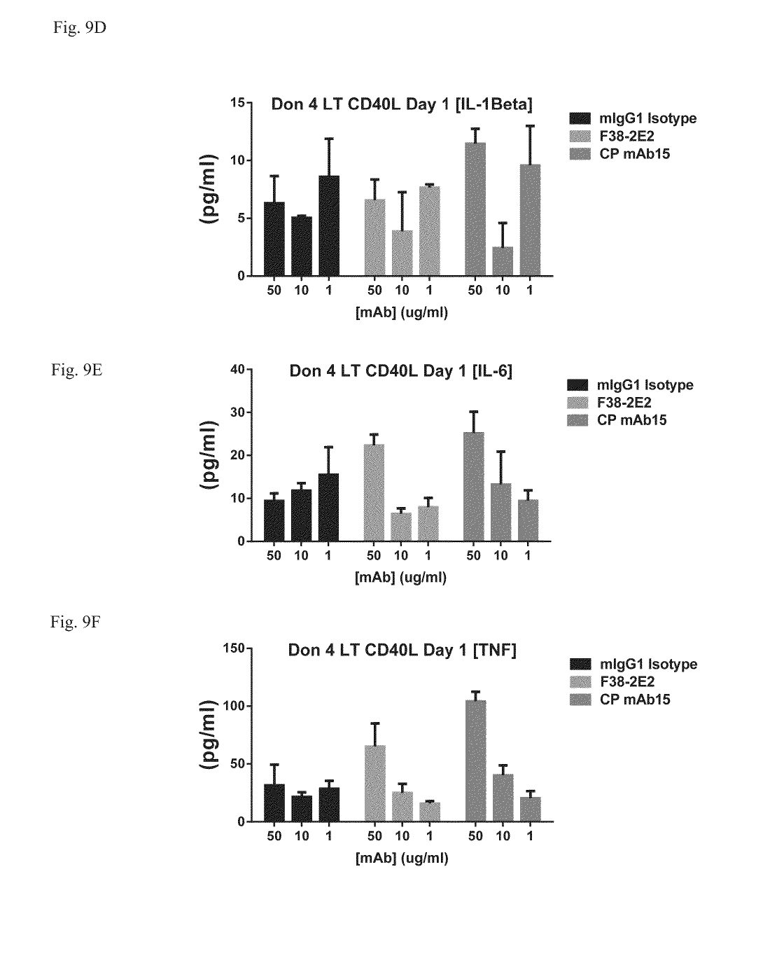

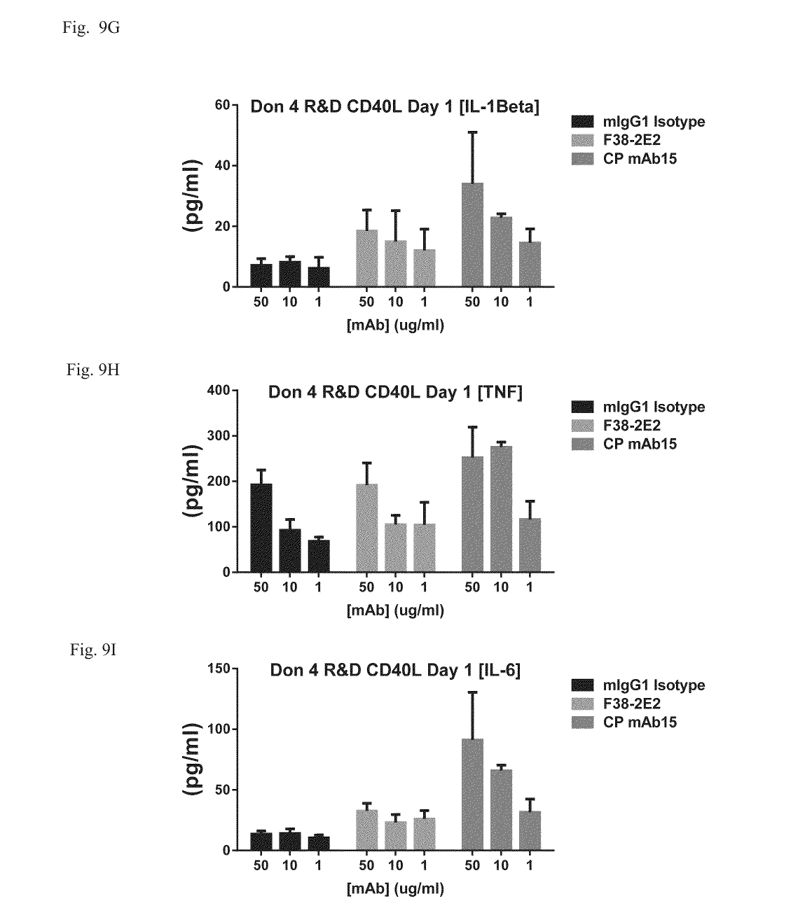

CD14+ Monocytes from fresh blood were cultured for 7 days for using M-CSF (50 ng/ml) in RPMI with 10% FBS. Macrophages were stimulated with 1 μg/ml of recombinant human HMGB1 (R&D Systems, 1690-HMB-050, Minneapolis, Minn.), or 500 ng/ml recombinant human CD40-Ligand (R&D Systems, 6420-CL-025/CF, Minneapolis, Minn. or ThermoFisher, PHP0024, Grand Island, N.Y.) on Day 6. Anti-TIM-3 mAbs were added at 50, 10 or 1 μg/ml. The anti-TIM-3 antibodies were antibody F38-2E2 and mAb15, described above. The negative control was mIgG1 isotype control. Supernatants were collected after 24 h and cytokines were measured using Cytometric Bead Arrays. Data presented in FIGS. 9A-9I are representative of 1 healthy donor.

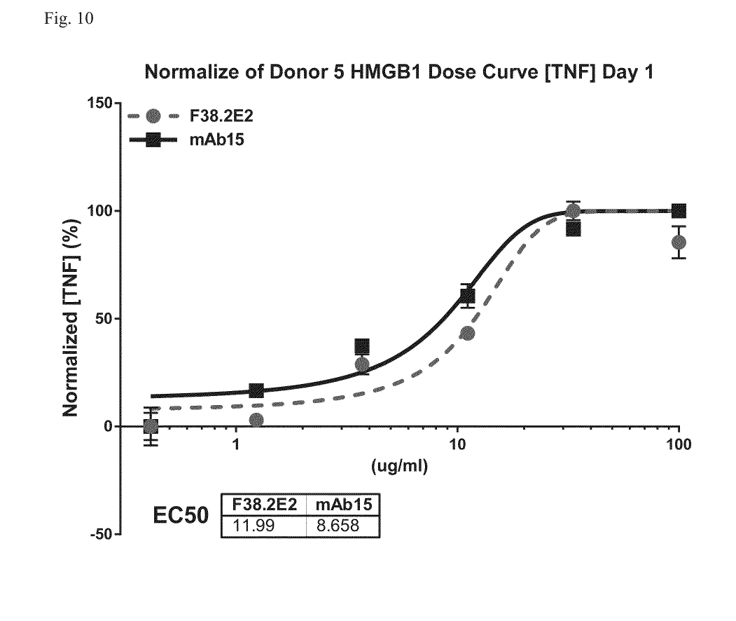

Dose curves using HMGB1 activated macrophages and anti-TIM-3 antibodies were evaluated (FIG. 10). CD14+ Monocytes from fresh blood were cultured for 7 days for using M-CSF (50 ng/ml) in RPMI with 10% FBS. Macrophages were stimulated with 1 μg/ml of recombinant human HMGB-1 on day 7, anti-TIM-3 mAbs or isotype were added at the indicated concentrations. Supernatants were collected after 24 h and TNFα levels were measured using Cytometric Bead Arrays. The results show mAb15 blocking of TIM-3 was more effective at stimulating the expression of TNFα compared to antibody F38-2E2. Data are representative of 1 healthy donor.

### Example 10

### Dose Curves for Anti-TIM-3 or Anti-LILRB2

PBMCs were isolated by Ficoll separation from 100 ml of fresh whole blood from two donors. CD14 Negative selection was carried out on all cells from each donor according to manufacturer's protocol. 1 million cells per well added to a 6-well plate in Macrophage media (RPMI with 10% FBS, 50 ng/ml rhM-CSF). Media was changed at culture days 2, 4 and 6. At least 90% of macrophages were positive for CD14, CD86, TIM-3 and LILRB2. Macrophages were arrayed in fresh RPMI 10% FBS at 10-20,000 per well in 100 μl in 96-well round bottom plates as outlined above. 100 μl of RPMI containing 200 ng/ml LPS was added to each well, as indicated. TIM-3 specific mAbs F38-2E2, mAb15, anti-LILRB2 mAb or mouse IgG1 isotype was added at the indicated concentrations. Supernatant was collected and frozen at −20° C. on day 1, day 2 and day 3. Supernatant cytokine concentration was measured using multi-parameter cytokine bead array. Data are representative of 3 healthy donors.

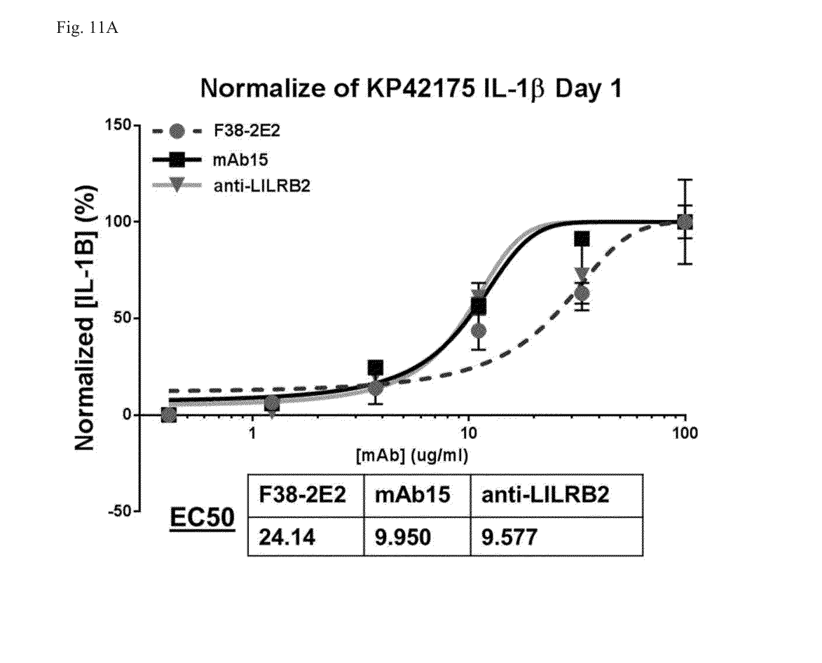

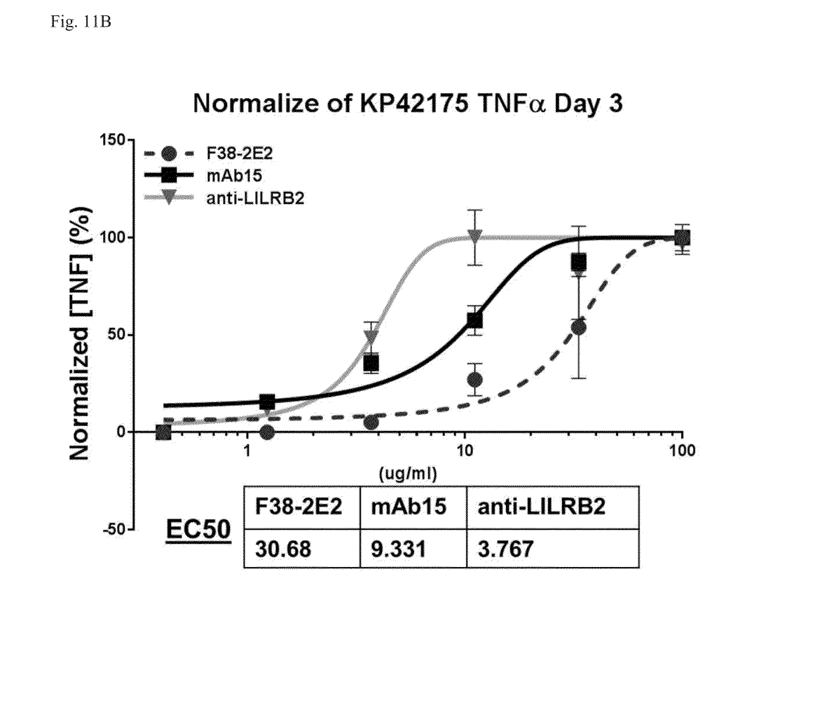

IL-1β results for day 1 are shown in FIG. 11A and TNFα results for day 3 are shown in FIG. 11B. Results show that blockage of TIM-3:LILRB2 interactions by either anti-TIM-3 antibodies of anti-LILRB2 antibodies resulted in the expression of IL-1β or TNFα.

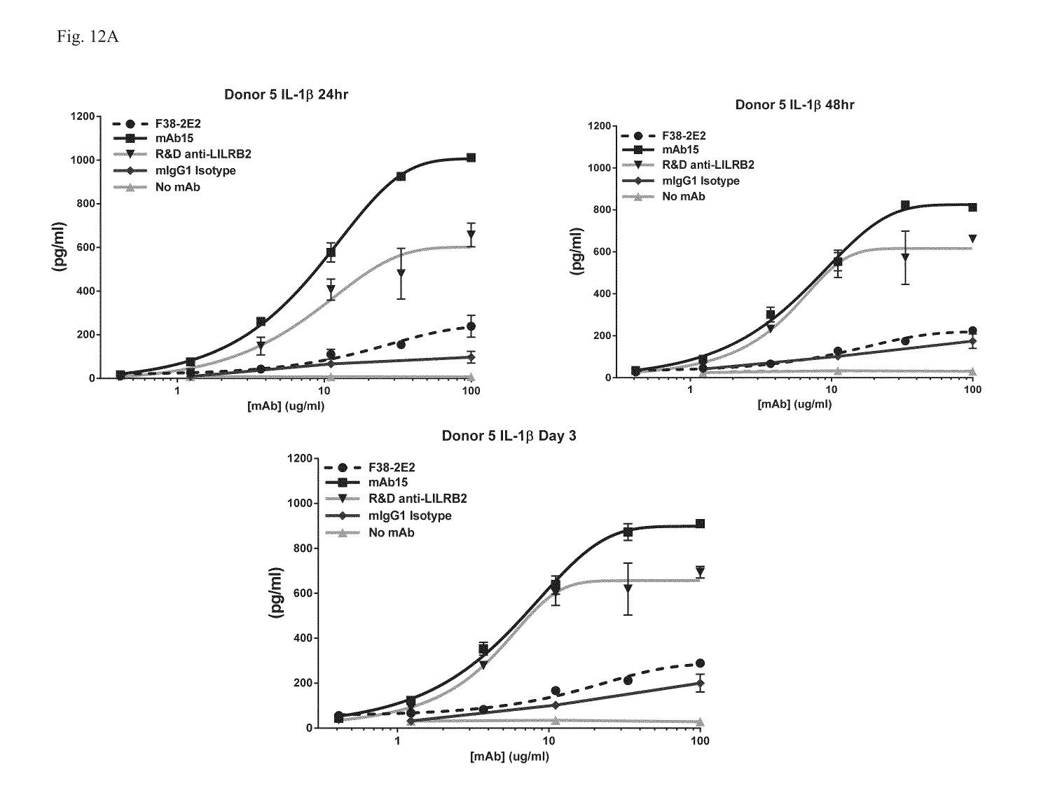

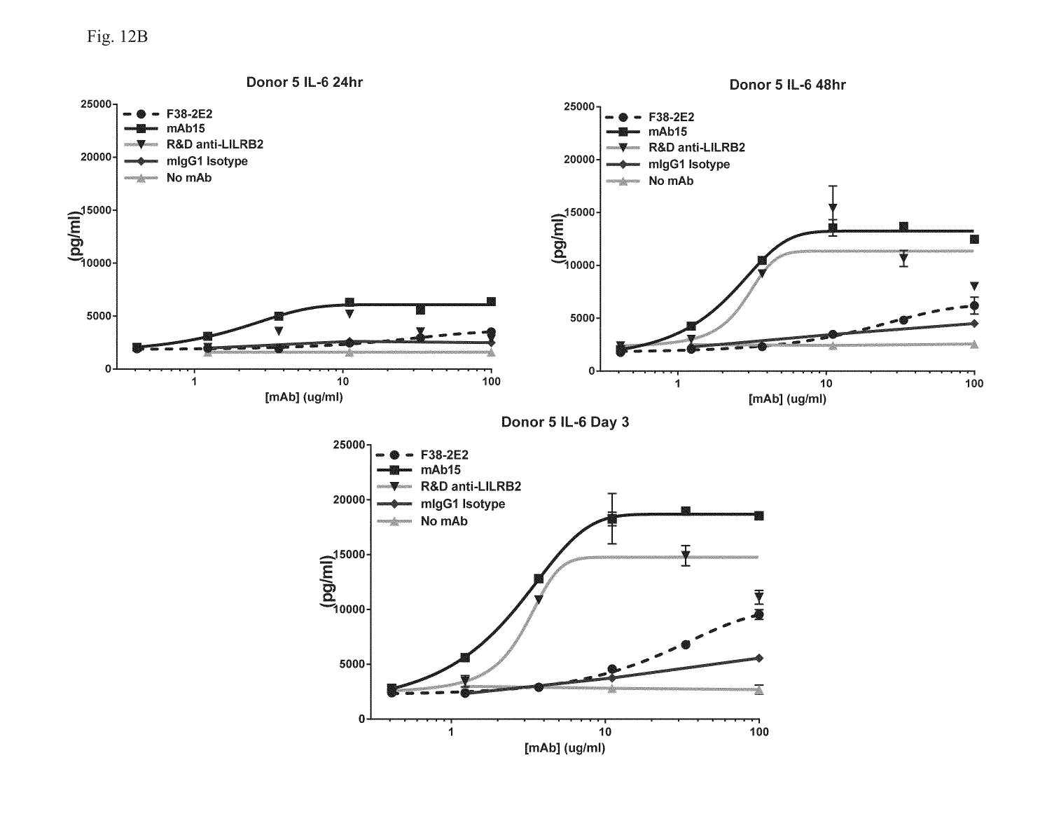

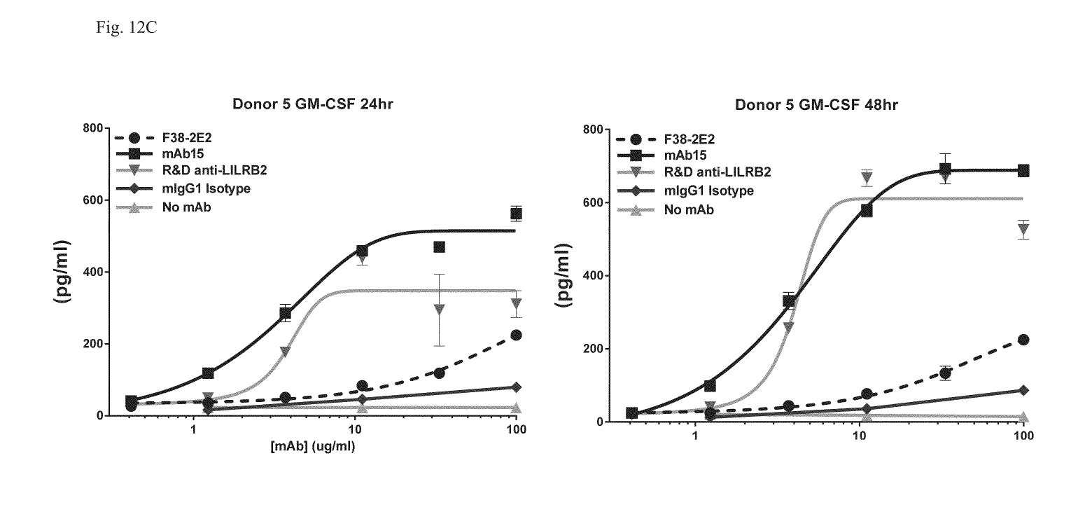

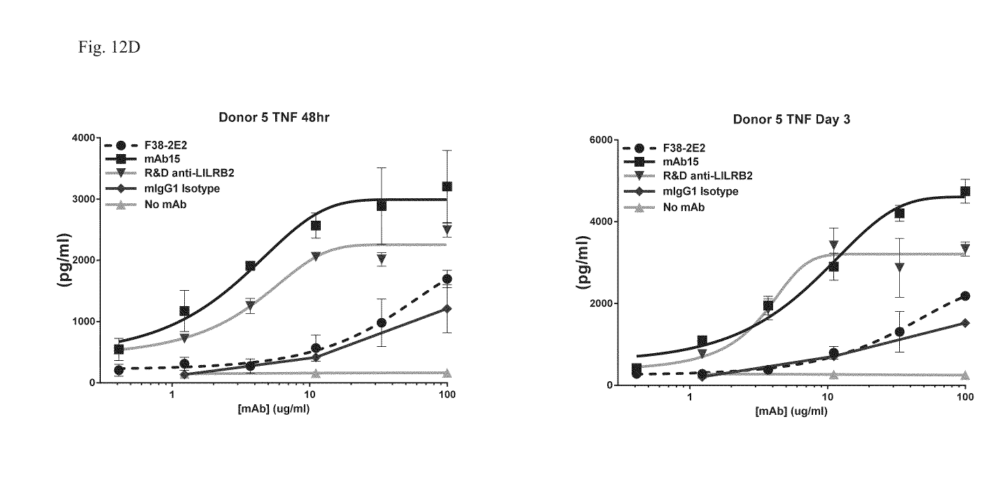

FIG. 12 shows a time course of expression of IL-1β(FIG. 12A), IL-6 (FIG. 12B), GM-CSF (FIG. 12C) and TNFα (FIG. 12D). Results show early expression of cytokines following block of TIM-3:LILRB2 interactions (e.g., by day 3).

### Example 11

### Cytokine Expression from Macrophages from Donor with Low LILRB2 Expression

PBMCs were isolated by Ficoll separation from 100 ml of fresh whole blood from two donors. CD14 Negative selection was carried out on all cells from each donor according to manufacturer's protocol. 1 million cells per well added to a 6-well plate in Macrophage media (RPMI with 10% FBS, 50 ng/ml rhM-CSF). Media was changed at culture days 2, 4 and 6. At least 90% of macrophages from Donor KP42331 were positive for CD14, CD86, TIM-3 and LILRB2, while Macrophages from Donor KP42334 were positive for CD14, CD86 and TIM-3, but expressed low levels of LILRB2. A sample of macrophages from both of these donors was assessed for surface expression of other inhibitory members of the LILRB family. Macrophages were incubated in TruStain FcX (Biolegend, 422302, San Diego, Calif.) then labeled with mAbs specific for LILRB1 (R&D Systems MAB20171, Minneapolis, Minn.), LILRB2 (clone 287219), LILRB3 (R&D Systems, MAB1806-100, Minneapolis, Minn.), LILRB4 (R&D Systems, MAB24251, Minneapolis, Minn.) and LILRB5 (R&D Systems, MAB3065, Minneapolis, Minn.) in PBS with 2% FBS for 20 minutes on ice, washed once and fixed in PBS with 2% PFA for analysis by flow cytometry. Macrophages were arrayed in fresh RPMI 10% FBS at 10-20,000 per well in 100 μl in 96-well round bottom plates as outlined above. 100 μl of RPMI containing 200 ng/ml LPS was added to each well, as indicated. TIM-3 specific mAbs F38-2E2, mAb15 or mouse IgG1 Isotype was added at 10 μg/ml. Supernatant was collected and frozen at −20° C. on day 1 and day 2. Supernatant cytokine concentration was measured using multi-parameter cytokine bead array.

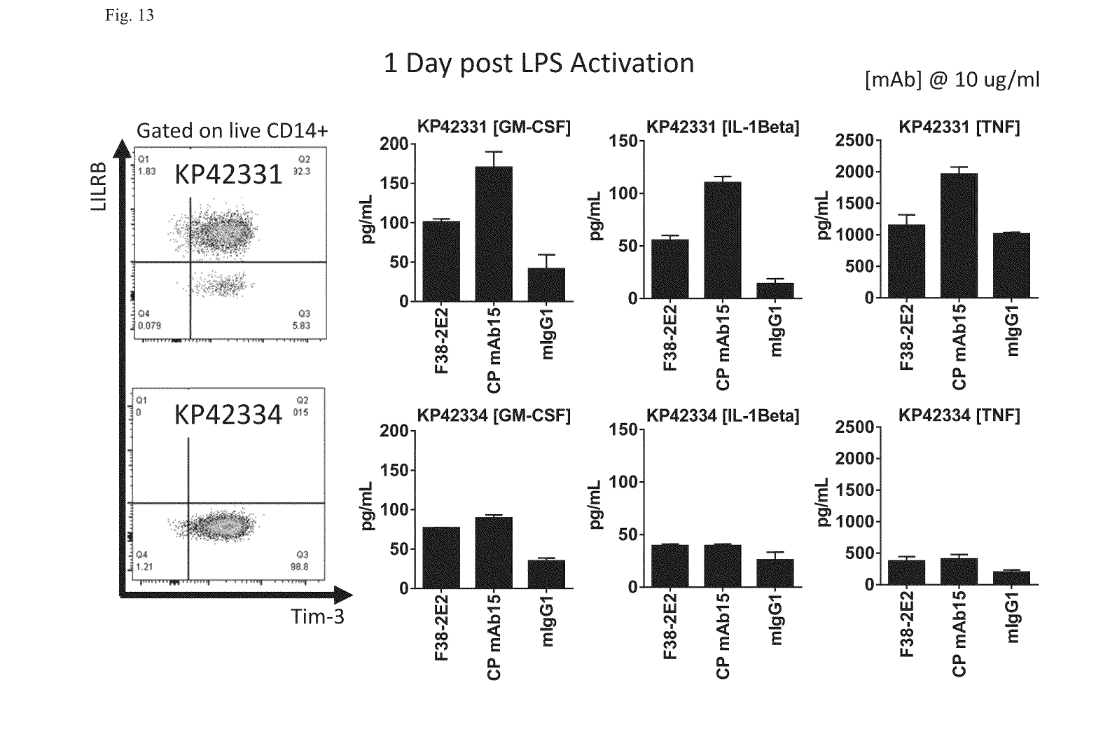

Macrophages from a donor with low LILRB2 showed diminished modulation of GM-CSF, IL-1β, and TNFα expression with mAb15 compared to F38-2E2 (FIG. 13).

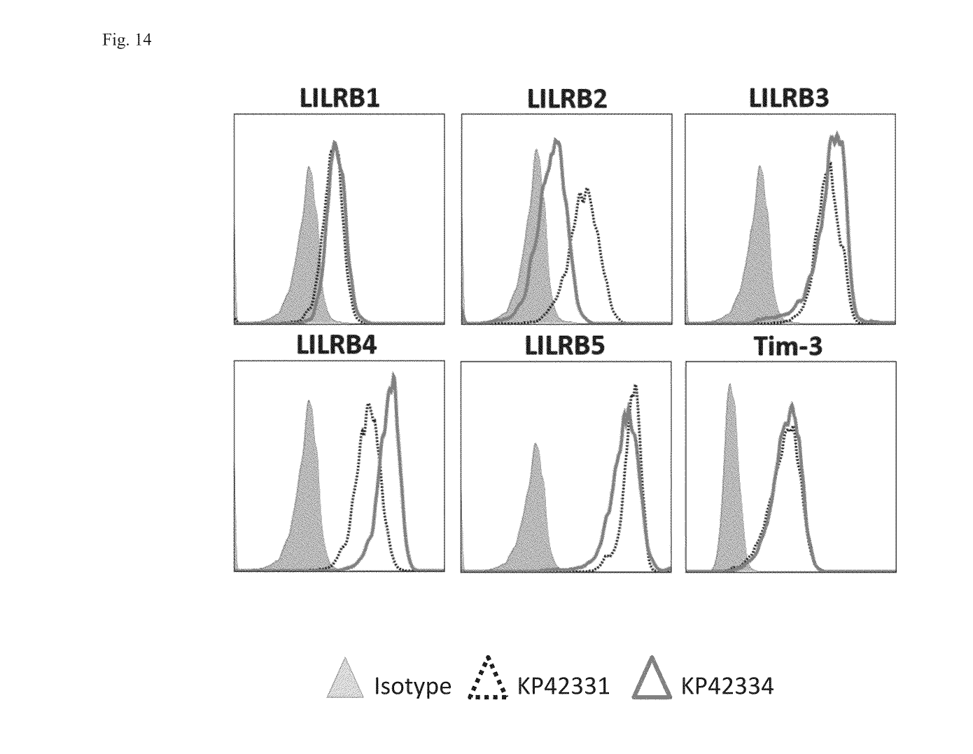

To demonstrate that the donor specifically had diminished expression of LILRB2, macrophages from this donor, as well as from a donor that expressed LILRB2 were assayed for expression of a number of LILRB family proteins as well as TIM-3. As shown in FIG. 14, this donor specifically expressed LILRB2 at low levels.

Modulation of various cytokines following treatment of activated macrophages from donors expressing LILRB2 at normal levels or at low levels is shown in FIGS. 15A and 15B. mAb15 stimulation of pro-inflammatory cytokines GM-CSF, IL-1α, IL-1β, IL-6 and TNFα was greater for macrophages from the LILRB2+ donor compared to the low LILRB2 donor (FIG. 15B, compare top panels to bottom panels). Expression of other cytokines showed little difference in cytokine expression upon mAb15 treatment of activated macrophages from both the LILRB2+ donor and low LILRB2 donor. Secretion of cytokines IL-10, CCL2, CCL3, and CCL5 decreased upon treatment with mAb15 (FIG. 15B). Once again, the response to treatment with mAb15 was greater for macrophages the LILRB2+ donor compared to macrophages from the low LILRB2 donor.

### Example 12

### Domain Swapping of TIM-3

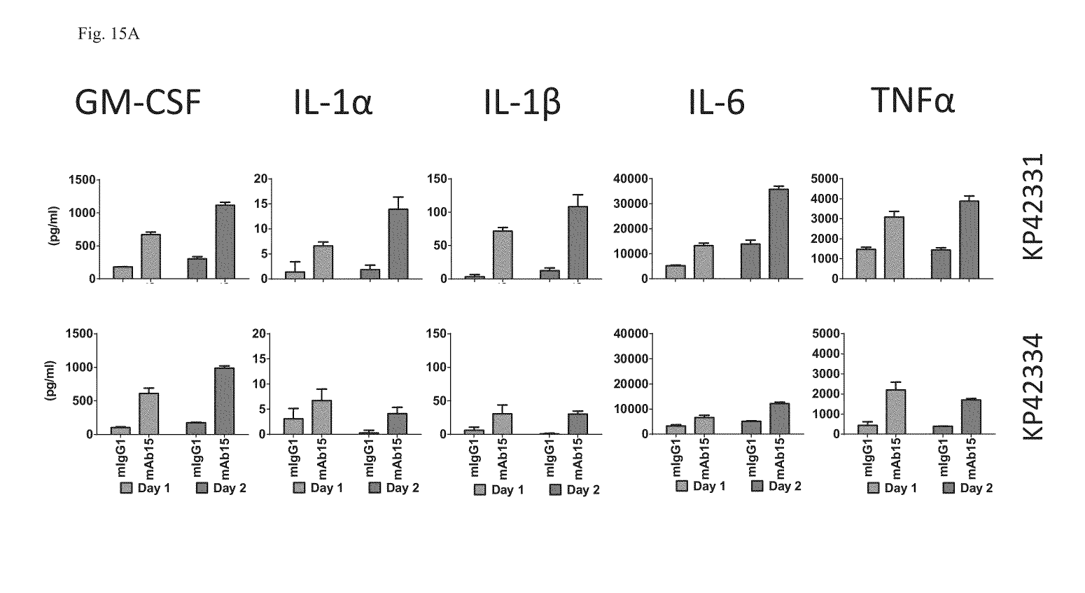

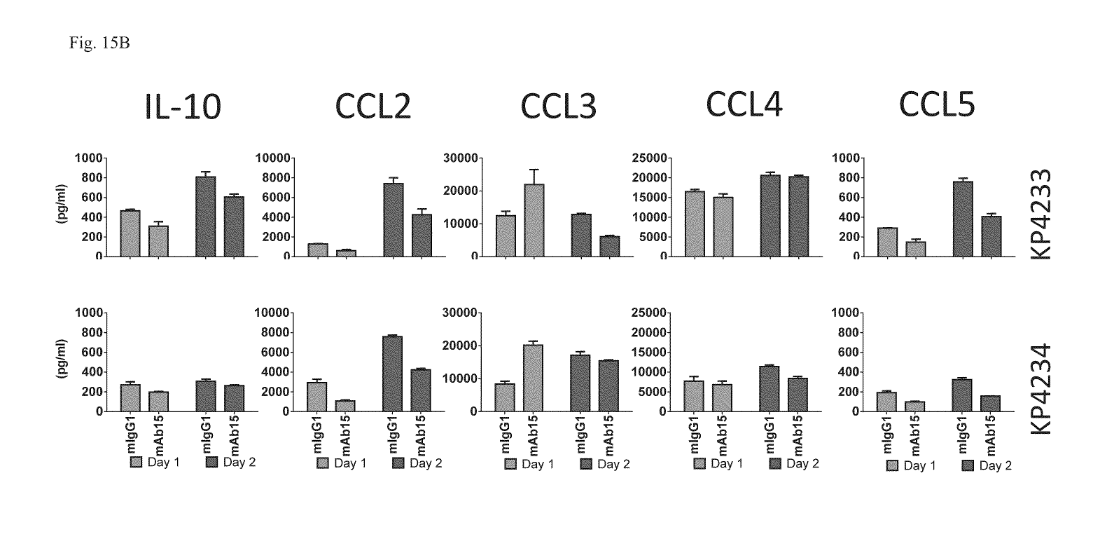

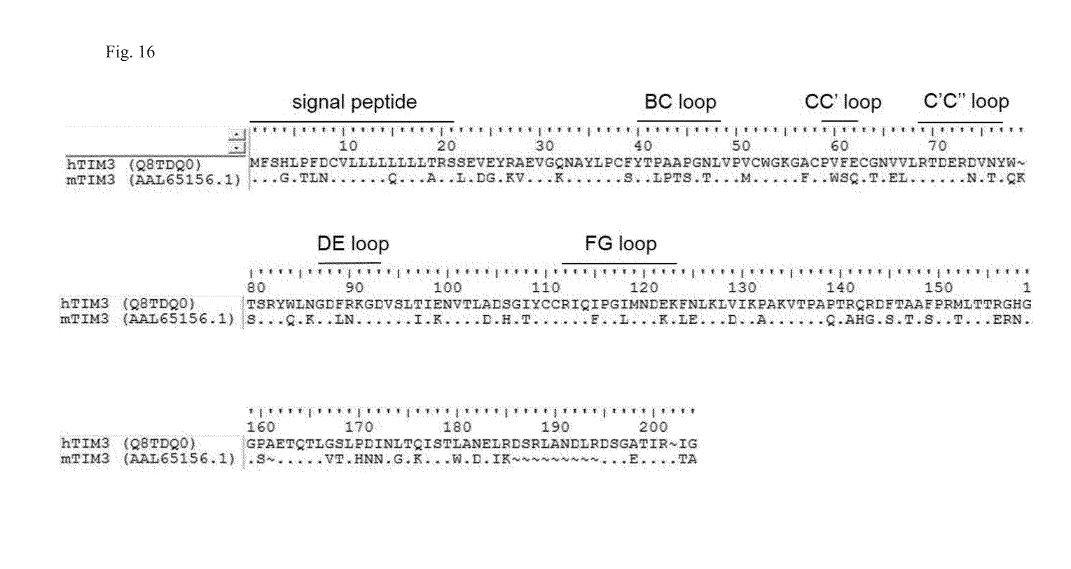

In an effort to determine the binding domains of anti-TIM-3 antibodies, various domains of human TIM-3 were replaced by the corresponding mouse TIM-3 domain. A sequence alignment of human TIM-3 and mouse TIM-3 is shown in FIG. 16. The human TIM-3 domains that were replaced by mouse TIM-3 include the BC loop, the CC′ loop, the C′C″ loop, the DE loop, and the FG loop. Expression constructs were made for each of the domain swaps. Variant proteins were expressed and purified on a small scale and assayed for mAb binding by ELISA. Potential epitopes are identified by a decrease in binding for a particular swap.

The expression cassette was the human TIM-3 ECD (Ser22-Arg200, Accession #:Q8TDQ0) with substitutions from the mouse TIM-3 ECD (Accession #:Q8TDQ0) to generate chimeric proteins. All wildtype and chimeric ECD versions (SEQ ID Nos: 63-69) of TIM-3 were fused to the human IgG1 Fc. HEK 293F cells were transiently transfected in shake flasks. Supernatants were harvested and fusion proteins were purified using MabSelect resin (GE Healthcare Life Sciences, 17-5199-01, Pittsburgh, Pa.).

The vectors used were TBH003.pCP-VKL-hTIM-3 ECD linker-Fc and TBH004.pCP-VKL-mTIM-3 ECD linker-Fc. The ORF contains: human Ig kappa signal peptide; human TIM-3 ECD (22-202, Accession #:Q8TDQ0) or mouse TIM-3 ECD Accession #:Q8TDQ0; and human IgG1 Fc. Expression vector features include a pEF1a promoter, SV40 polyA signal, a gene for ampicillin resistance, a pUC origin of replication and a viral origin of replication.

The ELISA protocol was as follows. Nunc Maxisorp plates were coated with 50 μl of capture (hTIM-3-hFc) at 4 μg/ml in D-PBS, and incubated overnight at 4° C. Plates were washed three times with PBS-0.05% Tween-20 (PBS-T). Plates were blocked for 1 hr at room temperature with 200 μl of PBS-T+1% BSA. Plates were washed three times with PBS-T. Fifty μl of mAb diluted in TBS-T was added per well, and incubated 1 hr at room temperature. Plates were washed with PBS-T. One hundred μl of secondary HRP-conjugate in PBS-T (streptavidin-HRP, 1:10,000) were added and plates were incubated (covered) for 1 hr at room temperature. Plates were washed with PBS-T. One hundred μl TMB substrate (Pierce) was added per well and plates were incubated at room temperature until color developed. Reactions were stopped with 100 μl of 2M sulfuric acid. Absorbance at 450 nm was measured. Results are presented in Table 3.

TABLE 3
Binding of anti-TIM-3 antibodies to domains of TIM-3

| | | F38- | | | | | | | |
|---|---|---|---|---|---|---|---|---|---|
| Construct | mAb5 | 2E2 | mAb15 | mAb13 | mAb17 | mAb22 | mAb48 | mAb58 | mAb91 |
| hTIM3-Fc | 1.51 | 1.337 | 1.781 | 2.189 | 2.076 | 2.129 | 1.388 | 1.39 | 1.568 |
| WT | | | | | | | | | |
| hTIM3-Fc | 1.379 | 1.229 | 1.683 | 2.171 | 2.073 | 2.057 | 1.3 | 1.835 | 1.442 |
| murine BC | | | | | | | | | |
| loop | | | | | | | | | |
| hTIM3-Fc | 1.421 | 0.094 | 0.501 | 1.043 | 0.926 | 0.91 | 0.247 | 1.392 | 0.502 |
| murine CC′ | | | | | | | | | |
| loop | | | | | | | | | |
| hTIM3-Fc | 1.577 | 1.484 | 1.758 | 0.53 | 0.19 | 0.141 | 0.045 | 0.338 | 0.049 |
| murine C′C″ | | | | | | | | | |
| loop | | | | | | | | | |
| hTIM3-Fc | 1.511 | 0.978 | 1.295 | 0.069 | 0.076 | 0.066 | 0.047 | 0.855 | 1.015 |
| murine DE | | | | | | | | | |
| loop | | | | | | | | | |
| hTIM3-Fc | 1.66 | 1.48 | 1.983 | 2.374 | 2.318 | 2.221 | 1.533 | 1.897 | 1.737 |
| murine FG | | | | | | | | | |
| loop | | | | | | | | | |

mAb F38-2E2 and mAb15 bind strongly to the CC′ loop and mildly to the DE loop. These mAbs do not bind consecutive loops. However, mAb13, mAb17, mAb22, mAb48, mAb58 and mAb91 bind strongly to the consecutive C′C″ and DE loops and mildly to the CC′ loop.

### Example 13

### Functional Activity of Anti-TIM-3 Antibodies

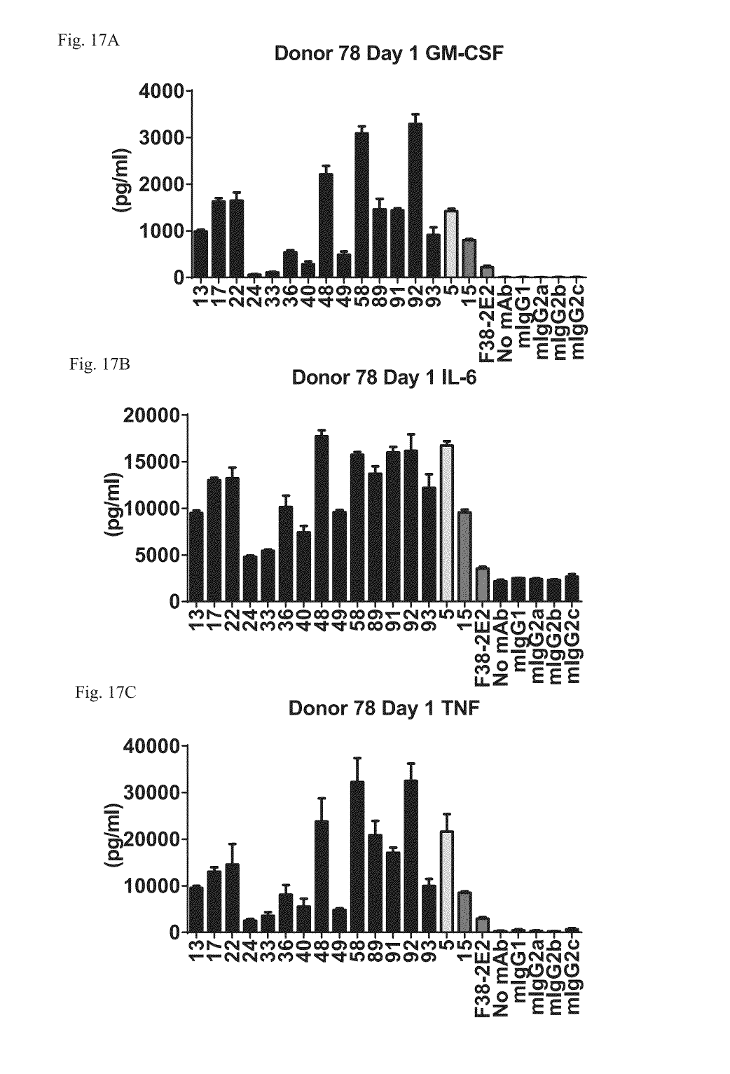

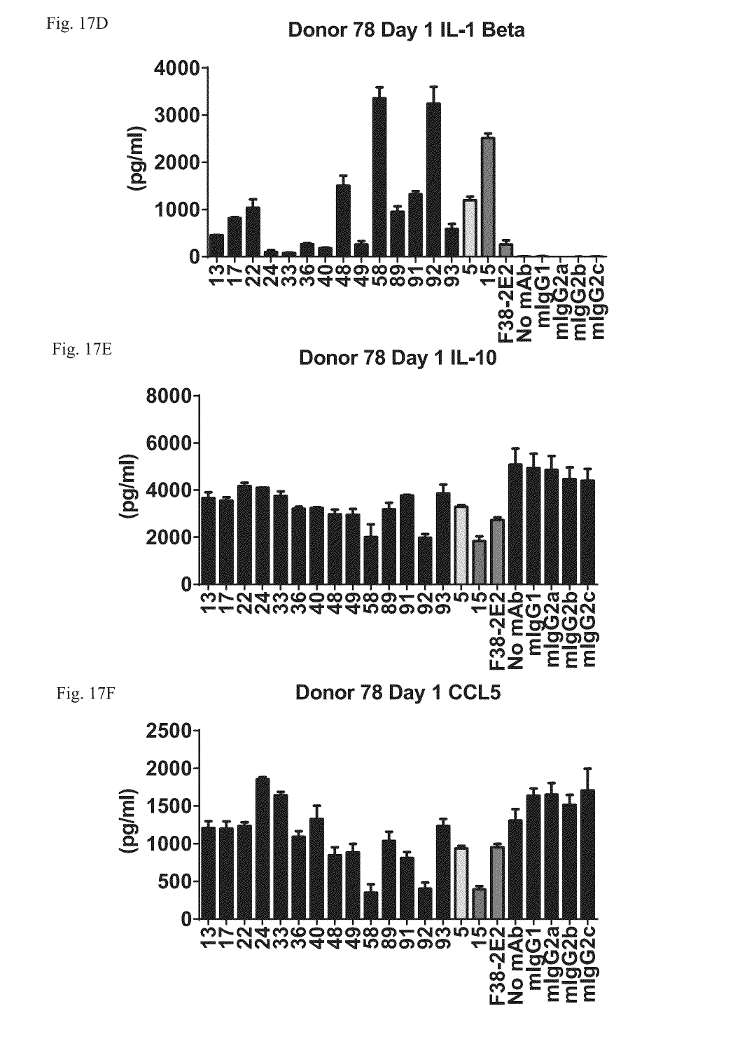

A sample of mAbs generated in the initial mouse immunization screen were tested in the macrophage activation assay. In this assay, 50,000 macrophages per well were arrayed into a 96-well round bottom plate, activated with 100 ng/ml LPS in the presence of mouse anti-human TIM-3 antibodies at 25 μg/ml as described above. Macrophages were obtained from two different donors. Cytokine concentrations were measured 24 hours post-activation as described above. Results are presented in FIGS. 17A-17F.

About half of the mouse hybridoma antibodies in the mAb13 bin (FIG. 1B), including mAb13, mAb17, mAb22, mAb58, mAb48, and mAb91, showed functional activity as measured by expression of GM-CSF, IL-6, TNFα and IL-1β; for example, compared to mAb15 and/or F38-2E2. Most mAbs that showed functional activity related to pro-inflammatory cytokines showed decreased expression of T cell suppressor functions as measured by IL-10 and CCL5 (FIGS. 17E and 17F).

In a second assay 100,000 M-CSF differentiated macrophages per well were arrayed into a 96-well round bottom plate, activated with 100 ng/ml LPS in the presence of mouse anti-human TIM-3 antibodies at 25 μg/ml. Macrophages were obtained from two different donors. Cytokine concentrations were measured at 24 hours post-activation to validate activity. Triplicate wells were pooled to obtain sufficient RNA at concentration for the assay. Cells were pelleted and lysed in RLT buffer (Qiagen) and RNA was collected using the RNEasy Miniprep kit (Qiagen, #74106, Hilden, Germany) RNA was quantified on a NanoDrop and a maximum of 100 ng was used for analysis. A custom panel capable of interrogating ˜600 genes was assembled by Nanostring Technologies (Seattle, Wash.) and analyzed on the nCounter system at the Dana-Farber Cancer Institute's Molecular Biology Core Facility. The data were normalized using standard methods and genes that were upregulated or downregulated >1.5 fold (2 standard deviations) in the mAb15 versus isotype control groups were highlighted. Anti-TIM-3 blockade induced a pro-inflammatory state as evidenced by the upregulation of genes like TNF-α, IL-6, GM-CSF, CXCL2 and the downregulation of genes like TGFB1, CD163 (FIG. 17G).

### Example 14

### Mixed Lymphocyte Reaction

Stimulation of expression of cytokines by macrophages by treatment with antibodies F38-2E2 and mAb15 was evaluated in a mixed lymphocyte reaction (MLR) assay. PBMCs were isolated by Ficoll separation from 100 ml of fresh whole blood from two donors. CD14 Negative selection was carried out on all cells from each donor according to manufacturer's protocol. 1 million cells per well added to a 6-well plate in Macrophage media (RPMI with 10% FBS, 50 ng/ml rhM-CSF). Media was changed at culture day 2, 4 and 6. At least 90% of macrophages were positive for CD14, CD86, TIM-3 and LILRB2.

Macrophages were arrayed in fresh RPMI 10% FBS at 10,000 or 100,000 per well in 100 μl containing 100 ng/ml LPS in 96-well round bottom plates as outlined above. TIM-3 specific mAbs F38-2E2, mAb15 or mouse IgG1 Isotype was added at indicated concentrations.

Allogeneic T Cells were purified from a frozen bank of human PBMCs by Negative selection, labeled with CFSE, and 100,000 cells were added in 100 μl to macrophages for MLR.

Supernatant was collected and frozen at −20° C. on day 1, day 4 and day 7. Supernatant cytokine concentration was measured using multi-parameter cytokine bead array. Data are representative of 3 healthy donors.

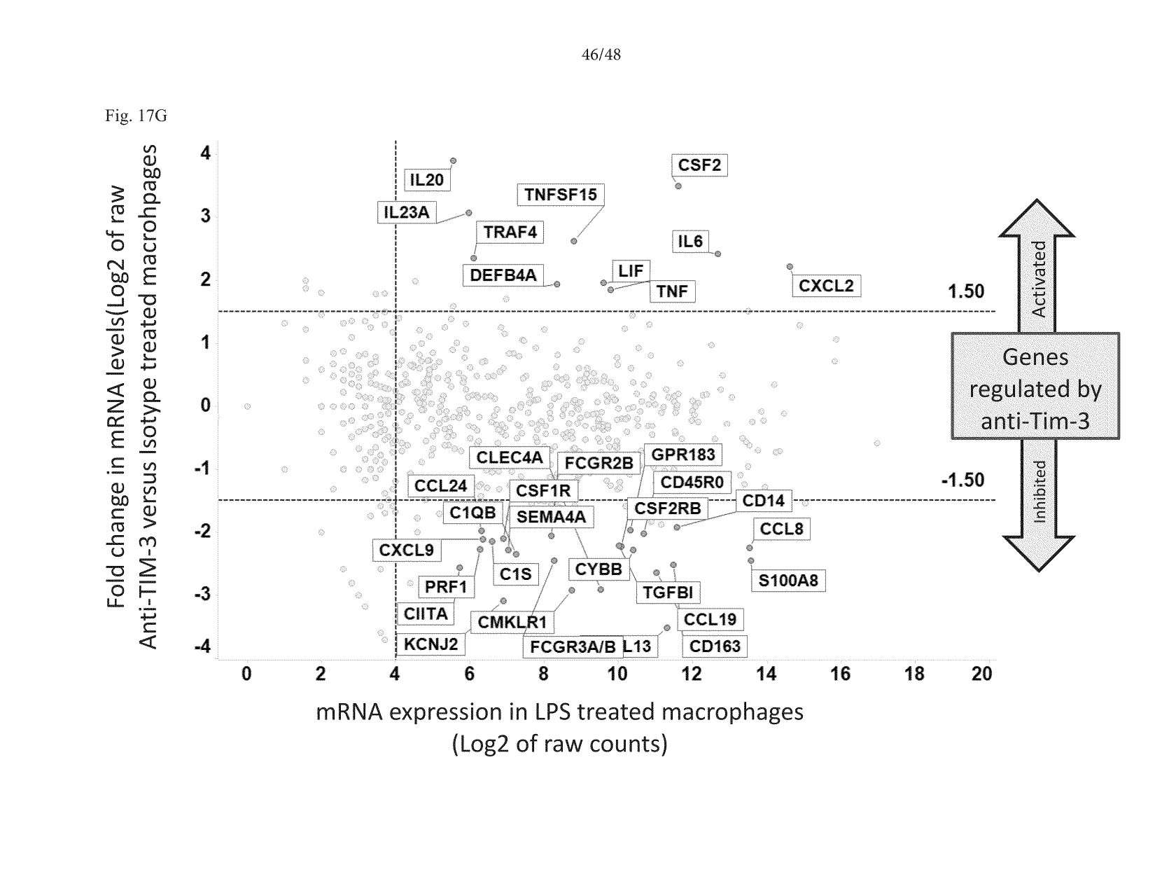

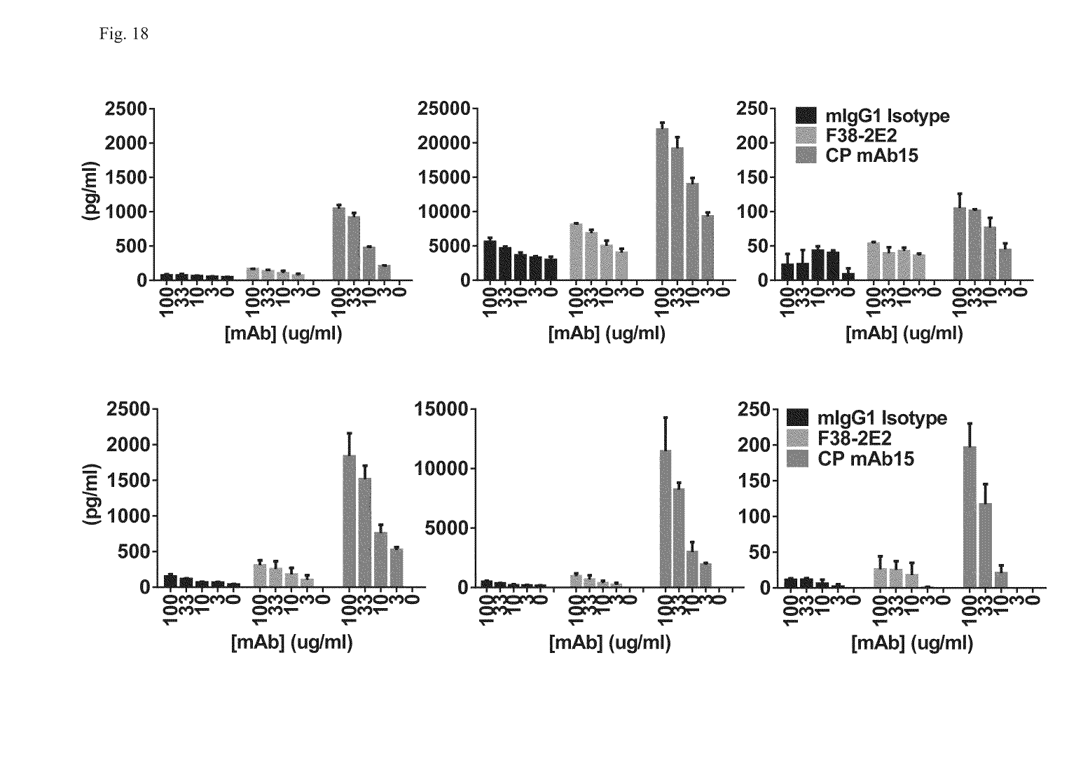

Cells were re-stimulated with PMA/Ionomycin on day 4 or day 7, cells were assessed for their expression of IL-1β, TNFα and IFN-γ by intracellular flow cytometry, and proliferation was measured by CFSE dilution. Results are shown in FIG. 18. mAb15 treatment stimulated expression of IL-1β, TNFα and IFN-γ by macrophages by day 1 post-treatment. This stimulation was greater than stimulation by antibody F38-2E2 for all three cytokines at both days 1 and 7. IL-1β was found to be generated mostly by CD14+ macrophages, both macrophages and T cells stained for TNFα and only CD8+ T cells stained for IFN-γ. Proliferation of CD8+ T cells was also increased by 10% by TIM-3 blockade with mAb15 compared to F38-2E2 and the isotype control. TIM-3 is not expressed by the T cells during the first few days of activation, so the blockade of TIM-3 on macrophages improves the function of T cells in this assay.

### Example 15

### Ovarian Cancer Responds to Anti-TIM-3 Blockade in Histoculture Assay

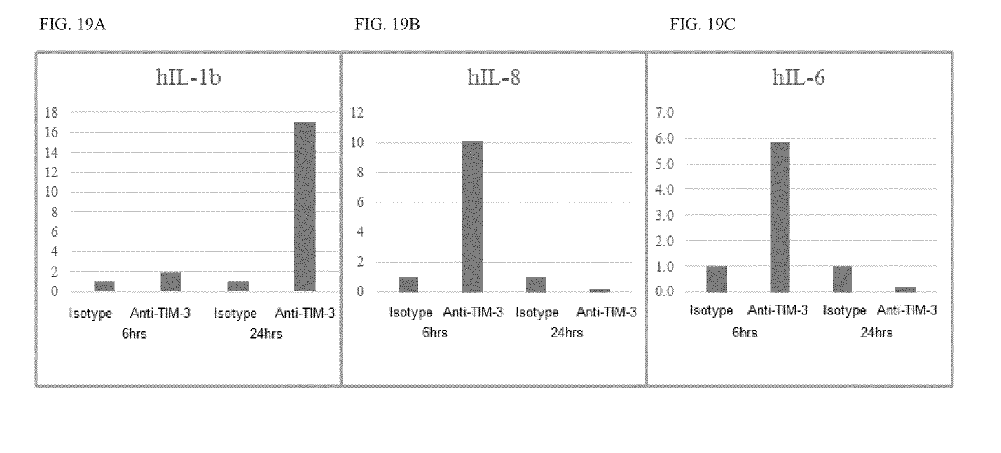

Primary ovarian tumor resections were obtained through the Cooperative Human Tissue Network (CHTN) or National Disease Research Interchange (NDRI). Samples were shipped in AQIX Solution (AQIX LTD, AQIX RS-I (10×), London, United Kingdom) within 24 hours of surgical resection. Upon arrival, a small piece of tumor was fixed with 4% paraformaldehyde and embedded in paraffin for later immunohistochemistry analyses. The remaining tumor was embedded in 4% agarose and sliced into 200-300 μM sections using a vibratome (Leica Biosystems, VT1000 S, Buffalo Grove, Ill.). Tissue slices were placed into 6-well plates on top of polycarbonate membrane inserts (ThermoFisher, ROCHESTER 140640, Grand Island, N.Y.) containing 1.5 mL of DMEM media supplemented with 8% FBS, 2% normal human serum (NHS), and 1× penicillin/streptomycin. In some wells anti-TIM-3 (mAb58) was added at 25 μg/mL and in other wells a negative control, Synagis hIgG4, was added at the same concentration. The tissue slices were incubated for 6 or 24 hours at 37° C. At the end of the culture period tissues were collected and RNA was extracted using the RNeasy Mini Kit (Qiagen, 74104, Gaithersburg, Md.). Quantitative real-time PCR was performed using TaqMan Probes (Applied BioSystems) against human IL-1β, IL-8, IL-6, GM-CSF, CD258, and IL-10. Data presented in FIG. 19 are representative of 2 independent experiments. Levels of IL-1β, IL-8 and IL-6 increased in response to anti-TIM-3 antibody compared to isotype control, with the greatest increase seen for IL-6 and IL-8 at 6 hours and for IL-1β at 24 hours post treatment. Similarly, levels of GM-CSF, CD258 and IL-10 also increased in response to anti-TIM-3 antibody, with the greatest increase observed at 6 hours post treatment.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

### Sequences

Human TIM-3 Isoform 1 amino acid sequence
(SEQ ID NO: 1)
MFSHLPFDCVLLLLLLLLTRSSEVEYRAEVGQNAYLPCFYTPAAPGNLVPVCWGKGACPVFECGNVVLRTDERDV
NYWTSRYWLNGDFRKGDVSLTIENVTLADSGIYCCRIQIPGIMNDEKFNLKLVIKPAKVTPAPTRQRDFTAAFPR
MLTTRGHGPAETQTLGSLPDINLTQISTLANELRDSRLANDLRDSGATIRIGIYIGAGICAGLALALIFGALIFK
WYSHSKEKIQNLSLISLANLPPSGLANAVAEGIRSEENIYTIEENVYEVEEPNEYYCYVSSRQQPSQPLGCRFAM
P
Human TIM-3 isoform 1 nucleic acid sequence
(SEQ ID NO: 2)
agaacactta caggatgtgt gtagtgtggc atgacagaga actttggttt cctttaatgt
gactgtagac ctggcagtgt tactataaga atcactggca atcagacacc cgggtgtgct
gagctagcac tcagtggggg cggctactgc tcatgtgatt gtggagtaga cagttggaag
aagtacccag tccatttgga gagttaaaac tgtgcctaac agaggtgtcc tctgactttt
cttctgcaag ctccatgttt tcacatcttc cctttgactg tgtcctgctg ctgctgctgc
tactacttac aaggtcctca gaagtggaat acagagcgga ggtcggtcag aatgcctatc
tgccctgctt ctacacccca gccgccccag ggaacctcgt gcccgtctgc tggggcaaag
gagcctgtcc tgtgtttgaa tgtggcaacg tggtgctcag gactgatgaa agggatgtga
attattggac atccagatac tggctaaatg gggatttccg caaaggagat gtgtccctga
ccatagagaa tgtgactcta gcagacagtg ggatctactg ctgccggatc caaatcccag
gcataatgaa tgatgaaaaa tttaacctga agttggtcat caaaccagcc aaggtcaccc
ctgcaccgac tcggcagaga gacttcactg cagcctttcc aaggatgctt accaccaggg
gacatggccc agcagagaca cagacactgg ggagcctccc tgatataaat ctaacacaaa
tatccacatt ggccaatgag ttacgggact ctagattggc caatgactta cgggactctg
gagcaaccat cagaataggc atctacatcg gagcagggat ctgtgctggg ctggctctgg
ctcttatctt cggcgcttta attttcaaat ggtattctca tagcaaagag aagatacaga
atttaagcct catctctttg gccaacctcc ctccctcagg attggcaaat gcagtagcag
agggaattcg ctcagaagaa aacatctata ccattgaaga gaacgtatat gaagtggagg
agcccaatga gtattattgc tatgtcagca gcaggcagca accctcacaa cctttgggtt
gtcgctttgc aatgccatag atccaaccac cttatttttg agcttggtgt tttgtctttt
tcagaaacta tgagctgtgt cacctgactg gttttggagg ttctgtccac tgctatggag
cagagttttc ccattttcag aagataatga ctcacatggg aattgaactg ggacctgcac
tgaacttaaa caggcatgtc attgcctctg tatttaagcc aacagagtta cccaacccag
agactgttaa tcatggatgt tagagctcaa acgggctttt atatacacta ggaattcttg
acgtggggtc tctggagctc caggaaattc gggcacatca tatgtccatg aaacttcaga
taaactaggg aaaactgggt gctgaggtga aagcataact tttttggcac agaaagtcta
aaggggccac tgattttcaa agagatctgt gatccctttt tgttttttgt ttttgagatg
gagtcttgct ctgttgccca ggctggagtg caatggcaca atctcggctc actgcaagct
ccgcctcctg ggttcaagcg attctcctgc ctcagcctcc tgagtggctg ggattacagg
catgcaccac catgcccagc taatttgttg tatttttagt agagacaggg tttcaccatg
ttggccagtg tggtctcaaa ctcctgacct catgatttgc ctgcctcggc ctcccaaagc
actgggatta caggcgtgag ccaccacatc cagccagtga tccttaaaag attaagagat
gactggacca ggtctacctt gatcttgaag attcccttgg aatgttgaga tttaggctta
tttgagcact gcctgcccaa ctgtcagtgc cagtgcatag cccttctttt gtctccctta
tgaagactgc cctgcagggc tgagatgtgg caggagctcc cagggaaaaa cgaagtgcat
ttgattggtg tgtattggcc aagttttgct tgttgtgtgc ttgaaagaaa atatctctga
ccaacttctg tattcgtgga ccaaactgaa gctatatttt tcacagaaga agaagcagtg
acggggacac aaattctgtt gcctggtgga aagaaggcaa aggccttcag caatctatat
taccagcgct ggatcctttg acagagagtg gtccctaaac ttaaatttca agacggtata
ggcttgatct gtcttgctta ttgttgcccc ctgcgcctag cacaattctg acacacaatt
ggaacttact aaaaattttt ttttactgtt aaaaaaaaaa aaaaaaaa
TIM-3 isoform 2 amino acid sequence
(SEQ ID NO: 3)
MFSHLPFDCVLLLLLLLLTRSSEVEYRAEVGQNAYLPCFYTPAAPGNLVPVCWGKGACPVFECGNVVLRTDERDV
NYWTSRYWLNGDFRKGDVSLTIENVTLADSGIYCCRIQIPGIMNDEKFNLKLVIKPGEWTFACHLYE
Human TIM-3 isoform 2 nucleic sequence
(SEQ ID NO: 4)
actgctcatg tgattgtgga gtagacagtt ggaagaagta cccagtccat ttggagagtt
aaaactgtgc ctaacagagg tgtcctctga cttttcttct gcaagctcca tgttttcaca
tcttcccttt gactgtgtcc tgctgctgct gctgctacta cttacaaggt cctcagaagt
ggaatacaga gcggaggtcg gtcagaatgc ctatctgccc tgcttctaca ccccagccgc
cccagggaac ctcgtgcccg tctgctgggg caaaggagcc tgtcctgtgt ttgaatgtgg
caacgtggtg ctcaggactg atgaaaggga tgtgaattat tggacatcca gatactggct
aaatggggat ttccgcaaag gagatgtgtc cctgaccata gagaatgtga ctctagcaga
cagtgggatc tactgctgcc ggatccaaat cccaggcata atgaatgatg aaaaatttaa
cctgaagttg gtcatcaaac caggtgagtg gacatttgca tgccatcttt atgaataaga
tttatctgtg gatcatatta aaggtactga ttgttctcat ctctgacttc cctaattata
gccctggagg agggccacta agacctaaag tttaacaggc cccattggtg atgctcagtg
atatttaaca ccttctctct gttttaaaac tcatgggtgt gcctgggcgt ggtggctcgc
gcctctggtc ccagcacttt gggaggctga ggccggtgga tcatgaggtc aggaattcga
gaccagcctg gccaacatgg taaaaccttg tctccactaa aaatacaaaa aattagccag
gcatggttac gggagcctgt aattctagct acttgggggg ctgaagcagg agaatcactt
gaacctggaa gtcggaggtt gcggtaagcc aagatctcgc cattgtactc cagcctggct
gacaagagtg aaactctgtc ccaaaaaaaa aaaaaaaaaa aaaaaaaaaa aa
LILRB2 variant 1 amino acid sequence
(SEQ ID NO: 5)
MTPIVTVLICLGLSLGPRTRVQTGTIPKPTLWAEPDSVITQGSPVTLSCQGSLEAQEYRLYREKKSASWITRIRP
ELVKNGQFHIPSITWEHTGRYGCQYYSRARWSELSDPLVLVMTGAYPKPTLSAQPSPVVTSGGRVTLQCESQVAF
GGFILCKEGEDEHPQCLNSQPHARGSSRAIFSVGPVSPNRRWSHRCYGYDLNSPYVWSSPSDLLELLVPGVSKKP
SLSVQPGPVMAPGESLTLQCVSDVGYDRFVLYKEGERDLRQLPGRQPQAGLSQANFTLGPVSRSYGGQYRCYGAH
NLSSECSAPSDPLDILITGQIRGTPFISVQPGPTVASGENVTLLCQSWRQFHTFLLTKAGAADAPLRLRSIHEYP
KYQAEFPMSPVTSAHAGTYRCYGSLNSDPYLLSHPSEPLELVVSGPSMGSSPPPTGPISTPAGPEDQPLTPTGSD
PQSGLGRHLGVVIGILVAVVLLLLLLLLLFLILRHRRQGKHWTSTQRKADFQHPAGAVGPEPTDRGLQWRSSPAA
DAQEENLYAAVKDTQPEDGVEMDTRAAASEAPQDVTYAQLHSLTLRRKATEPPPSQEREPPAEPSIYATLAIH
LILRB2 variant 1 nucleic acid sequence
(SEQ ID NO: 6)
atttggttga aagaaaaccc acaatccagt gtcaagaaag aagtcaactt ttcttcccct
acttccctgc atttctcctc tgtgctcact gccacacaca gctcaacctg gacagcacag
ccagaggcga gatgcttctc tgctgatctg agtctgcctg cagcatggac ctgggtcttc
cctgaagcat ctccagggct ggagggacga ctgccatgca ccgagggctc atccatccgc
agagcagggc agtgggagga gacgccatga cccccatcgt cacagtcctg atctgtctcg
ggctgagtct gggccccagg acccgcgtgc agacagggac catccccaag cccaccctgt
gggctgagcc agactctgtg atcacccagg ggagtcccgt caccctcagt tgtcagggga
gccttgaagc ccaggagtac cgtctatata gggagaaaaa atcagcatct tggattacac
ggatacgacc agagcttgtg aagaacggcc agttccacat cccatccatc acctgggaac
acacagggcg atatggctgt cagtattaca gccgcgctcg gtggtctgag ctcagtgacc
ccctggtgct ggtgatgaca ggagcctacc caaaacccac cctctcagcc cagcccagcc
ctgtggtgac ctcaggagga agggtgaccc tccagtgtga gtcacaggtg gcatttggcg
gcttcattct gtgtaaggaa ggagaagatg aacacccaca atgcctgaac tcccagcccc
atgcccgtgg gtcgtcccgc gccatcttct ccgtgggccc cgtgagcccg aatcgcaggt
ggtcgcacag gtgctatggt tatgacttga actctcccta tgtgtggtct tcacccagtg
atctcctgga gctcctggtc ccaggtgttt ctaagaagcc atcactctca gtgcagccgg
gtcctgtcat ggcccctggg gaaagcctga ccctccagtg tgtctctgat gtcggctatg
acagatttgt tctgtacaag gagggggaac gtgaccttcg ccagctccct ggccggcagc
cccaggctgg gctctcccag gccaacttca ccctgggccc tgtgagccgc tcctacgggg
gccagtacag atgctacggt gcacacaacc tctcctctga gtgctcggcc cccagcgacc
ccctggacat cctgatcaca ggacagatcc gtggcacacc cttcatctca gtgcagccag
gccccacagt ggcctcagga gagaacgtga ccctgctgtg tcagtcatgg cggcagttcc
acactttcct tctgaccaag gcgggagcag ctgatgcccc actccgtcta agatcaatac
acgaatatcc taagtaccag gctgaattcc ccatgagtcc tgtgacctca gcccacgcgg
ggacctacag gtgctacggc tcactcaact ccgaccccta cctgctgtct caccccagtg
agcccctgga gctcgtggtc tcaggaccct ccatgggttc cagcccccca cccaccggtc
ccatctccac acctgcaggc cctgaggacc agcccctcac ccccactggg tcggatcccc
aaagtggtct gggaaggcac ctgggggttg tgatcggcat cttggtggcc gtcgtcctac
tgctcctcct cctcctcctc ctcttcctca tcctccgaca tcgacgtcag ggcaaacact
ggacatcgac ccagagaaag gctgatttcc aacatcctgc aggggctgtg gggccagagc
ccacagacag aggcctgcag tggaggtcca gcccagctgc cgacgcccag gaagaaaacc
tctatgctgc cgtgaaggac acacagcctg aagatggggt ggagatggac actcgggctg
ctgcatctga agccccccag gatgtgacct acgcccagct gcacagcttg accctcagac
ggaaggcaac tgagcctcct ccatcccagg aaagggaacc tccagctgag cccagcatct
acgccaccct ggccatccac tagcccggag ggtacgcaga ctccacactc agtagaagga
gactcaggac tgctgaaggc acgggagctg cccccagtgg acaccaatga accccagtca
gcctggaccc ctaacaaaga ccatgaggag atgctgggaa ctttgggact cacttgattc
tgcagtcgaa ataactaata tccctacatt ttttaattaa agcaacagac ttctcaataa
tcaatgagtt aaccgagaaa actaaaatca gaagtaagaa tgtgctttaa actgaatcac
aatataaata ttacacatca cacaatgaaa ttgaaaaagt acaaaccaca aatgaaaaaa
gtagaaacga aaaaaaaaaa ctaggaaatg aatgacgttg gctttcgtat aaggaattta
gaaaaagaat aaccaattat tccaaatgaa ggtgtaagaa agggaataag aagaagaaga
gttgctcatg aggaaaaacc aaaacttgaa aattcaacaa agccaatgaa gctcattctt
gaaaatatta attacagtca taaatcctaa ctacattgag caagagaaag aaagagcagg
cacgcatttc catatgggag tgagccagca gacagcccag cagatcctac acacattttc
acaaactaac cccagaacag gctgcaaacc tataccaata tactagaaaa tgcagattaa
atggatgaaa tattcaaaac tggagtttac ataatgaacg taagagtaat cagagaatct
gactcatttt aaatgtgtgt gtatgtgtgt gtatatatat gtgtgtgtgt gtgtgtgtgt
gtgtgtgtga aaaacattga ctgtaataaa aatgttccca tcgtaaaaaa aaaaaaaaaa
LILRB2 variant 2 amino acid sequence
(SEQ ID NO: 7)
MTPIVTVLICLGLSLGPRTRVQTGTIPKPTLWAEPDSVITQGSPVTLSCQGSLEAQEYRLYREKKSASWITRIRP
ELVKNGQFHIPSITWEHTGRYGCQYYSRARWSELSDPLVLVMTGAYPKPTLSAQPSPVVTSGGRVTLQCESQVAF
GGFILCKEGEDEHPQCLNSQPHARGSSRAIFSVGPVSPNRRWSHRCYGYDLNSPYVWSSPSDLLELLVPGVSKKP
SLSVQPGPVMAPGESLTLQCVSDVGYDRFVLYKEGERDLRQLPGRQPQAGLSQANFTLGPVSRSYGGQYRCYGAH
NLSSECSAPSDPLDILITGQIRGTPFISVQPGPTVASGENVTLLCQSWRQFHTFLLTKAGAADAPLRLRSIHEYP
KYQAEFPMSPVTSAHAGTYRCYGSLNSDPYLLSHPSEPLELVVSGPSMGSSPPPTGPISTPGPEDQPLTPTGSDP
QSGLGRHLGVVIGILVAVVLLLLLLLLLFLILRHRRQGKHWTSTQRKADFQHPAGAVGPEPTDRGLQWRSSPAAD
AQEENLYAAVKDTQPEDGVEMDTRAAASEAPQDVTYAQLHSLTLRRKATEPPPSQEREPPAEPSIYATLAIH
LILRB2 variant 2 nucleic acid sequence
(SEQ ID NO: 8)
atttggttga aagaaaaccc acaatccagt gtcaagaaag aagtcaactt ttcttcccct
acttccctgc atttctcctc tgtgctcact gccacacaca gctcaacctg gacagcacag
ccagaggcga gatgcttctc tgctgatctg agtctgcctg cagcatggac ctgggtcttc
cctgaagcat ctccagggct ggagggacga ctgccatgca ccgagggctc atccatccgc
agagcagggc agtgggagga gacgccatga cccccatcgt cacagtcctg atctgtctcg
ggctgagtct gggccccagg acccgcgtgc agacagggac catccccaag cccaccctgt
gggctgagcc agactctgtg atcacccagg ggagtcccgt caccctcagt tgtcagggga
gccttgaagc ccaggagtac cgtctatata gggagaaaaa atcagcatct tggattacac
ggatacgacc agagcttgtg aagaacggcc agttccacat cccatccatc acctgggaac
acacagggcg atatggctgt cagtattaca gccgcgctcg gtggtctgag ctcagtgacc
ccctggtgct ggtgatgaca ggagcctacc caaaacccac cctctcagcc cagcccagcc
ctgtggtgac ctcaggagga agggtgaccc tccagtgtga gtcacaggtg gcatttggcg
gcttcattct gtgtaaggaa ggagaagatg aacacccaca atgcctgaac tcccagcccc
atgcccgtgg gtcgtcccgc gccatcttct ccgtgggccc cgtgagcccg aatcgcaggt
ggtcgcacag gtgctatggt tatgacttga actctcccta tgtgtggtct tcacccagtg
atctcctgga gctcctggtc ccaggtgttt ctaagaagcc atcactctca gtgcagccgg
gtcctgtcat ggcccctggg gaaagcctga ccctccagtg tgtctctgat gtcggctatg
acagatttgt tctgtacaag gagggggaac gtgaccttcg ccagctccct ggccggcagc
cccaggctgg gctctcccag gccaacttca ccctgggccc tgtgagccgc tcctacgggg
gccagtacag atgctacggt gcacacaacc tctcctctga gtgctcggcc cccagcgacc
ccctggacat cctgatcaca ggacagatcc gtggcacacc cttcatctca gtgcagccag
gccccacagt ggcctcagga gagaacgtga ccctgctgtg tcagtcatgg cggcagttcc
acactttcct tctgaccaag gcgggagcag ctgatgcccc actccgtcta agatcaatac
acgaatatcc taagtaccag gctgaattcc ccatgagtcc tgtgacctca gcccacgcgg
ggacctacag gtgctacggc tcactcaact ccgaccccta cctgctgtct caccccagtg
agcccctgga gctcgtggtc tcaggaccct ccatgggttc cagcccccca cccaccggtc
ccatctccac acctggccct gaggaccagc ccctcacccc cactgggtcg gatccccaaa
gtggtctggg aaggcacctg ggggttgtga tcggcatctt ggtggccgtc gtcctactgc
tcctcctcct cctcctcctc ttcctcatcc tccgacatcg acgtcagggc aaacactgga
catcgaccca gagaaaggct gatttccaac atcctgcagg ggctgtgggg ccagagccca
cagacagagg cctgcagtgg aggtccagcc cagctgccga cgcccaggaa gaaaacctct
atgctgccgt gaaggacaca cagcctgaag atggggtgga gatggacact cgggctgctg
catctgaagc cccccaggat gtgacctacg cccagctgca cagcttgacc ctcagacgga
aggcaactga gcctcctcca tcccaggaaa gggaacctcc agctgagccc agcatctacg
ccaccctggc catccactag cccggagggt acgcagactc cacactcagt agaaggagac
tcaggactgc tgaaggcacg ggagctgccc ccagtggaca ccaatgaacc ccagtcagcc
tggaccccta acaaagacca tgaggagatg ctgggaactt tgggactcac ttgattctgc
agtcgaaata actaatatcc ctacattttt taattaaagc aacagacttc tcaataatca
atgagttaac cgagaaaact aaaatcagaa gtaagaatgt gctttaaact gaatcacaat
ataaatatta cacatcacac aatgaaattg aaaaagtaca aaccacaaat gaaaaaagta
gaaacgaaaa aaaaaaacta ggaaatgaat gacgttggct ttcgtataag gaatttagaa
aaagaataac caattattcc aaatgaaggt gtaagaaagg gaataagaag aagaagagtt
gctcatgagg aaaaaccaaa acttgaaaat tcaacaaagc caatgaagct cattcttgaa
aatattaatt acagtcataa atcctaacta cattgagcaa gagaaagaaa gagcaggcac
gcatttccat atgggagtga gccagcagac agcccagcag atcctacaca cattttcaca
aactaacccc agaacaggct gcaaacctat accaatatac tagaaaatgc agattaaatg
gatgaaatat tcaaaactgg agtttacata atgaacgtaa gagtaatcag agaatctgac
tcattttaaa tgtgtgtgta tgtgtgtgta tatatatgtg tgtgtgtgtg tgtgtgtgtg
tgtgtgaaaa acattgactg taataaaaat gttcccatcg taaaaaaaaa aaaaaaa
Murine TIM-3 amino acid sequence
(SEQ ID NO: 9)
MFSGLTLNCVLLLLQLLLARSLENAYVFEVGKNAYLPCSYTLSTPGALVPMCWGKGFCPW
SQCTNELLRTDERNVTYQKSSRYQLKGDLNKGDVSLIIKNVTLDDHGTYCCRIQFPGLMN
DKKLELKLDIKAAKVTPAQTAHGDSTTASPRTLTTERNGSETQTLVTLHNNNGTKISTWA
DEIKDSGETIRTAIHIGVGVSAGLTLALIIGVLILKWYSCKKKKLSSLSLITLANLPPGG
LANAGAVRIRSEENIYTIEENVYEVENSNEYYCYVNSQQPS
Murine TIM-3 nucleic acid sequence
(SEQ ID NO: 10)
accattttaa ccgaggagct aaagctatcc ctacacagag ctgtccttgg atttcccctg
ccaagtactc atgttttcag gtcttaccct caactgtgtc ctgctgctgc tgcaactact
acttgcaagg tcattggaaa atgcttatgt gtttgaggtt ggtaagaatg cctatctgcc
ctgcagttac actctatcta cacctggggc acttgtgcct atgtgctggg gcaagggatt
ctgtccttgg tcacagtgta ccaacgagtt gctcagaact gatgaaagaa atgtgacata
tcagaaatcc agcagatacc agctaaaggg cgatctcaac aaaggagacg tgtctctgat
cataaagaat gtgactctgg atgaccatgg gacctactgc tgcaggatac agttccctgg
tcttatgaat gataaaaaat tagaactgaa attagacatc aaagcagcca aggtcactcc
agctcagact gcccatgggg actctactac agcttctcca agaaccctaa ccacggagag
aaatggttca gagacacaga cactggtgac cctccataat aacaatggaa caaaaatttc
cacatgggct gatgaaatta aggactctgg agaaacgatc agaactgcta tccacattgg
agtgggagtc tctgctgggt tgaccctggc acttatcatt ggtgtcttaa tccttaaatg
gtattcctgt aagaaaaaga agttatcgag tttgagcctt attacactgg ccaacttgcc
tccaggaggg ttggcaaatg caggagcagt caggattcgc tctgaggaaa atatctacac
catcgaggag aacgtatatg aagtggagaa ttcaaatgag tactactgct acgtcaacag
ccagcagcca tcctgaccgc ctctggactg ccacttttaa aggctcgcct tcatttctga
ctttggtatt tccctttttg aaaactatgt gatatgtcac ttggcaacct cattggaggt
tctgaccaca gccactgaga aaagagttcc agttttctgg ggataattaa ctcacaaggg
gattcgactg taactcatgc tacattgaaa tgctccattt tatccctgag tttcagggat
cggatctccc actccagaga cttcaatcat gcgtgttgaa gctcactcgt gctttcatac
attaggaatg gttagtgtga tgtctttgag acatagaggt ttgtggtata tctgcaaagc
tcctgaacag gtagggggaa taaagggcta agataggaag gtgaggttct ttgttgatgt
tgaaaatcta aagaagttgg tagcttttct agagatttct gaccttgaaa gattaagaaa
aagccaggtg gcatatgctt aacactatat aacttgggaa ccttaggcag gagggtgata
agttcaaggt cagccagggc tatgctggta agactgtctc aaaatccaaa gacgaaaata
aacatagaga cagcaggagg ctggagatga ggctcggaca gtgaggtgca ttttgtacaa
gcacgaggaa tctatatttg atcgtagacc ccacatgaaa aagctaggcc tggtagagca
tgcttgtaga ctcaagagat ggagaggtaa aggcacaaca gatccccggg gcttgcgtgc
agtcagctta gcctaggtgc tgagttccaa gtccacaaga gtccctgtct caaagtaaga
tggactgagt atctggcgaa tgtccatggg ggttgtcctc tgctctcaga agagacatgc
acatgaacct gcacacacac acacacacac acacacacac acacacacac acacacacac
acacacatga aatgaaggtt ctctctgtgc ctgctacctc tctataacat gtatctctac
aggactctcc tctgcctctg ttaagacatg agtgggagca tggcagagca gtccagtaat
taattccagc actcagaagg ctggagcaga agcgtggaga gttcaggagc actgtgccca
acactgccag actcttctta cagaagaaaa aggttacccg caagcagcct gctgtctgta
aaaggaaacc ctgcgaaagg caaactttga ctgttgtgtg ctcaagggga actgactcag
acaacttctc cattcctgga ggaaactgga gctgtttctg acagaagaac aaccggtgac
tgggacatac gaaggcagag ctcttgcagc aatctatata gtcagcaaaa tattctttgg
gaggacagtc gtcaccaaat tgatttccaa gccggtggac ctcagtttca tctggcttac
agctgcctgc ccagtgccct tgatctgtgc tggctcccat ctataacaga atcaaattaa
atagaccccg agtgaaaata ttaagtgagc agaaaggtag ctttgttcaa agattttttt
gcattgggga gcaactgtgt acatcagagg acatctgtta gtgaggacac caaaacctgt
ggtaccgttt tttcatgtat gaattttgtt gtttaggttg cttctagcta gctgtggagg
tcctggcttt cttaggtggg tatggaaggg agaccatcta acaaaatcca ttagagataa
cagctctcat gcagaaggga aaactaatct caaatgtttt aaagtaataa aactgtactg
gcaaagtact ttgagcatat ttaaa
mAb15 Heavy chain nucleic acid sequence
(SEQ ID NO: 11)
AAGCAGTGGTATCAACGCAGAGTACGCGGGGCATCATCTTCTCATAGAGCCTCCATCAGAGCATGGCTGTCCTGG
CATTACTCTTCTGCCTGGTAACATTCCCAAGCTGTATCCTTTCC
CAGGTGCAGCTGAAGGAGTCAGGACCTGGCCTGGTGGCGCCCTCACAGAGCCTGTCCATCACATGCACCGTCTCA
GGGTTCTCATTAACCGGCTATGGTGTTACCTGGGTTCGCCAGCCTCCAGGAAAGGGTCTGGAGTGGCTGGGAATG
ATATGGGGTGATGGAAACACAGACTATAATTCAGGTCTCAAATCCAGACTGAACATCAGCAAGGACAACTCCAAG
AGCCAAGTTTTCTTAAAAATGAACAGTCTGCAAACTGATGACACAGCCAGGTACTACTGTGCCAGGTCTTATTAC
TACGGTCCCCCTGACTACTGGGGCCAAGGCACCACTCTCACAGTCTCCTCAGCCAAAACGACACCCCCATCTGTC
TATCCACTGGCCCCTGGATCTGCTGCCCAAAACTAACTCCATGGTGACCCTGGGATGCCTGGTCAAGGGCTATTT
CCCTGAGCCAGTGACAGTGACCTGGAACTCTGGATCCCTGTCCAGCGGTGTGCACACCTTCC

Leader sequence is in italics
Body of the sequence is in CAPS
Start codon (ATG) in leader sequence is in italics and underlined
CDRs are underlined, according to the Kabat definition
CDRs are bolded, according to the IMGT definition

mAb15 variable heavy chain (VH) amino acid
sequence
(SEQ ID NO: 12)
MAVLALLFCLVTFPSCILSQVQLKESGPGLVAPSQSLSITCTVSGFSLTG
YGVTWVRQPPGKGLEWLGMIWGDGNTDYNSGLKSRLNISKDNSKSQV
FLKMNSLQTDDTARYYCARSYYYGPPDYWGQGTTLTVSS

CDRs are underlined, according to the Kabat definition
CDRS are bold, according to the IMGT definition

mAb15 light chain nucleic acid sequence

(SEQ ID NO: 13)

AAGCAGTGGTATCAACGCAGAGTACGCGGGGCATCAGACAGGCTGGGCAG
CAAGATGGAATCACAGACCCAGGTCCTCATGTTTCTTCTGCTCTGGGTAT
CTGGTGCCTGTGCAGACATTGTGATGACACAGTCTCCATCCTCCCTGGCT
ATGTCAGTAGGACAGAAGGTCACTATGAGCTGCAAGTCCAGTCAGAGCCT
TCTAAATAGTAGAAGTCAAAAGAACTATTTGGCCTGGTACCAGCGGAAAC
CAGGACAGTCTCCTAAACTTCTGCTATACTTTGCATCCACTAGGGAATCT
GGGGTCCCTGATCGCTTCATAGGCAGTGGATCTGGGACAGATTTCACTC
TTACCATCAGCAGTGTGCAGGCTGAAGACCTGGCAGATTACTTCTGTCAC
CAACATTATAACACTCCGTACACGTTCGGAGGGGGGACCAAGCTGGAAA
TTAAACGGGCTGATGCTGCACCAACTGTATCCATCTTCCCACCATCCAGT
GAGCAGTTA

Leader sequence is in italics
Body of the sequence is in CAPS
Start codon (ATG) in leader sequence is in italics and underlined
CDRs according to Kabat definition are underlined

mAb15 variable light chain (VL) nucleic
acid sequence

(SEQ ID NO: 14)

MESQTQVLMFLLLWVSGACADIVMTQSPSSLAMSVGQKVTMSCKSSQSL
LNSRSQKNYLAWYQRKPGQSPKLLLYFASTRESGVPDRFIGSGSGTDFTL
TISSVQAEDLADYFCHQHYNTPYTFGGGTKLEIK

CDRs are underlined, according to the Kabat definition
CDRs are bold, according to the IMGTdefinition

mAb15 heavy chain CDRs-Kabat

(SEQ ID NO: 59)

GYGVT

(SEQ ID NO: 80)

MIWGDGNTDYNSGLKS

(SEQ ID NO: 81)

SYYYGPPDY
mAb15 heavy chain CDRs-IMGT

(SEQ ID NO: 15)

GFSLTGYG

(SEQ ID NO: 16)

IWGDGNT

(SEQ ID NO: 17)

ARSYYYGPPDY
mAb15 light chain CDRs-Kabat

(SEQ ID NO: 88)

KSSQSLLNSRSQKNYLA

(SEQ ID NO: 89)

FASTRES

(SEQ ID NO: 20)

HQHYNTPYT
mAb15 light chain CDRs-IMGT

(SEQ ID NO: 18)

QSLLNSRSQKNY

(SEQ ID NO: 19)

FAS

(SEQ ID NO: 20)

HQHYNTPYT
mAb13 VH

(SEQ ID NO: 21)

QIQLQQSGPELVKPGTSVKISCKASGYTFTDYYINWVKQRPGQGLEWIG
WIYPGSGNTKYNEKFKGKATLTVDTSSSTAYMQLSSLTSEDSAVYFFAR
GGKYYAMDYWGQGTSVIVSS

CDRs are underlined, according to Kabat.
CDRs are bolded according to ABM

mAb13 VL

(SEQ ID NO: 22)

NIVMTQTPKFLLISAGDRVTITCKASQSVGNNVAWYQQKPGQSPKLLIY
YASNRYTGVPDRFTGSAYGTDFTFTITTVQAEDLAVYFCQQDYSSPYTF
GGGTKLEIK

CDRs are underlined, according to Kabat.
CDRs are bolded according to ABM

mAb13 heavy chain CDRs-Kabat

(SEQ ID NO: 90)

DYYIN

(SEQ ID NO: 28)

WIYPGSGNTKYNEKFKG

(SEQ ID NO: 29)

GGKYYAMDY
mAb13 heavy chain CDRs-ABM

(SEQ ID NO: 27)

GYTFTDYYIN

(SEQ ID NO: 96)

WIYPGSGNTK

(SEQ ID NO: 29)

GGKYYAMDY
mAb13 light chain CDRs-Kabat and ABM

(SEQ ID NO: 30)

KASQSVGNNVA

(SEQ ID NO: 31)

YASNRYT

(SEQ ID NO: 32)

QQDYSSPYT
mAb17 VH

(SEQ ID NO: 23)

EVQLVESGGDLVKPGGSLKLSCAASGFTFSNYGMSWVRQTPDKRLEWVAT
ISSGGSNTYFPDSVKGRFTISRDNAKNTLYLQMSSLKSEDTAMYYCARHG
TSMIKEWFAYWGQGTLVTVSA

CDRs are underlined, according to Kabat.
CDRs are bolded, according to ABM

mAb17 VL

(SEQ ID NO: 24)

DIVMTQSPVTLSVTPGDRVSLSCRASQSIGDYLHWYQQKSHESPRLLI
KYASQSISGIPSRFSGSGSGSDFTLNINSVEPEDVGVYYCQNSHSFPP
TFGGGTRLEIK

CDRs are underlined, according to Kabat.
CDRs are bolded, according to ABM

mAb17 heavy chain CDRs-Kabat

(SEQ ID NO: 91)

NYGMS

(SEQ ID NO: 34)

TISSGGSNTYFPDSVKG

(SEQ ID NO: 35)

HGTSMIKEWFAY
mAb17 heavy chain CDRs-ABM

(SEQ ID NO: 33)

GFTFSNYGMS

(SEQ ID NO: 98)

TISSGGSNTY

(SEQ ID NO: 35)

HGTSMIKEWFAY
mAb17 light chain CDRs-Kabat and ABM

(SEQ ID NO: 36)

RASQSIGDYLH

(SEQ ID NO: 37)

YASQSIS

(SEQ ID NO: 38)

QNSHSFPPT
mAb22 VH

(SEQ ID NO: 25)

EVQLVESGGDLVKPGGSLKLSCAASGFTFSNHGMSWVRQTPDKRLEWVA
TISSGGSNTYFPDSVKGRFTISRDNVKNSLYLQMSSLKSEDTAMYYCARH
GTSMIKEWFAYWGQGTLVTVSA

CDRs are underlined, according to Kabat.
CDRs are bolded, according to ABM

mAb22 VL

(SEQ ID NO: 26)

DIVMTQSPVTLSVTPGDRVSLSCRASQSIGDYLHWYQQKSHESPRLLI
KYASQSISGIPSRFSGSGSGSDFTLNINSVEPEDVGVYYCQHSHSFPP
TFGGGTRLEIK

CDRs are underlined, according to Kabat.
CDRs are bolded, according to ABM

mAb22 heavy chain CDRs-Kabat

(SEQ ID NO: 97)

NHGMS

(SEQ ID NO: 34)

TISSGGSNTYFPDSVKG

(SEQ ID NO: 35)

HGTSMIKEWFAY
mAb22 heavy chain CDRs-ABM

(SEQ ID NO: 39)

GFTFSNHGMS

(SEQ ID NO: 98)

TISSGGSNTY

(SEQ ID NO: 35)

HGTSMIKEWFAY
mAb22 light chain CDRs-Kabat and ABM

(SEQ ID NO: 36)

RASQSIGDYLH

(SEQ ID NO: 37)

YASQSIS

(SEQ ID NO: 40)

QHSHSFPPT

mAb58 sequences

| SEQ | Seq | | | |
|---|---|---|---|---|
| ID | Name | Origin | Region | Sequence |
| 41 | VH0 | hybridoma | VH | QIQLVQSGPELKKPGETVKISCKASGYTFTTYGMSWVKQ |
| | | (murine) | | APGKGLKLMGWINTYSGAPTYADDFKGRFAFSLETSASA |
| | | | | AYLQINNLKNEDTATYFCARKPPHYYVNSFDYWGQGTTL |
| | | | | TVSS |
| 42 | VH1 | humanized | VH | QVQLVQSGSELKKPGAPVKVSCKASGYTFTTYGMSWVRQ |
| | | | | APGQGLEWMGWINTYSGAPTYADDFKGRFVFSLDTSVST |
| | | | | AYLQISSLKAEDTAVYYCARKPPHYYVNSFDYWGQGTTV |
| | | | | TVSS |
| 43 | VH2 | humanized | VH | QVHLVQSGSELKKPGASVKISCKASGYTFTTYGMSWVRQ |
| | | | | APGQGLEWMGWINTYSGAPTYADDFKGRFVFSLDTSVTT |
| | | | | SYLQISTLKAEDTAVYFCARKPPHYYVNSFDYWGQGTLV |
| | | | | TVSS |
| 44 | VH3 | humanized | VH | QVQLVQSGSELKKPGASVKISCKASGYTFTTYGMSWMRQ |
| | | | | APGQGLKWMGWINTYSGAPTYADDFKGRFVFSLDTSVNT |
| | | | | AFLQISSLQAEDTAVYYCARKPPHYYVNSFDYWGQGTTV |
| | | | | TVSS |
| 45 | VH4 | humanized | VH | QVQLVQSGPEVKKPGASVKVSCKTSGYTFTTYGMSWVRQ |
| | | | | APGQGLEWMGWINTYSGAPTYADDFKGRVTMTTDTSTST |
| | | | | AYLELTGLMSDDTAVYFCARKPPHYYVNSFDYWGQGTTV |
| | | | | TVSS |
| 46 | VH5 | humanized | VH | QVQLVQSGPEVKKPGASVKVSCKASGYTFTTYGMSWVRQ |
| | | | | APGQGLEWMGWINTYSGAPTYADDFKGRVTITRDTPTSA |
| | | | | VYLDLRSLRSDDTAVYYCARKPPHYYVNSFDYWGQGTLV |
| | | | | TVSS |
| 47 | VH6 | humanized | VH | QVQLVQSGAEVKKPGASVKVSCKASGYTFTTYGMSWVRQ |
| | | | | APGQRLEWMGWINTYSGAPTYADDFKGRVTITRDTSAST |
| | | | | AYMELSSLRSEDTAVYYCARKPPHYYVNSFDYWGQGTLV |
| | | | | TVSS |
| 48 | VL0 | hybridoma | VL | DIVMTQSPATLSVTPGDRVSLSCRASQSISDYLHWYQQK |
| | | (murine) | | SHESPRLLIKYASQSISGIPSRFSGSGSGSDFTLSINSV |
| | | | | EPEDVGVYYCQNGHSFPYTFGGGTKLEIK |
| 49 | VL1 | humanized | VL | EIVLTQSPATLSLSPGERATLSCRASQSISDYLHWYQQK |
| | | | | PGQAPRLLIHYASQSISGIPARFSGSGSGTDFTLTISSL |
| | | | | EPEDFAVYYCQNGHSFPYTFGGGTKVEIK |
| 50 | VL2 | humanized | VL | EIVLTQSPDFQSVTPKEKVTITCRASQSISDYLHWYQQK |
| | | | | PDQSPKLLIKYASQSISGVPSRFSGSGSGTDFTLTINSL |
| | | | | EAEDAATYYCQNGHSFPYTFGQGTKVEIK |
| 51 | VL3 | humanized | VL | DIVMTQSPSSLSASVGDRVTITCRASQSISDYLHWYQQK |
| | | | | PGKAPKLLIYYASQSISGVPSRFSGSGSGTDFTLTISSL |
| | | | | QPEDFATYYCQNGHSFPYTFGGGTKVEIK |
| 52 | VL4 | humanized | VL | EIVMTQSPATLSVSPGERATLSCRASQSISDYLHWYQQK |
| | | | | PGEAPRLLIYYASQSISGIPARFSGSGSGTDFTLTISSL |
| | | | | EPEDFAVYYCQNGHSFPYTFGQGTKLEIK |

### mAb 58 CDRs

The CDRs were identified according to the Kabat definition, and are highlighted in bold and underlined below.

| SEQ | Seq | | | |
|---|---|---|---|---|
| ID | Name | Origin | Region | Sequence |
| 41 | VH0 | hybridoma | VH | QIQLVQSGPELKKPGETVKISCKASGYTFTTYGMSWVKQAPG |
| | | (murine) | | KGLKLMGWINTYSGAPTYADDFKGRFAFSLETSASAAYLQIN |
| | | | | NLKNEDTATYFCARKPPHYYVNSFDYWGQGTTLTVSS |
| 48 | VL0 | hybridoma | VL | DIVMTQSPATLSVTPGDRVSLSCRASQSISDYLHWYQQKSHE |
| | | (murine) | | SPRLLIKYASQSISGIPSRFSGSGSGSDFTLSINSVEPEDVG |
| | | | | VYYCQNGHSFPYTFGGGTKLEIK |

| SEQ | Seq | |
|---|---|---|
| ID | Name | Sequence |
| 55 | CDR H1 | TYGMS |
| 56 | CDR H2 | WINTYSGAPTYADDFKG |
| 57 | CDR H3 | KPPHYYVNSFDY |
| 58 | CDR L1 | RASQSISDYLH |
| 37 | CDR L2 | YASQSIS |
| 60 | CDR L3 | QNGHSFPYT |

### mAb 58 Constant Region Sequences

| SEQ | Seq | | |
|---|---|---|---|
| ID | Name | Origin | Sequence |
| 61 | hIgG4 | human | ASTKGPSVFPLAPCSRSTSESTAALGCLVKDYFPEPVTVSWN |
| | S228P | | SGALTSGVHTFPAVLQSSGLYSLSSVVTVPSSSLGTKTYTCN |
| | | | VDHKPSNTKVDKRVESKYGPPCPPCPAPEFLGGPSVFLFPPK |
| | | | PKDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVYTLPPSQEEMTKNQVSLTCLVKGF |
| | | | YPSDIAVEWESNGQPENNYKTTPPVLDSDGSFFLYSRLTVDK |
| | | | SRWQEGNVFSCSVMHEALHNHYTQKSLSLSLGK |
| 62 | hKappa | human | RTVAAPSVFIFPPSDEQLKSGTASVVCLLNNFYPREAKVQWK |
| | | | VDNALQSGNSQESVTEQDSKDSTYSLSSTLTLSKADYEKHKV |
| | | | YACEVTHQGLSSPVTKSFNRGEC |

### Loop Swap Experiment

The table below has the sequences used for generating TIM3-Fc chimera proteins, used for TIM3 antibody epitope mapping. Within the ‘Sequence’ column cell, the first block of amino acids is the sequence of the hTIM3 ECD region used in the construct, and the second block of amino acids is a short linker followed by the human IgG1 Fc (this linker-Fc region is the same for all constructs).

### Loop Chimera Sequences

| SEQ | | Loop | |
|---|---|---|---|
| ID | Construct | Chimera | Sequence |
| 63 | hTIM3- | WT | SEVEYRAEVGQNAYLPCFYTPAAPGNLVPVCWGKGACPVFEC |
| | Fc | | GNVVLRTDERDVNYWTSRYWLNGDFRKGDVSLTIENVTLADS |
| | | | GIYCCRIQIPGIMNDEKFNLKLVIKPAKVTPAPTRQRDFTAA |
| | | | FPRMLTTRGHGPAETQTLGSLPDINLTQISTLANELRDSRLA |
| | | | NDLRDSGATIR |
| | | | IEGRMDPKSCDKTHTCPPCPAPELLGGPSVFLFPPKPKDTLM |
| | | | ISRTPEVTCVVVDVSHEDPEVKFNWYVDGVEVHNAKTKPREE |
| | | | QYNSTYRVVSVLTVLHQDWLNGKEYKCKVSNKALPAPIEKTI |
| | | | SKAKGQPREPQVYTLPPSREEMTKNQVSLTCLVKGFYPSDIA |
| | | | VEWESNGQPENNYKTTPPVLDSDGSFFLYSKLTVDKSRWQQG |
| | | | NVFSCSVMHEALHNHYTQKSLSLSPGK |
| 64 | hTIM3- | BC | SEVEYRAEVGQNAYLPCFYTLPTSGTLVPVCWGKGACPVFEC |
| | Fc | | GNVVLRTDERDVNYWTSRYWLNGDFRKGDVSLTIENVTLADS |
| | | | GIYCCRIQIPGIMNDEKFNLKLVIKPAKVTPAPTRQRDFTAA |
| | | | FPRMLTTRGHGPAETQTLGSLPDINLTQISTLANELRDSRLA |
| | | | NDLRDSGATIR |
| | | | IEGRMDPKSCDKTHTCPPCPAPELLGGPSVFLFPPKPKDTLM |
| | | | ISRTPEVTCVVVDVSHEDPEVKFNWYVDGVEVHNAKTKPREE |
| | | | QYNSTYRVVSVLTVLHQDWLNGKEYKCKVSNKALPAPIEKTI |
| | | | SKAKGQPREPQVYTLPPSREEMTKNQVSLTCLVKGFYPSDIA |
| | | | VEWESNGQPENNYKTTPPVLDSDGSFFLYSKLTVDKSRWQQG |
| | | | NVFSCSVMHEALHNHYTQKSLSLSPGK |
| 65 | hTIM3- | CC′ | SEVEYRAEVGQNAYLPCFYTPAAPGNLVPVCWGKGACPWSQC |
| | Fc | | GNVVLRTDERDVNYWTSRYWLNGDFRKGDVSLTIENVTLADS |
| | | | GIYCCRIQIPGIMNDEKFNLKLVIKPAKVTPAPTRQRDFTAA |
| | | | FPRMLTTRGHGPAETQTLGSLPDINLTQISTLANELRDSRLA |
| | | | NDLRDSGATIR |
| | | | IEGRMDPKSCDKTHTCPPCPAPELLGGPSVFLFPPKPKDTLM |
| | | | ISRTPEVTCVVVDVSHEDPEVKFNWYVDGVEVHNAKTKPREE |
| | | | QYNSTYRVVSVLTVLHQDWLNGKEYKCKVSNKALPAPIEKTI |
| | | | SKAKGQPREPQVYTLPPSREEMTKNQVSLTCLVKGFYPSDIA |
| | | | VEWESNGQPENNYKTTPPVLDSDGSFFLYSKLTVDKSRWQQG |
| | | | NVFSCSVMHEALHNHYTQKSLSLSPGK |
| 66 | hTIM3- | C′C″ | SEVEYRAEVGQNAYLPCFYTPAAPGNLVPVCWGKGACPVFEC |
| | Fc | | GNVVLRTDERNVTYWTSRYWLNGDFRKGDVSLTIENVTLADS |
| | | | GIYCCRIQIPGIMNDEKFNLKLVIKPAKVTPAPTRQRDFTAA |
| | | | FPRMLTTRGHGPAETQTLGSLPDINLTQISTLANELRDSRLA |
| | | | NDLRDSGATIR |
| | | | IEGRMDPKSCDKTHTCPPCPAPELLGGPSVFLFPPKPKDTLM |
| | | | ISRTPEVTCVVVDVSHEDPEVKFNWYVDGVEVHNAKTKPREE |
| | | | QYNSTYRVVSVLTVLHQDWLNGKEYKCKVSNKALPAPIEKTI |
| | | | SKAKGQPREPQVYTLPPSREEMTKNQVSLTCLVKGFYPSDIA |
| | | | VEWESNGQPENNYKTTPPVLDSDGSFFLYSKLTVDKSRWQQG |
| | | | NVFSCSVMHEALHNHYTQKSLSLSPGK |
| 67 | hTIM3- | DE | SEVEYRAEVGQNAYLPCFYTPAAPGNLVPVCWGKGACPVFEC |
| | Fc | | GNVVLRTDERDVNYWTSRYWLNGDLNKGDVSLTIENVTLADS |
| | | | GIYCCRIQIPGIMNDEKFNLKLVIKPAKVTPAPTRQRDFTAA |
| | | | FPRMLTTRGHGPAETQTLGSLPDINLTQISTLANELRDSRLA |
| | | | NDLRDSGATIR |
| | | | IEGRMDPKSCDKTHTCPPCPAPELLGGPSVFLFPPKPKDTLM |
| | | | ISRTPEVTCVVVDVSHEDPEVKFNWYVDGVEVHNAKTKPREE |
| | | | QYNSTYRVVSVLTVLHQDWLNGKEYKCKVSNKALPAPIEKTI |
| | | | SKAKGQPREPQVYTLPPSREEMTKNQVSLTCLVKGFYPSDIA |
| | | | VEWESNGQPENNYKTTPPVLDSDGSFFLYSKLTVDKSRWQQG |
| | | | NVFSCSVMHEALHNHYTQKSLSLSPGK |
| 68 | hTIM3- | FG | SEVEYRAEVGQNAYLPCFYTPAAPGNLVPVCWGKGACPVFEC |
| | Fc | | GNVVLRTDERDVNYWTSRYWLNGDFRKGDVSLTIENVTLADS |
| | | | GIYCCRIQFPGLMNDKKFNLKLVIKPAKVTPAPTRQRDFTAA |
| | | | FPRMLTTRGHGPAETQTLGSLPDINLTQISTLANELRDSRLA |
| | | | NDLRDSGATIR |
| | | | IEGRMDPKSCDKTHTCPPCPAPELLGGPSVFLFPPKPKDTLM |
| | | | ISRTPEVTCVVVDVSHEDPEVKFNWYVDGVEVHNAKTKPREE |
| | | | QYNSTYRVVSVLTVLHQDWLNGKEYKCKVSNKALPAPIEKTI |
| | | | SKAKGQPREPQVYTLPPSREEMTKNQVSLTCLVKGFYPSDIA |
| | | | VEWESNGQPENNYKTTPPVLDSDGSFFLYSKLTVDKSRWQQG |
| | | | NVFSCSVMHEALHNHYTQKSLSLSPGK |
| 69 | hTIM3- | IgV | SEVEYRAEVGQNAYLPCFYTPAAPGNLVPVCWGKGACPVFEC |
| | Fc | | GNVVLRTDERDVNYWTSRYWLNGDFRKGDVSLTIENVTLADS |
| | | | GIYCCRIQIPGIMNDEKFNLKLVIK |
| | | | IEGRMDPKSCDKTHTCPPCPAPELLGGPSVFLFPPKPKDTLM |
| | | | ISRTPEVTCVVVDVSHEDPEVKFNWYVDGVEVHNAKTKPREE |
| | | | QYNSTYRVVSVLTVLHQDWLNGKEYKCKVSNKALPAPIEKTI |
| | | | SKAKGQPREPQVYTLPPSREEMTKNQVSLTCLVKGFYPSDIA |
| | | | VEWESNGQPENNYKTTPPVLDSDGSFFLYSKLTVDKSRWQQG |
| | | | NVFSCSVMHEALHNHYTQKSLSLSPGK |

### Additional mAb V Region Sequences

CDRs are identified according to the Kabat definition, and underlined below.

| SEQ | Seq | Re- | |
|---|---|---|---|
| ID | Name | gion | Sequence |
| 70 | mAb48 | VH | QIQLQQSGPELVTPGTSVKISCKASGYTFTDYYIN |
| | | | WVKQRPGQGLEWIGWIYPGSGNTKYNEKFKGKATL |
| | | | TVDTSSSIAYMHLSSLTSEDSAVYFCAGDRFDYWG |
| | | | QGTTLTVSS |
| 71 | mAb48 | VL | QIVLTQSPAIMSASPGEKVTLTCSASSGVSSSYLY |
| | | | WYQQKPGSSPKLWIYSTSNLASGVPARFSGSGSGT |
| | | | SYSLTISNMETEDAASYFCHQWSNSPYTFGGGTKL |
| | | | EIK |
| 72 | mAb91 | VH | DVQFQESGPGLVKPSQSLSLTCSVTGYSITSGYYW |
| | | | NWIRQFPGNKLEWMGYISYDGSNNYNPSLKNRISI |
| | | | TRDTSKNQFFLKLNSVTTEDTATYYCGRDGPYYYG |
| | | | SSYGYFDVWGTGTTVTVSS |
| 73 | mAb91 | VL | DIVMTQAESSVPVTPGESVSISCRSSKSLLHSNGN |
| | | | TYLYWFLQRPGQSPQLLIHRMSNLASGVPDRFSGS |
| | | | GSGTAFTLRISRVEAEDMGVYYCMQHLEYPCTFGG |
| | | | GTKLEIK |
| 53 | mAb92 | VH | QVQLQQSGAELARPGASVKLSCKASGYSFTNYGIS |
| | | | WVKQRTGQGLEWIGEIYLRSGNNYYNEKFKGKATL |
| | | | TGDKSSSTAYMELRSLTSEDSAVYFCAIYGNYFYF |
| | | | DYWGQGTTLTVSS |
| 54 | mAb92 | VL | DIQMTQSPSSLSASLGERVSLTCRASQDIGSSLNW |
| | | | LQQEPDGTIKRLIYATSSLDSGVPKRFSGSRSGSD |
| | | | YSLTISSLESEDFVDYYCLQYASSPFTFGSGTKLE |
| | | | IK |

1: An antibody which inhibits the interaction of TIM-3 and LILRB2.

2: The antibody of claim 1 which inhibits the binding of TIM-3 to LILRB2.

3: An antibody which specifically binds TIM-3, wherein binding of the antibody to TIM-3 inhibits the interaction of TIM-3 to LILRB2.

4: The antibody of claim 3, wherein binding of the antibody to TIM-3 inhibits binding of TIM-3 to LILRB2.

5: The antibody of claim 3, wherein the antibody competes with LILRB2 for binding to TIM-3.

6: The antibody of claim 1 or 3, wherein the TIM-3 is human TIM-3.

7: The antibody of claim 1 or 3, wherein the TIM-3 comprises the amino acid sequence of SEQ ID NO:1 or SEQ ID NO:3.

8: The antibody of claim 1 or 3, wherein the amino acid sequence of the TIM-3 is at least about 80% identical to the amino acid sequence set forth in SEQ ID NO:1 or SEQ ID NO:3.

9: The antibody of claim 1 or 3, wherein the LILRB2 is human LILRB2.

10: The antibody of claim 1 or 3, wherein the LILRB2 comprises the amino acid sequence of SEQ ID NO:5 or SEQ ID NO:7.

11: The antibody of claim 1 or 3, wherein the amino acid sequence of the LILRB2 is at least about 80% identical to the amino acid sequence set forth in SEQ ID NO:5 or SEQ ID NO:7.

12: An antibody which specifically binds LILRB2, wherein binding of the antibody to LILRB2 inhibits the interaction of LILRB2 to TIM-3.

13-14. (canceled)

15: The antibody of claim 1 or 3, wherein the antibody competes with antibody mAb13, mAb15, mAb17, mAb22, mAb48, mAb58 and/or mAb91 for binding human TIM-3.

16: The antibody of claim 1 or 3, wherein the antibody stimulates the secretion of one or more myeloid-associated cytokines in an individual.

17: The antibody of claim 16, wherein the myeloid associated cytokine one or more of IL-12, TNFα, IL-1β, GM-CSF or IL-6.

18: The antibody of claim 16, wherein the antibody stimulates the secretion of one or more myeloid-associated cytokines in an individual to a greater extent than the stimulation of secretion of the cytokine by antibody F38-2E2.

19: The antibody of claim 1 or 3, wherein the antibody suppresses the secretion of a myeloid-associated cytokine in an individual.

20: The antibody of claim 19, wherein the myeloid associated cytokine is IL-10, CCL2, CCL3, CCL4 or CCL5.

21: The antibody of claim 19, wherein the antibody suppresses the secretion of a myeloid-associated cytokine in an individual to a greater extent than the suppression of secretion of the cytokine by antibody F38-2E2.

22: An antibody the binds TIM-3, wherein the antibody stimulates the secretion of one or more myeloid-associated cytokines in an individual.

23.-31. (canceled)

32: The antibody of claim 1 or 3, wherein the antibody is a monoclonal antibody.

33: The antibody of claim 1 or 3, wherein the antibody is a chimeric antibody, a humanized antibody or a human antibody.

34-35. (canceled)

36: The antibody of claim 1 or 3, wherein the antibody is an antibody fragment selected from a Fab, Fab′, Fv, scFv or (Fab′)2 fragment.

37: A pharmaceutical composition comprising the antibody of claim 1 or 3 and a pharmaceutically acceptable carrier.

38: A method of modulating the secretion of a myeloid-associated cytokine in an individual, comprising administering to the individual a therapeutically effective amount of the antibody of claim 1 or 3.

39-48. (canceled)

49: A method for treating cancer in an individual, comprising administering to the individual a therapeutically effective amount of the antibody of claim 1 or 3.

50. (canceled)

51: An isolated nucleic acid encoding the antibody of claim 1 or 3.

52. (canceled)

53: A host cell comprising the nucleic acid of claim 51.

54: A host cell that produces the antibody of claim 1 or 3.

55: A method for making an antibody that inhibits the interaction of TIM-3 and LILRB2, the method comprising culturing the host cell of claim 54 under conditions suitable for expression of the nucleic acid encoding the antibody that inhibits the interaction of TIM-3 and LILRB2.

56: An isolated nucleic acid encoding the antibody of claim 22.

57-58. (canceled)

59: A host cell that produces the antibody of claim 22.

60: A method for making an antibody that binds TIM-3 and stimulates secretion of one or more myeloid-associated cytokines, the method comprising culturing the host cell of claim 59 under conditions suitable for expression of the nucleic acid encoding the antibody.

61-73. (canceled)

74: A pharmaceutical composition for treating cancer in an individual comprising a therapeutically effective amount of an antibody that inhibits the interaction of TIM-3 and LILRB2 of claim 1 or 3 and a pharmaceutically acceptable carrier.

75: A kit for modulating of a myeloid-associated cytokine in an individual, comprising the antibody of claim 1 or 3.

76-84. (canceled)

85: A method for screening an agent which inhibits the interaction of TIM-3 and LILRB2, the method comprising measuring the binding of TIM-3 and LILRB2 in the presence of a candidate agent, wherein a reduction in the binding of TIM-3 and LILRB2 in the presence of the candidate agent by at least about 10% compared to binding of TIM-3 and LILRB2 in the absence of the candidate agent indicates that the agent inhibits the interaction of TIM-3 and LILRB2.

86-88. (canceled)

89: An antibody which specifically binds an epitope of TIM-3, wherein the epitope comprises the C′C″ and/or DE loop of TIM-3.

90-91. (canceled)

92: An antibody which specifically binds an epitope of TIM-3, wherein the epitope comprises the C′C″ loop of TIM-3.

93-95. (canceled)

96: An antibody which specifically binds an epitope of TIM-3, wherein the epitope comprises the DE loop of TIM-3.

97-114. (canceled)

115: A pharmaceutical composition comprising the antibody of claim 89 and a pharmaceutically acceptable carrier.
